SEQUENCE LISTING (5 sequences)

SEQ ID NO: 1 - DNA, 22 bp, Artificial Sequence
  Synthetic construct
ggacatatgatcaggagccgag

SEQ ID NO: 2 - DNA, 24 bp, Artificial Sequence
  Synthetic construct
aagtggtcatgtactagtcaagcg

SEQ ID NO: 3 - DNA, 24 bp, Artificial Sequence
  Synthetic construct
ggacatatgatcgtctcgatagtg

SEQ ID NO: 4 - DNA, 25 bp, Artificial Sequence
  Synthetic construct
caagtggtcatgtactagtcaatcc

SEQ ID NO: 5 - DNA, 84 bp, Artificial Sequence
  Synthetic construct
gggaagagaaggacauaugaucaggagccgagaaccgguugguggguaauccuguuagcgcuugacuaguacaugaccacuuga

(19) United States
(12) Patent Application Publication
(10) Pub. No.: US 2022/0370636 A1
(43) Pub. Date: Nov. 24, 2022

(54) NICOTINAMIDE ADENINE DINUCLEOTIDE ACTIVATOR AND USE THEREOF

(71) Applicant: University of South Florida, Tampa, FL (US)

(72) Inventors: Srinivas M. Tipparaju, Tampa, FL (US); Vijaykumar Sutariya, Tampa, FL (US)

(21) Appl. No.: 17/737,561

(22) Filed: May 5, 2022

Related U.S. Application Data
(60) Provisional application No. 63/184,457, filed on May 5, 2021.

(51) Int. Cl. A61K 47/69; A61K 9/51; A61K 9/00

(52) U.S. Cl. CPC A61K 47/6937; A61K 9/5153; A61K 9/5192; A61K 9/0019; B82Y 5/00

(57) ABSTRACT

Disclosed are methods for treating muscular diseases that involve the use of a nicotinamide adenine dinucleotide (NAD) activator.

### CROSS-REFERENCE TO RELATED APPLICATIONS

This application claims the benefit of U.S. Provisional Application No. 63/184,457, filed May 5, 2021, which is expressly incorporated herein by reference in its entirety.

### INCORPORATION-BY-REFERENCE OF MATERIAL SUBMITTED ELECTRONICALLY

Incorporated by reference in its entirety herein is a computer readable sequence listing submitted concurrently herewith and identified as follows: One 1,253 bytes ASCII (Text) file named “11001_060US1_Sequence_Listing,” created on May 4, 2022.

### FIELD

The present disclosure relates to nicotinamide adenine dinucleotide (NAD) activator and uses thereof for treating inflammatory diseases, muscle injury, arthritis, and joint pain.

### BACKGROUND

Skeletal muscle injury is a leading cause for decline in quality of life with increased mortality and morbidity associated with mechanical injury or age-related decline in muscle strength. Injury of muscle leads to either loss of muscle caused due to volumetric loss of muscle or due to muscular atrophy due to disuse. Over time, these factors lead to a decreased ability of muscle to fully recover and provide function. While anti-inflammatory (NSAIDs) agents (e.g., Ibuprofen, miloxicam), and steroids (e.g., dexamethasone) provide symptomatic relief and beneficial management of risks the issue of muscle repair and healing remain un-addressed in the field of skeletal muscle. Reducing inflammation alone cannot be the answer, as established treatments involving non-steroidal anti-inflammatory drugs (NSAIDs) are insufficient for full recovery of the muscle injury and is only provided for symptomatic relief. Therefore, what are needed are new molecules that directly address the need for resolving the inflammation and provide beneficial return of muscle to its pre-injury status and accelerate the healing process in a fundamentally new way, providing paradigm shifting ability by reimagining the skeletal muscle recovery process post-injury. What are also needed are new compositions and methods for treating inflammatory diseases, muscle injury, arthritis, and joint pain. The compositions and methods disclosed herein address these and other needs.

### SUMMARY

In accordance with the purposes of the disclosed materials and methods, as embodied and broadly described herein, the disclosed subject matter, in one aspect, relates to compounds, compositions and methods of making and using compounds and compositions. In specific aspects, the disclosed subject matter relates to methods for treating muscle injury, arthritis, and/or joint pain. In specific aspects, the disclosed subject matter relates to methods for treating a muscular disease. In specific aspects, the disclosed subject matter relates to methods for treating inflammatory diseases.

The details of one or more embodiments of the invention are set forth in the accompanying drawings and the description below. Other features, objects, and advantages of the invention will be apparent from the description and drawings, and from the claims.

Disclosed herein is a method of treating a muscular disease in a subject in need thereof, comprising administering to the subject a therapeutically effective amount of a nicotinamide adenine dinucleotide (NAD) activator or a pharmaceutically acceptable salt thereof. The disclosed small molecules help fundamentally change the cellular metabolism via providing NAD generation which helps with cellular function and tissue repair.

In some examples, the muscular disease is a muscle injury. In some examples, the muscle injury is a muscle strain injury. In some examples, the muscle injury is muscle atrophy. In some examples, the muscular disease is a diabetes-associated skeletal muscular disorder.

The NAD activator can improve muscle contractility, improve the regeneration of intramuscular nerves, and/or reduce a level of an inflammatory cytokine (including, e.g., IL-1β, IL-6, or TNF-α).

In some examples, the NAD activator is selected from the group consisting of 3,6-Dibromo-a-[(phenylamino)methyl]-9H-carbazole-9-ethanol (P7C3), P7C3 A20, P7C3 S243, and pharmaceutically acceptable salts thereof. In some examples, the NAD activator is P7C3 or a pharmaceutically acceptable salt thereof. P7C3 is an anti-inflammatory compound that attenuates inflammation via blocking Nfkb activation (master regulator of inflammation). P7C3 attenuates macrophage activation and due to decreased macrophage infiltration of injured skeletal muscle.

In some examples, the NAD activator is encapsulated within or associated with a nanoparticle. In some embodiments, the nanoparticle comprises poly(lactic-co-glycolic acid) (PLGA) or polyethylene oxide (PEG). In some embodiments, the nanoparticle comprises PLGA. In some embodiments, the nanoparticle comprises PEG.

Also disclosed herein is a method of preventing a muscular disease in a subject in need thereof, comprising administering to the subject a therapeutically effective amount of a nicotinamide adenine dinucleotide (NAD) activator or a pharmaceutically acceptable salt thereof. In some examples, the muscular disease is a muscle injury. In some examples, the muscle injury is a muscle strain injury. In some examples, the muscle injury is muscle atrophy. In some examples, the muscular disease is a diabetes-associated skeletal muscular disorder.

Also disclosed herein is a method of treating an inflammatory disease, comprising administering to a subject in need thereof a therapeutically effective amount of a nicotinamide adenine dinucleotide (NAD) activator.

Also disclosed herein is a nanoparticle comprising a polymer, a nicotinamide adenine dinucleotide (NAD) activator or a pharmaceutically acceptable salt thereof, and a muscle cell-targeting agent.

In some examples, the polymer comprises poly(lactic-co-glycolic acid) (PLGA) and/or polyethylene oxide (PEG). In some examples, the NAD activator is encapsulated within or associated with the nanoparticle. In some examples, the NAD activator is selected from the group consisting of 3,6-Dibromo-a-[(phenylamino)methyl]-9H-carbazole-9-ethanol (P7C3), P7C3 A20, P7C3 S243, and a pharmaceutically acceptable salt thereof. In some examples, the muscle cell-targeting agent is an A01B aptamer.

### BRIEF DESCRIPTION OF FIGURES

The accompanying figures, which are incorporated in and constitute a part of this specification, illustrate several aspects described below.

FIGS. 1A-1C show that NAD activation (via P7C3) leads to attenuation of BaCl₂ induced injury in TA muscle. FIG. 1A shows H&E staining; FIG. 1B shows % fibers, and FIG. 1C shows fusion index fold change. *p<0.05. FIG. 1A shows that C57BL6 mice tibialis anterior muscle was injected with BaCl₂ to induce inflammation. Saline or 50 μl of 1% BaCl₂ was injected in belly region of the muscle to induce inflammation. Muscle damage was assessed by H&E staining at day 7. The percentage of fibers recovered in higher in P7C3 treated group compared with saline treated group (FIG. 1B). The centrally placed nuclei were higher in P7C3 treated group with 3 or higher number of nuclei (FIG. 1C). These results demonstrate that P7C3 treatment attenuates inflammatory response in BaCl₂ model of muscle inflammation and allows for increased muscle recovery. * P<0.05, n=3 in each group.

FIGS. 2A and 2B show macrophage: LPS induced inflammation and recovery via P7 markers such as NF-κb, IL-1β, IL-6. FIG. 2A shows expression of the inflammatory, NF-κB and TNF-α, and anti-inflammatory, IL-10, Genes in LPS-P7C3-treated RAW264.7 cells in vitro. RAW264.7 cells was treated with LPS (1 μg/ml) and P7C3 (1 μM) for 24 hours. The isolated mRNA was subjected qRT-PCR using iScript cDNA Synthesis Kit (Bio-Rad, Cat #: 1706691) and iTaq Universal SYBR Green Supermix (Bio-Rad, Cat #: 1725121) in iCycler iQ Real Time PCR Detection System (Bio-Rad). The value of 2\hat{ }-ΔΔCt was calculated and expressed as a Mean±SEM (n=6). The statistical analysis was performed using two-way ANOVA with Tukey's multiple comparisons test in GraphPad Prism 8.4.3, and p≤0.05 was considered statistically significant. Abbreviation: ns, not significant; *, 0.05. FIG. 2B shows expression of the inflammatory, IL-1β, and the inflammatory and anti-inflammatory mytokine, IL-6, in LPS-P7C3-treated RAW264.7 cells in vitro. RAW264.7 cells was treated with LPS (1 μg/ml) and P7C3 (1 μM) for 24 hours. The isolated mRNA was subjected qRT-PCR using iScript cDNA Synthesis Kit (Bio-Rad, Cat #: 1706691) and iTaq Universal SYBR Green Supermix (Bio-Rad, Cat #: 1725121) in iCycler iQ Real Time PCR Detection System (Bio-Rad). The value of 2\hat{ }-ΔΔCt was calculated and expressed as a Mean±SEM (n=6). The statistical analysis was performed using two-way ANOVA with Tukey's multiple comparisons test in GraphPad Prism 8.4.3, and p≤0.05 was considered statistically significant. Abbreviation: ns, not significant; *, 0.05.

FIGS. 3A-3E show morphology and number of activated cells in LPS-induced RAW264.7 macrophage in vitro. RAW264.7 cells were cultured in complete medium (FIG. 3A) or contained with P7C3 (FIG. 3B, 1 μM), LPS (FIG. 3C, 1 μg/ml), and LPS+P7C3 (FIG. 3D) for 24. The photo was taken under inverted microscopy (Rebel, ECHO) at 20× mag. The elongated and pseudopod shape of cells were considered as activated (yellow star), but the small and round shape of cells was considered as inactivated (yellow arrow). FIG. 3E shows that the relative value was expressed as a Mean±SEM (n=7). The statistical analysis was performed using two-way ANOVA with Tukey's multiple comparisons test in GraphPad Prism 8.4.3, and p≤0.05 was considered statistically significant. Abbreviation: ns, not significant; *, 0.05; scale bar, 200 μm.

FIGS. 4A and 4B show MTT assay and morphology assay. FIG. 4A shows cell viability of LPS-induced RAW264.7 Cells in vitro. RAW264.7 cells was treated with LPS (1 μg/ml) and P7C3 (1 μM) for 24 hours. The assay of cell viability was performed as the instruction of the MTT cell proliferation assay kit (BioVision, cat #K299-1000). The value was expressed as a Mean±SEM in triplicate experiments (n=14). The statistical analysis was performed using RM one-way ANOVA with Tukey's multiple comparisons test in GraphPad Prism 8.4.3, and p≤0.05 was considered statistically significant. Abbreviation: ns, not significant. FIG. 4B shows amount of RNA in RAW264.7 cells treated with LPS and/or P7C3 for 24, 48, or 72 hours in vitro. RAW264.7 cells were cultured in 6-well culture plate overnight, and then treated with LPS (1 μg/ml) and/or P7C3 (1 μM) for 24, 48, or 72 hours. RNA was isolated using RNeasy Kit (Promega), and the amount of RNA was quantified using Nano-drop 1000. The amount of RNA was represented as Mean±SEM (n=6). The statistical analysis was performed using two-way ANOVA with Tukey's multiple comparisons test in GraphPad Prism 8.4.3, and p≤0.05 was considered statistically significant. Abbreviation: ns, not significant.

FIGS. 5A-5C show that C2C12 cells depict improved recovery for wound, and LPS induced inflammation. FIG. 5A shows that C2C12 cells depict improved recovery for wound (scratch) and LPS induced inflammation. In vitro model (C2C12 skeletal muscle myoblast) of scratch assay (wound) was created by using pipette tip and recovery of cells was monitored for 16 hours after the scratch. The percent wound closure was measured by the area indicated (FIG. 5B). Wound closure was significantly higher in P7C3 (1 μM) treated group and in the nano-formulated P7C3 (100 nm) treated cell groups. Note the significantly lower amount of P7C3 was used in the nano-P7C3 group showing that PLGA based Nano formulation of P7C3 is effective at a 10 times lower concentration. FIG. 5C shows that C2C12 cells were transformed to skeletal myotubes by using 4% horse serum for 72 hours. LPS treatment caused inflammation in vitro and was treatment with P7C3 (1 μM) 24 hours. FIG. 5C shows that LPS alone caused increased LDH release demonstration that LPS causes cell damage by inflammation, however +P7C3 treatment attenuated the inflammation in vitro. These data clearly demonstrate the anti-inflammatory effects of P7C3 in vitro. * P<0.05, n=3 in each group.

FIG. 6 shows nuclear translocation of pNFκB (ser 536) in LPS-activated RAW264.7 macrophage in vitro. RAW264.7 cells was treated with LPS (1 μg/ml) and P7C3 (1 μM) for 24 hours. The expression of NFkB was analyzed using western blotting. The value was calculated and expressed as a Mean±SEM (n=6). The statistical analysis was performed using Multiple t-test in GraphPad Prism 8.4.3, and p≤0.05 was considered statistically significant.

FIGS. 7A-7B: (FIG. 7A) Graphical representation scheme of A01B aptamer conjugation on P7C3 loaded PLGA-COOH NPs, and (FIG. 7B) animation of injured muscle tissue and efficient distribution of targeted NPs.

FIGS. 8A-8F show particle size and physical characters of nanoformulation: (FIG. 8A) Z-average and poly dispersity index (PDI) of P7C3 loaded PLGA-COOH NPs based on the dynamic light scattering technique using Nano ZS90, (FIG. 8B) Zeta Potential of P7C3 loaded PLGA-COOH NPs, (FIG. 8C) Z-average and PDI of Aptamer conjugated P7C3 loaded PLGA-COOH NPs based on the dynamic light scattering technique using Nano ZS90, (FIG. 8D) Zeta Potential of aptamer conjugated P7C3 loaded PLGA-COOH NPs. (FIG. 8E) TEM image of PLGA-COOH NPs, and (FIG. 8F) TEM images of aptamer conjugated PLGA-COOH NPs (scale bar 0.5 μm).

FIG. 9 shows cumulative drug release profile of P7C3 from drug solution and NPs in phosphate buffer saline at pH 7.4 at 37° C. for 7 days (mean±SD, n=3).

FIGS. 10A-10E show detection and measurement of A01B-aptamer in A01B aptamer-conjugated nanoparticles (NPs) entrapped P7C3 by RT-qPCR. The 0.1 μg of A01B aptamer (A01B) and Scrambled A01B aptamer (ScrA01B) that is conjugated or not with nanoparticles (NPs) with or without P7C3 (P7) were reverse-transcribed using cDNA synthesis kit (Bio-Rad), and 0.6 ng of cDNA equivalent was used for qPCR. Red (A01B-P7NPs), blue (A01B), pink (ScrA01B-P7NPs), green (ScrA01B-NPs), and black (others) color coding represent the different groups are used for the lines in FIGS. 10A and 10C. Amplification plot (FIG. 10A), CT value (FIG. 10B), Melting curve, (FIG. 10C) Table with groups color labeled for the A01B or Scrambled Aptamer group. (FIG. 10D) (i) A01B and (ii) scrambled A01B, Agarose gel electrophoresis (FIG. 10E). Abbreviation: M, 50 bp ladder (DNA molecular weight marker XIII, Roche Diagnostics); 1, P7NPs; 2, A01B-P7NPs; 3, ScrA01B-P7NPs; 4, ScrA01B-NPs; 5, NPs; 6, A01B.

FIGS. 11A-11D show quantification of A01B aptamer using RT-qPCR and standard curve: cDNA equivalent of 0.6, 0.15, 0.0375, and 0.0094 ng of A01B aptamer concentration was used to determine the standard curve with RT-qPCR analysis. Black (0.6 ng), blue (0.15 ng), yellow (0.0375), pink (0.0094 ng), and red (A01B-P7NPs) color coding represent the different groups are used for the lines in FIG. 11A and FIG. 11C. Amplification plot (FIG. 11A), Standard Curve (FIG. 11B), Melting peak (FIG. 11C), Amount of A01B aptamer in A01B-P7NPs in sample solution (FIG. 11D).

FIGS. 12A-12C show MTT assay for testing cell viability in vitro. Cell viability plots of C2C12 cells treated with different concentrations, i.e., 0, 10, 500, and 1000 nM (FIG. 12A) P7C3 drug solution, (FIG. 12B) P7C3 PLGA-COOH NPs, and (FIG. 12C) A01B aptamer targeted P7C3 PLGA-COOH at 24 h. The data are represented as mean±SEM, n=8/treatment group; *p<0.05).

FIGS. 13A-13B show assessment of cellular uptake of drug using fluorescence microscopy: (FIG. 13A) A01B (Targeted) and Scrambled (Non-targeted) aptamers were conjugated to PLGA-COOH NPs loaded with NBD-cholesterol dye (green). NP uptake was assessed by confocal microscopy imaging at 15 minutes time intervals for up to 60 minutes. DAPI (blue) nuclear and CellMask (red) dyes were used for nuclear and membrane staining, respectively, (FIG. 13B) Relative mean fluorescence intensity (MFI) was platted with change of time using ImageJ software for cellular uptake of NBD-cholesterol dye in C2C12 cells (*p<0.05).

FIGS. 14A-14F show the effect of regular P7C3 on the wound closure of C2C12 cells. (FIG. 14A) Images represent the wound closure of C2C12 cells at the 16 h time point after the scratch and treated with increasing concentrations of P7C3 (vehicle control (DMSO), 100 and 500 nM, and 1 and 2 μm). The region in the middle filled with white indicates the area measured using ImageJ. (FIG. 14B) The percentage wound closure for each concentration of P7C3 after 16 h. Data shown is mean±SEM, * p<0.05, n=4. (FIG. 14C) Images represent the wound closure of C2C12 cells at the 16 hours, treatments with NPs P7C3 (blank NPs, P7C3 equivalent to 10 nM, 100 nM, 500 nM, 1 μm). The region in the middle filled with white indicates the area measured using ImageJ. (FIG. 14D) The percentage wound closure for each concentration of P7C3 after 16 hours. Data shown is mean±SEM, * p<0.05, n=4. (FIG. 14E) Images that represent the wound closure of C2C12 cells at the 16 h time point after it was scratched and treated with 100 nM of A10B-P7C3 NPs and 100 nM scrambled P7C3 NPs. The region in the middle filled with white indicates the area measured using ImageJ. (FIG. 14F) The percentage wound closure for each concentration of P7C3 after 16 h. Data shown is mean±SEM, n=3.

FIGS. 15A-15B show the effect of regular P7C3 and nanoparticle P7C3 on the luciferase activity of inflammation-induced C2C12/NF-κB reporter cells. (FIG. 15A) The luminescence per protein unit (RLU*mL/mg) was measured for increasing concentration of regular P7C3 (vehicle control [DMSO+TNF-α], 100 nM, 500 nM, 1 μM) 24 hours after inflammation was induced by 10 ng/mL of TNF-α. (FIG. 15B) The luminescence per protein unit (RLU*mL/mg) was measured for increasing concentration of nanoparticle P7C3 (vehicle control [blank nanoparticle+TNF-α], 100 nM, 500 nM, 1 μM) 24 hours after inflammation was induced by 10 ng/mL of TNF-α. Data shown is mean±SEM. * p<0.05, n=8.

FIGS. 16A-16G show that P7C3 reverses hyperinsulinemia and hyperglycaemia of the diabetic mice. The 16-week-old type 2 diabetic (db/db) mice were administered daily with intraperitoneal dose of P7C3 (10 mg/kg/day) or vehicle for 4 weeks. (FIG. 16A) Weekly 6 h fasting blood glucose levels of the db/db mice treated with P7C3 or vehicle (n=11 each), and the C57Bl/6J WT (n=6) naïve control mice. (FIG. 16B) Insulin tolerance test (ITT) of 6 h fasted db/db mice injected with 1 U/kg body weight human insulin intraperitoneally (n=4-5 per group). (FIG. 16C) Glucose tolerance test (GTT) of overnight fasted db/db mice injected with 2 g/kg body weight D-(+)-glucose intraperitoneally and measured with GOD-POD method (n=5 mice per group). (FIG. 16D) The area under the curve of GTT was obtained using the trapezium method and expressed in percentage. (FIG. 16E) The circulating insulin levels in blood serum and (FIG. 16F) homeostatic model of insulin resistance (HOMA IR) and (FIG. 16G) homoeostatic model of pancreatic β-cells (HOMA B) expressed in percentage. Data are expressed as mean±SEM; *P<0.05; P7C3 vs. vehicle-treated db/db mice. The age-matched WT mice were used as naïve control to determine the baseline weekly fasting blood glucose levels.

FIGS. 17A-17F show that P7C3 increases the pancreatic β cells number and function of the db/db mice. (FIG. 17A) Immunohistochemical staining of insulin secreting pancreatic β cells (green). (FIG. 17B) The insulin secreting pancreatic β cells area per islet of Langerhans expressed as percentage. (FIG. 17C) Gomori aldehyde fuchsin staining of pancreas. (FIG. 17D) The number of pancreatic β cells per section, and (FIG. 17E) number of islet of Langerhans per section and (FIG. 17F) number of pancreatic β cells per islet of Langerhans. Data are expressed as mean±SEM; *P<0.05; P7C3 vs. vehicle-treated db/db mice.

FIGS. 18A-18G show that P7C3 enhances physical performance of db/db mice. (FIG. 18A) Fore-limb grip strength and (FIG. 18B) hind-limb grip strength measured and expressed as KGF/kg body weight. (FIG. 18C) The absolute force frequency and (FIG. 18D) normalized force frequency of the extensor digitorum longus muscles. (FIG. 18E) The area un-der the curve of the absolute muscle force frequency and (FIG. 18F) the normalized muscle force frequency. (FIG. 18G) The voluntary running wheel performance of the mice expressed in metres. Data are expressed as mean±SEM; *P<0.05; P7C3 vs. vehicle-treated db/db mice. The age-matched WT mice were used as naïve control to determine the baseline muscle force frequencies of the extensor digitorum longus muscle.

FIGS. 19A-19I show that P7C3 ameliorates the diabetic skeletal muscle phenotype of the db/db mice. (FIG. 19A) Haematoxylin and eosin stained ×20 magnification images of the tibialis anterior muscles of the diabetic mice treated with vehicle or P7C3 and the wild-type naïve control mice. (FIGS. 19B, 19C) analysis of 250 random myofibres cross-sectional area of the haematoxylin and eosin-stained images per section and 3 such section was counted per mice, where n=3 mice per group displaying a shift towards medium-sized myofibres in the db-P7C3 treated mice compared to db-Veh treated mice. The age-matched WT naïve control mice myofibres display an increased larger sized myofibres. (FIG. 19D) Transmission electron microscopy images of the extensor digitorum longus muscle of the WT naïve control, db-Veh and db-P7C3 treated mice. The intermyofibrillar mitochondria (IM), and the Z-lines (arrows) are distinguished in the TEM images taken at ×30,000 magnification. (FIG. 19E) Analysis of the extensor digitorum longus muscles myofibre diameter of TEM images at ×30,000 magnifications (n=8-9 myofibres per image, and 3 such images were analysed per group). Data are expressed as mean±SEM; *P<0.05. (FIG. 19F) Mitochondrial area measured from db-Veh or db-P7C3 expressed as μm². (FIG. 19G) Succinate dehydrogenase (SDH) staining of TA muscle, (FIG. 19H) quantification of SDH stained myofibres, and (FIG. 19I) relative mRNA expression of 16 S in wild-type (WT), db-Veh, and db-P7C3. Data are expressed as mean±SEM, P<0.05.

FIGS. 20A-20F show that P7C3 decreases MyHC1 expression levels and immunostained myofibres numbers in gastrocnemius muscle. Relative gene expression levels of myosin heavy chain fibre types: (FIG. 20A) MyHC1, (FIG. 20B) MyHC2a, (FIG. 20C) MyHC2b, and (FIG. 20D) MyHC2×. (FIG. 20E) Immunolabelling of tibialis anterior muscle cryosections of WT, db-Veh, and db-P7C3 treated mice. MyHC1 positive myofibres are immunostained in grey. (FIG. 20F) Quantification of MyHC1 depicting the fold change in grey myofibre immunostaining. Data are expressed as mean±SEM; *P<0.05. The age-matched WT mice was used as naïve control to determine the baseline expression levels of the myosin heavy chain fibre types in gastrocnemius muscle and immunostaining of MyHC1 in tibialis anterior muscle.

FIGS. 21A-21J show that P7C3 treatment increases fatty acid oxidation and decrease myofibre stress in db/db mice. Relative mRNA expression levels of (FIG. 21A) Fabp1, (FIG. 21B) CD36, (FIG. 21C) Pdk4, (FIG. 21D) Cpt1, (FIG. 21E) Fgf21, and (FIG. 21F) schematic depiction of differential gene responses in db-P7C3 and db-Veh showing increased mitochondrial fatty acid oxidation and decreased fatty acid uptake and oxidative stress with P7C3 treatment in diabetic mice (FIG. 21G) RNA-seq analysis of skeletal muscle from db-Veh and db-P7C3 (FIG. 21H) volcano plot showing differentially expressed genes (DEG) in db_P7C3-db_Veh. The up-regulated genes (light grey), and the down-regulated genes (dark grey) with a fold change <0.6, and with P<0.05 (FIG. 21I) Venn diagram showing comparison of the differentially up-regulated genes in db_P7C3-db_Veh and down-regulated genes in db_Veh-WT_Veh, and provides the P7C3 treatment responsive genes with 772 genes up-regulated and 1213 genes down-regulated, respectively (FIG. 21J) the top 17 up-regulated and 16 down-regulated pathways in the biological processes (BPs), chemical component (CC), Kyoto encyclopaedia of genes and genomes (KEGG) and molecular function (MF) pathways. Data expressed is mean±SEM, *P<0.05. The age-matched WT mice was used as naïve control to determine the baseline expression levels of the key genes involved in fatty acid uptake, oxidation, and oxidative stress in gastrocnemius muscle.

FIG. 22 shows that cholesterol and lipid mediators are differentially altered in P7C3 treated db/db mice. Levels of serum IP-10 (C—X—C motif chemokine ligand 10) measured by Luminex-MagPix magnetic bead immunoassay. Data are expressed as mean±SEM; *P<0.05. The age-matched WT mice were used as naïve control to determine the baseline lipid mediators levels in gastrocnemius muscle.

FIGS. 23A-23C show that P7C3 treatment improves the Nampt enzymatic activity. (FIG. 23A) Enzymatic activity of recombinant Nampt was measured every 5 min for 1 h. Negative control (DMSO), positive control (Nampt enzyme), Nampt inhibitor (FK866), and Nampt activator (P7C3) were all utilized to assess the enzy-matic activity. (FIG. 23B) The gastrocnemius muscle Nampt enzymatic activity was measured every 5 min for a total duration of 40 min. Data are expressed as mean±SEM; *P<0.05 of the absorbance measured at 20 min during the linear range. (FIG. 23C) Serum pyruvate activity of db-Veh and db-P7C3 treated mice. Data expressed as mean±SEM with P<0.05.

FIGS. 24A-24D show gene network interaction using Cytoscape v3.8.2 for the up-regulated genes as a STRING network in (FIG. 24A) MCODE (molecular complex detection) used to identify dense clusters with enrichment scores in FIGS. 24B-24D top three clusters. Cluster 1 has enrichment score=17.579, Cluster 2 has enrichment score=8.500, and Cluster 3 has enrichment score=8.286.

FIGS. 25A-25D show gene network interaction using Cytoscape v3.8.2 for the down-regulated genes as a STRING network in (FIG. 25A) MCODE (molecular complex detection) was used to identify dense clusters with enrichment scores in FIGS. 25B-25D top three clusters. Cluster 1 has enrichment score=38.789, Cluster 2 has enrichment score=29.760, and Cluster 3 has enrichment score=16.833.

FIGS. 26A-26E show physical performance of wild-type and db-Veh mice, and Tnfα expression. (FIG. 26A) Forelimb grip strength and (FIG. 26B) Hindlimb grip strength measured and expressed as KGF/kg body weight (FIG. 26C) The area under the curve (AUC) of the absolute muscle force frequency, and (FIG. 26D) the normalized muscle force frequency of the extensor digitorum longus muscles (FIG. 26E). The relative mRNA expression levels of Tnfα in db-Veh and db-P7C3 gastrocnemius muscle. Data are expressed as mean±SEM; *P<0.05.

FIGS. 27A-27F show that P7C3 reverses hyperglycaemia in a Nampt-dependent manner. (FIG. 27A) Genotype of Nampt littermate wildtype (WT) with a single 300 bp nucleotide band and Nampt heterozygous mice (Nampt+/−) with a double band, i.e., 300 bp wildtype and the 500 bp heterozygous nucleotide bands. (FIG. 27B) Forelimb grip strength of the Nampt+/+ WT and Nampt+/− mice expressed in KGF/kg. (FIG. 27C) Glucose tolerance test of the Nampt+/+ wild type and (FIG. 27D) Nampt+/− heterozygous mice. Percentage of area under curve (AUC) for IPGTT obtained by using trapezium method for both the (FIG. 27E) Wildtype and (FIG. 27F) Nampt+/− heterozygous mice. Data are expressed as mean±SEM; *P<0.05 of P7C3 vs. Vehicle treated in wild type and Nampt+/− heterozygous mice.

FIG. 28A shows construction of Aptamer (A01B) based targeted drug delivery to skeletal muscle, ECR epitopes recognize the sk. muscle, NH2 linker conjugates with PLGA-drug, hairpin loop structure allows for stability, Cy3 is used for tracking. FIG. 28B shows that synthesis of PLGA-b-PEG-COOH nanoparticles, and conjugation of aptamer to nanoparticles. Drug (P7C3 or glucocorticosteroids) was encapsulated within PLGA-b-PEG-COOH nanoparticles using the nanoprecipitation method. The PLGA-PEG nanoparticles/drug was covalently conjugated to amine-terminated A01B Aptamer in the presence of EDC. The sequence in the FIG. 28A is GGGAAGAGAAGGACAUAUGAUCAGGAGCCGAGAACCGGUUGGUGGGUAAUCCUG UUAGCGCUUGACUAGUACAUGACCACUUGA (SEQ ID NO: 5).

FIGS. 29A-29C show that P7C3 treatment improves enzymatic Nampt activity. (FIG. 29A) Vehicle (db/db) hearts demonstrated a decrease in Nampt activity compared with wildtype control (WT), while 4-week treatment with P7C3 (10 mg/kg body weight/day, i.p.) in db/db mice resulted in significantly increased Nampt activity. (FIGS. 29B-29C) Enzymatic activity of recombinant Nampt was measured every 5 minutes for 1 hour. Negative control (DMSO), positive control (Nampt enzyme), Nampt activator (P7C3) were all utilized to assess enzymatic activity. Data was reported as mean±SEM and * indicated a p≤0.05.

FIGS. 30A-30H show computational modeling of P7C3 docking with Nampt active site. (FIG. 30A) Chemical structure of P7C3 with 2-D representation of the molecule. (FIG. 30B) Table provides the relative binding free energy of P7C3 when docked with the Nampt protein (from PDB code 4WQ6) and lists the projected distances (measured in angstroms) between selected regions of the P7C3 compound and related regions of the Nampt binding site. (FIGS. 30C-30F) Image depicts (P7C3 ligand in black), wherein (FIGS. 30C-30D) the top view, (FIGS. 30E-30F) side view, of the Nampt binding site (from PDB code 4WQ6). Green shading illustrates the van der Waals surface of the Nampt binding site. The protein ribbon structure shown in the image is provided to emphasize the Nampt binding site (“aka tunnel”) when two Nampt monomers dimerize to form the Nampt dimeric interface. (FIG. 30E) Image depicts the projected interactions by GLIDE SP docked P7C3 within the Nampt binding site (from PDB code 4WQ6). As it appears the GLIDE SP docked pose of P7C3 demonstrates no specific interactions between P7C3 and the Nampt binding site. Although a single interaction does exist between P7C3 and a water molecule (depicted as a purple dot) as indicated by the hydrogen bond represented as the black dashed line. (FIG. 30F) Image shows the interaction between the Nampt monomers to form the active site cavity. (FIG. 30G) Image displays the entire Nampt dimeric protein where monomer A is displayed in green and monomer B is displayed in blue and the yellow shading illustrates the van der Waals surface of the Nampt binding site (“aka tunnel”) of each interface. (FIG. 30H) Image emphasizes the two active sites (i.e., catalytic sites) contained within the Nampt dimeric protein.

FIGS. 31A-31C show that P7C3 treatment improves blood glucose levels of the diabetic mice. (FIG. 31A) Fasting blood glucose levels of the db/db mice treated with P7C3 (10 mg/kg body weight/day, i.p.) (db/db P7C3) or with an equivalent volume of vehicle (db/db Veh) (n=5 mice/group). Data are expressed as mean±SEM, and * indicated a p≤0.05. (FIG. 31B) The intraperitoneal glucose tolerance test (GTT) of the overnight fasted db/db Veh and db/db P7C3 mice (n=5-6 mice/group). (FIG. 31C) The area under the curve of GTT obtained by using the trapezium method. There was a significant difference between the treatment groups variances * indicated a p=0.0021.

FIGS. 32A-32D show that administration of P7C3 preserves ECG in db/db mice. ECG recordings were acquired in db/db mice after a 4-week treatment period with P7C3 (10 mg/kg body weight/day, i.p.) or vehicle (10 ml/kg body weight/day i.p.) control. (FIG. 32A) QTc interval (FIG. 32B) JT interval, (FIG. 32C) ST elevation (mV), (FIG. 32D) QTc and Nampt Activity correlation. Data are expressed as means±SEM*p<0.05; P7C3 vs. vehicle treated db/db mice. Correlation is Spearman.

FIGS. 33A-33E show that P7C3 improves contractile function in db/db mice. Echocardiography acquired from db/db mice post 4-week treatment with P7C3 (10 mg/kg body weight/day, i.p.) or vehicle. (FIG. 33A) Representative M-mode short axis images from db/db mice treated with vehicle and P7C3. (FIG. 33B) Ejection fraction measurements from db/db vehicle and P7C3 (FIG. 33C) Fractional shortening measurements from db/db vehicle and P7C3. (FIG. 33D) Body weight measurements from db/db vehicle and P7C3. (FIG. 33E) Left ventricular heart weight measurements from db/db vehicle or P7C3. Data are expressed as means±SEM; *p<0.05 P7C3 vs. vehicle treated db/db mice.

FIGS. 34A-34F show that P7C3 treatment potentiates AKT phosphorylation and cardioprotective signaling via eNOS and autophagy upregulation with enhanced SiRT-1 activity in db/db mice. After 4-week treatment with P7C3 (10 mg/kg body weight/day, i.p.) or vehicle, db/db mice were injected intravenously (iv.) with 1 unit/kg Novolin R regular human insulin. After 5 minutes, hearts were collected and snap frozen in liquid N₂. (FIG. 34A) Immunoblots of whole heart lysates were analyzed by Western blotting for cardiac levels of phosphorylated AKT^{Ser473} (p-AKT), total AKT (t-AKT) and the ratio of p-AKT/t-AKT. (FIG. 34B) Cardiac levels of phosphorylated eNOS (p-eNOS), total eNOS (t-eNOS) and the ratio of p-eNOS/t-eNOS. (FIG. 34C) Western blotting of Beclin−1, GAPDH and the ratio of Beclin−1/GAPDH. Band intensities were quantified using image J software reported as mean±SEM, *p<0.05, ** for vehicle vs. P7C3. (FIG. 34D) Sirt-1 activity data are expressed as means±SEM ***p<0.001; P7C3 vs. vehicle treated db/db mice. (FIG. 34E) Nampt activity and Sirt-1 activity correlation. Correlation is Spearman. (FIG. 34F) P7C3 treated db/db mice demonstrated elevated cardiac NAD/NADH levels compared with db/db vehicle controls.

FIGS. 35A-35C show ischemia reperfusion injury in db/db hearts treated with P7C3. Ex vivo ischemia reperfusion injury was assessed via the Langendorff perfusion system. (FIG. 35A) Schematic of Ischemia reperfusion protocol utilized. (FIG. 35B) TTC staining of I/R hearts from db/db mice perfused with P7C3 (3 μM) or vehicle with representative section images. (FIG. 35C) Cardiac effluents were assessed for Troponin I taken at baseline, 30 minutes post Ischemia/Reperfusion, 60 minutes post, and 120 minutes. Effluent samples represent (n=4) means±SEM*p<0.05; P7C3 vs. vehicle treated db/db mice.

FIGS. 36A-36F show ischemia reperfusion injury in wildtype mice perfused with P7C3. Ex vivo ischemia reperfusion injury was assessed via the Langendorff perfusion system. (FIG. 36A) Schematic of Ischemia reperfusion protocol utilized. (FIG. 36B) TTC staining of I/R hearts from WT mice perfused with P7C3 (3 μM) or vehicle with representative section images. Heart samples presented are (n=4) means±SEM ****p<0.0001; P7C3 vs. vehicle treated WT mice. (FIG. 36C) WT hearts perfused with vehicle and P7C3 (3 μM) had Nampt activity levels determined post treatment. (FIG. 36D) WT hearts perfused with vehicle and P7C3 (3 μM) had cardiac NAD/NADH levels determined post treatment. (FIG. 36E) Troponin I measurements taken at baseline, 30 minutes post Ischemia/Reperfusion, 60 minutes post, and 120 minutes. (FIG. 36F) LDH measurements taken at baseline, 30 minutes post Ischemia/Reperfusion, 60 minutes post, and 120 minutes. Data was reported as mean±SEM and * p≤0.05, **p≤0.01, ***p≤0.001, ***p≤0.0001.

FIGS. 37A-37D show that ischemia reperfusion injury results in dysregulated pAKT. Ex vivo ischemia reperfusion injury was assessed via the Langendorff perfusion system and heart tissue was examined for pAKT. (FIG. 37A) Immunoblots of whole heart lysates were analyzed by Western blotting for cardiac levels of phosphorylated AKT ser473 (p-AKT), total AKT (t-AKT) and the ratio of p-AKT/t-AKT. Band intensities were quantified using image J software reported as mean±SEM, *p<0.05, for control and DMSO vs. P7C3 (3 μM). (FIG. 37B) Schematic of Ischemia-Ly294002 (15 μM) reperfusion and protocol utilized. (FIG. 37C) Immunoblots of whole heart lysates were analyzed by Western blotting for cardiac levels of phosphorylated AKT ser473 (p-AKT), total AKT (t-AKT) and the ratio of p-AKT/t-AKT. Band intensities were quantified using image J software reported as mean±SEM, ***p<0.001, for Ly294002+P7C3 vs. P7C3. (FIG. 37D) Schematic diagram showing the effects of P7C3 for cardioprotection.

FIGS. 38A-38D show ischemia reperfusion injury in wildtype mice post-ischemia perfused with P7C3. (FIG. 38A) Ex vivo ischemia reperfusion injury was assessed via the Langendorff perfusion system. Schematic of Ischemia reperfusion protocol utilized. (FIG. 38B) TTC staining of I/R hearts from WT mice perfused with P7C3 (3 μM) or vehicle with representative heart tissue sections. (FIG. 38C) In vivo permanent ligation model used to assess the infarct size and benefit via using P7C3. The C57Bl/6J wildtype (WT) mice were treated with a single bolus of P7C3 (10 mg/kg body weight/day, i.p.) or an equivalent dose of vehicle 30 minutes prior to ligating the left anterior descending (LAD) artery. Mice were euthanized at 24-hour time point and the heart samples were processed for the infarct size measurement using TTC staining (FIG. 38D). Heart samples presented are (n=3) means±SEM **p<0.01; P7C3 vs. vehicle treated WT mice.

### DETAILED DESCRIPTION

Terms used throughout this application are to be construed with ordinary and typical meaning to those of ordinary skill in the art. However, Applicant desires that the following terms be given the particular definition as defined below.

### Terminology

As used in the specification and claims, the singular form “a,” “an,” and “the” include plural references unless the context clearly dictates otherwise. For example, the term “a cell” includes a plurality of cells, including mixtures thereof.

The term “about” as used herein when referring to a measurable value such as an amount, a percentage, and the like, is meant to encompass variations of 20%, +10%, ±5%, or 10% from the measurable value.

“Activate”, “activating”, and “activation” mean to increase an activity, response, condition, or other biological parameter. This may also include, for example, a 10% increase in the activity, response, “or condition, as compared to the native or control level. Thus, the increase can be a 10, 20, 30, 40, 50, 60, 70, 80, 90, 100%, or any amount of reduction in between as compared to native or control levels.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

The term “agonist” or “activator” refers to a composition that binds to a receptor and activates the receptor to produce a biological response. The terms also encompass pharmaceutically acceptable, pharmacologically active derivatives of agonists specifically mentioned herein, including, but not limited to, salts, esters, amides, proagents, active metabolites, isomers, fragments, analogs, and the like. When the term “agonist” or “activator” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc. Accordingly, the term “NAD agonist” or “NAD activator” can include any one or more agents which upon administration to a subject, can activate NAD or the related pathways.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

The term “biocompatible” generally refers to a material and any metabolites or degradation products thereof that are generally non-toxic to the recipient and do not cause significant adverse effects to the subject.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

A “control” is an alternative subject or sample used in an experiment for comparison purposes. A control can be “positive” or “negative.”

“Decrease” can refer to any change that results in a lower level of gene expression, protein expression, amount of a symptom, disease, composition, condition, or activity. A substance is also understood to decrease the level of the gene, the protein, the composition, or the amount of the condition when the level of the gene, the protein, the composition, or the amount of the condition is less/lower relative to the output of the level of the gene, the protein, the composition, or the amount of the condition without the substance. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, Pa., 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, N.J.), polyethylene glycol (PEG), and PLURONICS™ (BASF; Florham Park, N.J.). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

“Pharmaceutically acceptable salts” as used herein refer to any salt that is pharmaceutically acceptable and has the desired pharmacological properties. Such salts are, for example, inorganic or organic base addition salts, or inorganic or organic acid addition salts. Each possibility represents a separate embodiment.

As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

“Increase” can refer to any change that results in a higher level of gene expression, protein expression, amount of a symptom, disease, composition, condition, or activity. A substance is also understood to increase the level of the gene, the protein, the composition, or the amount of the condition when the level of the gene, the protein, the composition, or the amount of the condition is more/higher relative to the output of the level of the gene, the protein, the composition, or the amount of the condition without the substance. Also, for example, an increase can be a change in the symptoms of a disorder such that the symptoms are less than previously observed. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

The term “subject” refers to a human in need of treatment for any purpose, and more preferably a human in need of treatment to treat an inflammatory disease. The term “subject” can also refer to non-human animals, such as non-human primates.

As used herein, the terms “treating” or “treatment” of a subject includes the administration of a drug to a subject with the purpose of curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, and improvement or remediation of damage.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g., a composition comprising an agent) refers to an amount that is effective to achieve a desired therapeutic result. In some examples, a desired therapeutic result is the control of an inflammatory disease or a symptom thereof. In some examples, a desired therapeutic result is the control of a muscular disease or a symptom thereof. In some examples, a desired therapeutic result is improvement of muscle contractility and function. In some examples, a desired therapeutic result is improvement of the regeneration of intramuscular nerves. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The term “nanoparticle” as used herein refers to a particle or structure which is biocompatible with and sufficiently resistant to chemical and/or physical destruction by the environment of such use so that a sufficient number of the nanoparticles remain substantially intact after delivery to the site of application or treatment and whose size is in the nanometer range. For the purposes of the present invention, a nanoparticle typically ranges from about 1 nm to about 1000 nm, preferably from about 50 nm to about 500 nm, more preferably from about 200 nm to about 300 nm.

The term “polymer” as used herein refers to a relatively high molecular weight organic compound, natural or synthetic, whose structure can be represented by a repeated small unit, the monomer. Synthetic polymers are typically formed by addition or condensation polymerization of monomers. The polymers used or produced in the present invention are biodegradable. The polymer is suitable for use in the body of a subject, i.e., is biologically inert and physiologically acceptable, non-toxic, and is biodegradable in the environment of use, i.e. can be resorbed by the body. Examples of synthetic polymers include, but are not limited to, poly(lactic-co-glycolic acid) (PLGA). The term “polymer” encompasses all forms of polymers including, but not limited to, natural polymers, synthetic polymers, homopolymers, heteropolymers or copolymers, addition polymers, etc.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Compounds and Methods

Inflammation and pain in skeletal muscle is a major problem. The methods and compositions disclosed herein can improve the intracellular content of nicotinamide adenine dinucleotide (NAD) to reduce inflammation and enhance recovery. The compositions and methods disclosed herein can have broad implication in the field of inflammation and pain fields.

Accordingly, disclosed herein is a method of treating and/or preventing a muscular disease (e.g., a muscle injury, arthritis, or joint pain) comprising administering a therapeutically effective amount of a nicotinamide adenine dinucleotide (NAD) activator or a pharmaceutically acceptable salt thereof to a subject in need thereof. In some embodiments, the disease is a diabetes-associated skeletal muscular disorder. In some embodiments, the muscular disease is a diabetes-associated cardiac disorder. As described herein, small molecules are identified that provide anti-inflammatory properties to decrease inflammatory processes and resolve the injury. In vitro and in vivo approaches disclosed herein demonstrate the utility and application of small molecules for inflammation resolution and aid in repair of skeletal muscle and related pain. The main mode of inflammation is transmitted via immunogenic cells (macrophage, Nfkb, and cytokines) and it is demonstrated herein that the small molecule (NAD activator) decreases the master regulators of inflammation.

Nicotinamide adenine dinucleotide (NAD) or NAD⁺ is a cofactor central to metabolism. Found in all living cells, NAD is a dinucleotide as it consists of two nucleotides joined through their phosphate groups. One nucleotide contains an adenine nucleobase and the other nicotinamide.

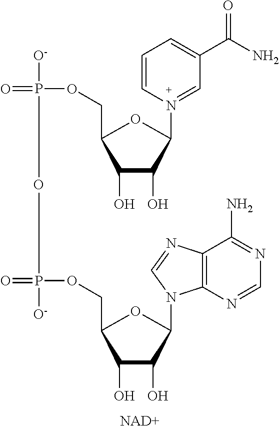

NAD plays a vital role in diverse cellular processes that govern human health and disease. NAD is involved in cellular processes including cell signaling, DNA repair, cell division, and epigenetics. Elevated tissue levels of NAD⁺ were linked to salutary effects including healthy aging. NAD synthesis requires the nicotinamide (NAM) salvage pathway that involves sequential actions of nicotinamide phosphoribosyltransferase (NAMPT) and NMN adenylyltransferases (NMNAT1-3). Accordingly, in some examples, the term “NAD” activator used herein refers to an agent that increases the level of NAD, for example, an activator of NAMPT or NMN. Activation of the described target protein is achieved when the activity value relative to a reference control is at least about 110%, optionally at least about 150%, optionally at least about at least about 200, at least about 300%, at least about 400%, at least about 500%, or at least about 1000-3000% or more. In some examples, the level of NAD may be enhanced by at least about 5% (e.g., at least about 10%, at least about 20%, at least about 30%, at least about 40%, at least about 50%, at least about 60%, at least about 70%, at least about 80%, at least about 90%, at least about 100%, at least about 200%, at least about 500%, at least about 1000%, or at least about at least about 3000%) as compared to a reference control. The term “reference control” refers to a level in detected in a subject in general or a study population (e.g., healthy control or a subject without the administration of the composition).

In some examples, the NAD activator is selected from the group consisting of 3,6-Dibromo-a-[(phenylamino)methyl]-9H-carbazole-9-ethanol (P7C3), P7C3 A20, P7C3 S243, and pharmaceutically acceptable salts thereof. In some examples, the NAD activator is P7C3 or a pharmaceutically acceptable salt thereof.

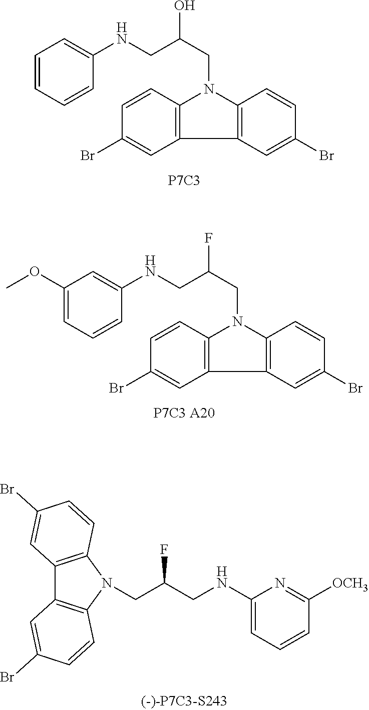

In some examples, the muscle injury is a muscle strain injury, such as the sports-related muscle injuries. In some embodiments, the muscle injury is a disorder associated with reduced contractile force, such as muscle atrophy, failure to regenerate intramuscular nerves, and fibrosis. In some examples, the muscle injury is muscle atrophy. The term “muscle atrophy” herein refers to a disease associated with a decline in skeletal muscle mass and function. Accordingly, the methods and compositions disclosed herein can mitigate/inhibit/prevent/treating the decline in skeletal muscle mass and/or function. In some embodiments, the muscular disease is a diabetes-associated skeletal muscular disorder. In some embodiments, the muscular disease is Diffuse idiopathic skeletal hyperostosis (DISH), Dupuytren's contracture, frozen shoulder, Charcot joint. In some embodiments, the muscular disease is a diabetes-associated cardiac disorder. In some embodiments, the methods and compositions disclosed herein are effective to treat or prevent a cardiovascular disease or disorder and/or can be cardioprotective. In some embodiments, disclosed herein are methods and compositions for treatment of cardiovascular disease or disorder in a subject in need. In some embodiments, the subject has diabetes. In some embodiments, the subject does not have diabetes. In some embodiments, the subject has ischemia reperfusion injury. Accordingly, the compositions and methods disclosed herein can reduce one or more the symptoms of the muscular disease, including, for examples, swollenness of the joints, reduced mobility of the joints, and/or reduced contractility of muscle.

Accordingly, disclosed herein are methods of treating and/or preventing a skeletal muscular disorder in a subject in need thereof, comprising administering to the subject a therapeutically effective amount of the compositions or compounds described herein.

In some aspects, disclosed herein are methods of treating and/or preventing a diabetes-associated skeletal muscular disorder in a subject in need thereof, comprising administering to the subject a therapeutically effective amount of the compositions or compounds described herein.

In some aspects, disclosed herein are methods of treating and/or preventing a diabetes-associated cardiac disorder in a subject in need thereof, comprising administering to the subject a therapeutically effective amount of the compositions or compounds described herein.

In some aspects, disclosed herein are methods of treating and/or preventing ischemia reperfusion injury in a subject in need thereof, comprising administering to the subject a therapeutically effective amount of the compositions or compounds described herein.

The compositions and methods disclosed herein are effective on treating and/or preventing one or more of decline in muscle mass and function, reduction in contractile force, and/or increased inflammation. Accordingly, the compositions and methods disclosed herein can improve post-strain injury recovery of muscle, the regeneration of intramuscular nerves, and muscle contractility, and/or reduce inflammation compared to a reference control. In some examples, the NAD activator improves muscle contractility. In some examples, the NAD activator reduces a level of an inflammatory cytokine (including, for example, IL-1β, IL-6, or TNF-α). In some examples, the compositions and methods disclosed herein can protect/prevent muscle and neuron from injury, The term “reference control” refers to a level in detected in a subject in general or a study population (e.g., healthy control or a subject without the administration of the composition).

Also, disclosed herein is a method of treating and/or preventing an inflammatory disease, comprising administering to a subject in need thereof a therapeutically effective amount of a nicotinamide adenine dinucleotide (NAD) activator.

In some examples, the NAD activator is encapsulated within or associated with a nanoparticle.

The nanoparticle used herein can be any nanoparticle useful for the delivery of nucleic acids and/or polypeptides. The term “nanoparticle” as used herein refers to a particle or structure which is biocompatible with and sufficiently resistant to chemical and/or physical destruction by the environment of such use so that a sufficient number of the nanoparticles remain substantially intact after delivery to the site of application or treatment and whose size is in the nanometer range. In some embodiments, the nanoparticle comprises a lipid-like nanoparticle. See, for example, WO/2016/187531A1, WO/2017/176974, WO/2019/027999, or Li, B et al. An Orthogonal array optimization of lipid-like nanoparticles for mRNA delivery in vivo. Nano Lett. 2015, 15, 8099-8107; which are incorporated herein by reference in their entireties. In some embodiments, the nanoparticle comprises poly (lactide-co-glycolide) (PLGA).

Nanoparticles disclosed herein include one, two, three or more biocompatible and/or biodegradable polymers. For example, a contemplated nanoparticle may include about 10 to about 99 weight percent of a one or more block co-polymers that include a biodegradable polymer and polyethylene glycol, and about 0 to about 50 weight percent of a biodegradable homopolymer. Polymers can include, for example, both biostable and biodegradable polymers, such as microcrystalline cellulose, hydroxypropyl cellulose, hydroxypropyl methylcellulose, polyalkylene oxides such as polyethylene oxide (PEG), polyanhydrides, poly(ester anhydrides), polyhydroxy acids such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof. In some embodiments, the nanoparticle comprises PLGA. In some embodiments, the nanoparticle comprises PEG.

In some embodiments, the nanoparticle has a diameter from about 1 nm to about 1000 nm. In some embodiments, the nanoparticle has a diameter less than, for example, about 1000 nm, about 950 nm, about 900 nm, about 850 nm, about 800 nm, about 750 nm, about 700 nm, about 650 nm, about 600 nm, about 550 nm, about 500 nm, about 450 nm, about 400 nm, about 350 nm, about 300 nm, about 290 nm, about 280 nm, about 270 nm, about 260 nm, about 255 nm, about 250 nm, about 240 nm, about 230 nm, about 220 nm, about 210 nm, about 200 nm, about 190 nm, about 180 nm, about 170 nm, about 160 nm, about 150 nm, about 140 nm, about 130 nm, about 120 nm, about 110 nm, about 100 nm, about 90 nm, about 80 nm, about 70 nm, about 60 nm, about 50 nm, about 40 nm, about 30 nm, about 20 nm, or about 10 nm. In some embodiments, the nanoparticle has a diameter, for example, from about 20 nm to about 1000 nm, from about 20 nm to about 800 nm, from about 20 nm to about 700 nm, from about 30 nm to about 600 nm, from about 30 nm to about 500 nm, from about 40 nm to about 400 nm, from about 40 nm to about 300 nm, from about 40 nm to about 250 nm, from about 50 nm to about 250 nm, from about 50 nm to about 200 nm, from about 50 nm to about 150 nm, from about 60 nm to about 150 nm, from about 70 nm to about 150 nm, from about 80 nm to about 150 nm, from about 90 nm to about 150 nm, from about 200 nm to about 300 nm, from about 220 nm to about 270 nm, or from about 230 nm to about 260 nm.

A nanoparticle has a surface charge that attracts ions having opposite charge to the nanoparticle surface. Such a double layer of ions travels with the nanoparticle. Zeta potential refers to the electrostatic potential at the electrical double layer. In some embodiments, the nanoparticle disclosed herein has a zeta potential ranging from about +10 mV to about +100 mV, about +5 mV to about +20 mV, about +7 mV to about +15 mV, about +7 mV to about +12 mV, more than about +5 mV, more than about +6 mV, more than about +7 mV, more than about +9 mV, more than about +10 mV, more than about +11 mV, more than about +12 mV, more than about 13 mV, more than about +14 mV, more than about +15 mV, more than about +16 mV, more than about +17 mV, more than about +18 mV, more than about +19 mV, more than about +20 mV, more than about +21 mV, more than about +22 mV, more than about +23 mV, more than about +24 mV, more than about +25 mV, more than about +26 mV, more than about +27 mV, more than about +28 mV, more than +29 mV. In some embodiments, the nanoparticle disclosed herein has a zeta potential about +5 mV, about +7 mV, about +9 mV, about +10 mV, about +12 mV, about +13 mV, about +14 mV, about +15 mV, about +16 mV, about +17 mV, about +18 mV, about +20 mV, about +22 mV, about +24 mV, about +26 mV, about +28 mV, about +30 mV, about +40 mV, about +41 mV, about +42 mV, about +43 mV, about +44 mV, about +45 mV, about +46 mV, about +47 mV, about +48 mV, about +49 mV, about +50 mV, about +55 mV, about +60 mV, about +70 mV, about +80 mV, about +90 mV, or about +100 mV.

In some embodiments, the about of A01B present in the nanoparticle can be about 0.1% to 0.5%, 0.2% to about 0.4%, about 0.5%, about 1%, about 3%, about 5%, about 10%, about 15%, about 20%, about 50% of its nanoparticle weight.

In some examples, the amount of compound or a pharmaceutically acceptable salt thereof present in the nanoparticle can be from about 0.1%, about 0.2%, about 0.3%, about 0.4%, about 0.5%, about 0.6%, about 0.7%, about 0.8%, about 0.9%, about 1%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 120.5%, about 13%, about 130.5%, about 14%, about 15%, about 16%, about 17%, about 18%, about 19%, about 20%, about 22%, about 24%, about 26%, about 28%, about 30%, about 32%, about 34%, about 36%, about 38%, or about 40% of its nanoparticle weight.

Further, disclosed nanoparticles may be able to efficiently bind to or otherwise associate with a biological entity, for example, a particular membrane component or cell surface receptor on a muscle cell that facilitates delivery into the cell. In some examples, the nanoparticle further comprises a muscle cell-targeting agent. In some examples, the muscle cell-targeting agent is an A01B aptamer. In some embodiments, the A01B aptamer comprises a sequence at least about 60% (e.g., at least about 65%, at least about 70%, at least about 75%, at least about 80%, at least about 85%, at least about 90%, at least about 95%, at least about 98%, or at least about 99%) identical to SEQ ID NO: 5 or a fragment thereof.

In some embodiments, the A01B aptamer comprises at least one chemically modified nucleotide. In some embodiments, the chemically modified nucleotide is selected from 2′-O-methyl (2′-O-Me), 2′-Fluoro (2′-F), 2′-deoxy-2′-fluoro-beta-D-arabino-nucleic acid (2′F-ANA), 4′-S, 4′-SFANA, 2′-azido, UNA, 2′-O-methoxy-ethyl (2′-O-ME), 2′-O-Allyl, 2′-O-Ethylamine, 2′-O-Cyanoethyl, Locked nucleic acid (LAN), Unlocked nucleic acid (UNA), Methylene-cLAN, N-MeO-amino BNA, or N-MeO-aminooxy BNA. In some embodiments, the chemically modified nucleotide is selected from 2′-O-methyl (2′-O-Me) and 2′-Fluoro (2′-F). In some embodiments, the A and G residues of the A0 TB aptamer are modified to 2-O-Met. In some embodiments, the C and U residues are modified to 2′-F. In some embodiments, all the A and G residues of SEQ ID NO: 5 are modified to 2-O-Met and all the C and U residues of SEQ ID NO: 5 are modified to 2′-F.

In some embodiments, hydrogels (for example, thermoreversible gel) can be created for depot delivery of the nanoparticles (e.g., P7C3 nanoparticle). Accordingly, in some embodiments, the nanoparticle is in a nanogel drug composition comprising a nanogel. In some embodiments, the nanogel comprises a thermal reversable nanogel. In some embodiments, the thermal reversable nanogel comprises a methoxy poly (ethylene glycol)-polycaprolactone copolymer. The concentration of the methoxy poly (ethylene glycol)-polycaprolactone copolymer-based nanogel can be, for example, about 10% w/v, about 11% w/v, about 12% w/v, about 13% w/v, about 14% w/v, about 15% w/v, about 16% w/v, about 17% w/v, about 18% w/v, about 19% w/v, about 20% w/v, about 21% w/v, about 22% w/v, about 23% w/v, about 24% w/v, about 25% w/v, about 26% w/v, about 27% w/v, about 28% w/v, about 29% w/v, about 30% w/v, about 31% w/v, about 32% w/v, about 33% w/v, about 34% w/v, about 35% w/v, about 36% w/v, about 37% w/v, about 38% w/v, about 39% w/v, about 40% w/v, about 41% w/v, about 42% w/v, about 43% w/v, about 44% w/v, about 45% w/v, about 46% w/v, about 47% w/v, about 48% w/v, about 49% w/v, about 50% w/v, about 55% w/v, or about 60% w/v.

In some examples, the compositions and methods herein can be used to treat and/or prevent an inflammatory disease including but not limited to muscle injury, including, for example, Hyperlipidemia, fatty liver disease (steatosis), steatohepatitis, metabolic syndrome, Phenylketonuria (PKU), Maple syrup urine disease (MSUD), Gaucher's disease, hypercholesterolemia, hypertriglyceridemia, hyperthyroidism, hypothyroidism, dyslipidemia, hypolipidemia, and galactosemia, alcoholic liver disease (ALD), or non-alcoholic fatty liver disease (such as, for example, non-alcoholic fatty liver (NAFL) or non-alcoholic steatohepatitis (NASH)), liver fibrosis, lung inflammatory disease (such as, for example, acute lung injury, acute respiratory distress syndrome (ARDS), transfusion induced acute lung injury (TRALI), or ventilator induced lung injury), acute inflammation, sepsis, and/or an autoinflammatory disease.

As used herein “autoinflammatory disorders” refer to disorders where the innate immune response attacks host cells. Examples of autoimmune diseases that can be treated by any of the NAD activator disclosed herein include, but are not limited to asthma, graft versus host disease, allergy, transplant rejection, Familial Cold Autoinflammatory Syndrome (FCAS), Muckle-Wells Syndrome (MWS), Neonatal-Onset Multisystem Inflammatory Disease (NOMID) (also known as Chronic Infantile Neurological Cutaneous Articular Syndrome (CINCA)), Familial Mediterranean Fever (FMF), Tumor Necrosis Factor (TNF)—Associated Periodic Syndrome (TRAPS), TNFRSF11A-associated hereditary fever disease (TRAPS11), Hyperimmunoglobulinemia D with Periodic Fever Syndrome (HIDS), Mevalonate Aciduria (MA), Mevalonate Kinase Deficiencies (MKD), Deficiency of Interleukin−1ß (IL-1ß) Receptor Antagonist (DIRA) (also known as Osteomyelitis, Sterile Multifocal with Periostitis Pustulosis), Majeed Syndrome, Chronic Nonbacterial Osteomyelitis (CNO), Early-Onset Inflammatory Bowel Disease, Diverticulitis, Deficiency of Interleukin−36-Receptor Antagonist (DITRA), Familial Psoriasis (PSORS2), Pustular Psoriasis (15), Pyogenic Sterile Arthritis, Pyoderma Gangrenosum, and Acne Syndrome (PAPA), Congenital sideroblastic anemia with immunodeficiency, fevers, and developmental delay (SIFD), Pediatric Granulomatous Arthritis (PGA), Familial Behçets-like Autoinflammatory Syndrome, NLRP12-Associated Periodic Fever Syndrome, Proteasome-associated Autoinflammatory Syndromes (PRAAS), Spondyloenchondrodysplasia with immune dysregulation (SPENCDI), STING-associated vasculopathy with onset in infancy (SAVI), Aicardi-Goutieres syndrome, Acute Febrile Neutrophilic Dermatosis, X-linked familial hemophagocytic lymphohistiocytosis, and Lyn kinase-associated Autoinflammatory Disease (LAID), Achalasia, Acute disseminated encephalomyelitis, Acute motor axonal neuropathy, Addison's disease, Adiposis dolorosa, Adult Still's disease, Agammaglobulinemia, Alopecia areata, Alzheimer's disease, Amyloidosis, Ankylosing spondylitis, Anti-GBM/Anti-TBM nephritis, Antiphospholipid syndrome, Aplastic anemia, Autoimmune angioedema, Autoimmune dysautonomia, Autoimmune encephalomyelitis, Autoimmune enteropathy, Autoimmune hemolytic anemia, Autoimmune hepatitis, Autoimmune inner ear disease (AIED), Autoimmune myocarditis, Autoimmune oophoritis, Autoimmune orchitis, Autoimmune pancreatitis, Autoimmune polyendocrine syndrome, Autoimmune retinopathy, Autoimmune urticaria, Axonal & neuronal neuropathy (AMAN), Baló disease, Behcet's disease, Benign mucosal emphigoid, Bickerstaffs encephalitis, Bullous pemphigoid, Castleman disease (CD), Celiac disease, Chagas disease, Chronic fatigue syndrome, Chronic inflammatory demyelinating polyneuropathy (CIDP), Chronic recurrent multifocal osteomyelitis (CRMO), Churg-Strauss Syndrome (CSS), Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Diabetes mellitus type 1, Discoid lupus, Dressler's syndrome, Endometriosis, Enthesitis, Eosinophilic esophagitis (EoE), Eosinophilic fasciitis, Erythema nodosum, Essential mixed cryoglobulinemia, Evans syndrome, Felty syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's encephalopathy, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Inflamatory Bowel Disease (IBD), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus nephritis, Lupus vasculitis, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Ord's thyroiditis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Rheumatoid vasculitis, Sarcoidosis, Schmidt syndrome, Schnitzler syndrome, Scleritis, Scleroderma, Sjögren's syndrome, Sperm & testicular autoimmunity, Stiff person syndrome (SPS), Subacute bacterial endocarditis (SBE), Susac's syndrome, Sydenham chorea, Sympathetic ophthalmia (SO), Systemic Lupus Erythematosus, Systemic scleroderma, Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Urticaria, Urticarial vasculitis, Uveitis, Vasculitis, Vitiligo, Vogt-Koyanagi-Harada Disease, and Wegener's granulomatosis (or Granulomatosis with Polyangiitis (GPA)).

The compositions, compounds, and/or therapeutic agents disclosed herein have cardioprotective effects. As used herein, “cardioprotective”, “cardioprotective” effect, and the like, can refer to refers to the benefit offered by P7C3 and/or other Nampt activator in terms of decreased incidence and/or propensity to long QT, arrhythmias, myocardial ischemia, ischemia-reperfusion injury, cardiomyopathy, cardiomyocyte death, improved cell survival, decreased insulin resistance in the heart, increased cardiac function (ejection fraction and fractional shortening), valvular diseases, anginas, aneurysms, infarction, arrhythmias of myocardial infarction and non-myocardial ischemia origins, preventing maladaptive remodeling of the heart. The pharmaceutical formulations described herein can include an amount of a Nampt activator or analogue thereof described herein that can be effective to treat or prevent a cardiovascular disease or disorder and/or can be cardioprotective. Cardiovascular diseases that can be treated and/or prevented with a Nampt activator, analogue thereof, or formulation thereof, include, but are not limited to, cardiomyopathy, heart failure, arrhythmia, long QT syndrome, long QTc syndrome, long QRS syndrome, myocardial ischemia, myocardial infarction (MI), arrhythmias of ischemic and non-ischemic origin, inflammation, vascular dysfunction, cardiomyopathy, cardiac remodeling, maladaptation, anginas of different types, drug induced heart failure, iatrogenic heart and vascular diseases, or any combination thereof. The Nampt activator or analogue thereof, in some aspects, can be included in the manufacture of a medicament for treatment of a cardiovascular disease or disorder and/or diabetes in a subject that may or may not have diabetes mellitus or a symptom thereof.

The compositions, compounds, and/or therapeutic agents disclosed herein have cardioprotective effects. As used herein, “cardioprotective”, “cardioprotective” effect, and the like, can refer to refers to the benefit offered by P7C3 and/or other Nampt activator in terms of decreased incidence and/or propensity to long QT, arrhythmias, myocardial ischemia, ischemia-reperfusion injury, cardiomyopathy, cardiomyocyte death, improved cell survival, decreased insulin resistance in the heart, increased cardiac function (ejection fraction and fractional shortening), valvular diseases, anginas, aneurysms, infarction, arrhythmias of myocardial infarction and non-myocardial ischemia origins, preventing maladaptive remodeling of the heart. The pharmaceutical formulations described herein can include an amount of a Nampt activator or analogue thereof described herein that can be effective to treat or prevent a cardiovascular disease or disorder and/or can be cardioprotective. Cardiovascular diseases that can be treated and/or prevented with a Nampt activator, analogue thereof, or formulation thereof, include, but are not limited to, cardiomyopathy, heart failure, arrhythmia, long QT syndrome, long QTc syndrome, long QRS syndrome, myocardial ischemia, myocardial infarction (MI), arrhythmias of ischemic and non-ischemic origin, inflammation, vascular dysfunction, cardiomyopathy, cardiac remodeling, maladaptation, anginas of different types, drug induced heart failure, iatrogenic heart and vascular diseases, or any combination thereof. The Nampt activator or analogue thereof, in some aspects, can be included in the manufacture of a medicament for treatment of a cardiovascular disease or disorder and/or diabetes in a subject that may or may not have diabetes mellitus or a symptom thereof.

The dosage of administration can be from about 0.01 mg/kg body mass to about 1000 mg/kg body mass. In some examples, the dosage is about 0.01 mg/kg body mass, about 0.05 mg/kg body mass, about 0.1 mg/kg body mass, about 0.5 mg/kg body mass, about 1 mg/kg body mass, about 1.5 mg/kg body mass, about 2 mg/kg body mass, about 2.5 mg/kg body mass, about 3 mg/kg body mass, about 3.5 mg/kg body mass, about 4 mg/kg body mass, about 4.5 mg/kg body mass, about 5 mg/kg body mass, about 5.5 mg/kg body mass, about 6 mg/kg body mass, about 6.5 mg/kg body mass, about 7 mg/kg body mass, about 7.5 mg/kg body mass, about 8 mg/kg body mass, about 8.5 mg/kg body mass, about 9 mg/kg body mass, about 9.5 mg/kg body mass, about 10 mg/kg body mass, about 11 mg/kg body mass, about 12 mg/kg body mass, about 13 mg/kg body mass, about 14 mg/kg body mass, about 15 mg/kg body mass, about 20 mg/kg body mass, about 25 mg/kg body mass, about 30 mg/kg body mass, about 35 mg/kg body mass, about 40 mg/kg body mass, about 45 mg/kg body mass, about 50 mg/kg body mass, about 55 mg/kg body mass, about 60 mg/kg body mass, about 65 mg/kg body mass, about 70 mg/kg body mass, about 75 mg/kg body mass, about 80 mg/kg body mass, about 85 mg/kg body mass, about 90 mg/kg body mass, about 95 mg/kg body mass, about 100 mg/kg body mass, about 500 mg/kg body mass, or about 1000 mg/kg body mass. The dosage forms can be adapted for administration by any appropriate route.

Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavemous, intrathecal, intravitreal, intracerebral, gingival, subgingival, intracerebroventricular, and intradermal. Such formulations may be prepared by any method known in the art.

The disclosed methods can be performed any time prior to the onset of the disease. In some aspects, the disclosed methods can be employed 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 years; 12, 11, 10, 9, 8, 7, 6, 5, 4, 3, 2, or 1 months; 30, 29, 28, 27, 26, 25, 24, 23, 22, 21, 20, 19, 18, 17, 16, 15, 14, 13, 12, 11, 10, 9, 8, 7, 6, 5, 4, or 3 days; 60, 48, 36, 30, 24, 18, 15, 12, 10, 9, 8, 7, 6, 5, 4, 3, or 2 hours prior to the onset of the disease; or 1, 2, 3, 4, 5, 6, 7, 8, 9, 10, 15, 20, 25, 30, 35, 40, 45, 50, 55, 60, 75, 90, 105, 120 minutes; 3, 4, 5, 6, 7, 8, 9, 10, 11, 12, 15, 18, 24, 30, 36, 48, 60 hours; 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 or more days; 4, 5, 6, 7, 8, 9, 10, 11, 12 or more months; 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 years after the onset of the disease.

Dosing frequency for the composition of any preceding aspect, includes, but is not limited to, at least once every 12 months, once every 11 months, once every 10 months, once every 9 months, once every 8 months, once every 7 months, once every 6 months, once every 5 months, once every 4 months, once every 3 months, once every two months, once every month; or at least once every three weeks, once every two weeks, once a week, twice a week, three times a week, four times a week, five times a week, six times a week, or daily. In some embodiment, the interval between each administration is less than about 4 months, less than about 3 months, less than about 2 months, less than about a month, less than about 3 weeks, less than about 2 weeks, or less than less than about a week, such as less than about any of 6, 5, 4, 3, 2, or 1 day. In some embodiment, the dosing frequency for the composition includes, but is not limited to, at least once a day, twice a day, or three times a day. In some embodiment, the interval between each administration is less than about 48 hours, 36 hours, 24 hours, 22 hours, 20 hours, 18 hours, 16 hours, 14 hours, 12 hours, 10 hours, 9 hours, 8 hours, or 7 hours. In some embodiment, the interval between each administration is less than about 24 hours, 22 hours, 20 hours, 18 hours, 16 hours, 14 hours, 12 hours, 10 hours, 9 hours, 8 hours, 7 hours, or 6 hours. In some embodiment, the interval between each administration is constant. For example, the administration can be carried out daily, every two days, every three days, every four days, every five days, or weekly. Administration can also be continuous and adjusted to maintaining a level of the compound within any desired and specified range.

In some embodiments, the dosing frequency of the NAD activator (e.g., P7C3, P7C3 A20, or P7C3 S243) or a pharmaceutically acceptable salt thereof that is formulated in the nanoparticle or the nanogel composition disclosed herein is less (e.g., about 2-fold less, about 3-fold less, about 4-fold less, about 5-fold less, about 6-fold less, about 7-fold less, about 8-fold less, about 9-fold less, about 10-fold less, about 15-fold less, about 20-fold less, about 30-fold less, about 40-fold less, or about 50-fold less) than the dosing frequency of the NAD activator (e.g., P7C3, P7C3 A20, or P7C3 S243) when the NAD activator is administered without the nanoparticle or the nanogel composition.

Such amounts of the NAD activator (e.g., P7C3, P7C3 A20, or P7C3 S243) or a pharmaceutically acceptable salt thereof dispersed or encapsulated in the nanoparticle or in the nanogel drug composition disclosed herein can be generally smaller, e.g., at least about 10% smaller, than the amount of the NAD activator (e.g., P7C3, P7C3 A20, or P7C3 S243) or a pharmaceutically acceptable salt thereof present in the current dosage of the treatment regimen (i.e., without nanoparticle or nanogel drug composition) required for producing essentially the same therapeutic effect. Indeed, the NAD activator (e.g., P7C3, P7C3 A20, or P7C3 S243) or a pharmaceutically acceptable salt thereof encapsulated in, or adhered to, a nanoparticle or a nanogel drug composition can potentially increase duration of the therapeutic effect for the NAD activator (e.g., P7C3, P7C3 A20, or P7C3 S243) or a pharmaceutically acceptable salt thereof. Stated another way, encapsulating the NAD activator (e.g., P7C3, P7C3 A20, or P7C3 S243) or a pharmaceutically acceptable salt thereof in a nanoparticle or a nanogel composition or adhering the NAD activator (e.g., P7C3, P7C3 A20, or P7C3 S243) or a pharmaceutically acceptable salt thereof to the nanoparticle or the nanogel composition can increase its therapeutic efficacy, i.e., a smaller amount of the NAD activator (e.g., P7C3, P7C3 A20, or P7C3 S243) or a pharmaceutically acceptable salt thereof encapsulated in a nanoparticle, as compared to the amount present in a typical one dosage administered for a particular muscular disease, can achieve essentially the same therapeutic effect. Accordingly, the nanoparticle or the nanogel drug composition can comprise the NAD activator (e.g., P7C3, P7C3 A20, or P7C3 S243) or a pharmaceutically acceptable salt thereof in an amount which is less than the amount traditionally recommended for one dosage of the NAD activator (e.g., P7C3, P7C3 A20, or P7C3 S243) or a pharmaceutically acceptable salt thereof, while achieving essentially the same therapeutic effect. For example, if the traditionally recommended dosage of the NAD activator (e.g., P7C3, P7C3 A20, or P7C3 S243) or a pharmaceutically acceptable salt thereof is X amount then the nanoparticle or the nanogel drug composition can comprise the NAD activator (e.g., P7C3, P7C3 A20, or P7C3 S243) or a pharmaceutically acceptable salt thereof in an amount of about 0.9×, about 0.8×, about 0.7×, about 0.6×, about 0.5×, about 0.4×, about 0.3×, about 0.2×, about 0.1× or less. Without wishing to be bound by the theory, this can allow administering a lower dosage of the NAD activator (e.g., P7C3, P7C3 A20, or P7C3 S243) or a pharmaceutically acceptable salt thereof in a nanoparticle to obtain a therapeutic effect which is similar to when a higher dosage is administered without the nanoparticle or the nanogel drug composition. Low-dosage administration of the NAD activator (e.g., P7C3, P7C3 A20, or P7C3 S243) or a pharmaceutically acceptable salt thereof can reduce side effects of the NAD activator (e.g., P7C3, P7C3 A20, or P7C3 S243) or a pharmaceutically acceptable salt thereof, if any, and/or reduce likelihood of the subject's resistance to the NAD activator (e.g., P7C3, P7C3 A20, or P7C3 S243) or a pharmaceutically acceptable salt thereof after administration for a period of time.

### EXAMPLES

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific examples of the invention described herein. While the invention has been described with reference to particular examples and implementations, it will be understood that various changes and additional variations may be made and equivalents may be substituted for elements thereof without departing from the scope of the invention or the inventive concept thereof. In addition, many modifications may be made to adapt a particular situation or device to the teachings of the invention without departing from the essential scope thereof. Such equivalents are intended to be encompassed by the following claims. It is intended that the invention not be limited to the particular implementations disclosed herein, but that the invention will include all implementations falling within the scope of the appended claims.

### Example 1. Enhanced Recovery of Muscle Morphology and Function from Strain Injury Through Application of a Small Molecule Activator of NAD Synthesis

This example shows that P7C3 (3,6-Dibromo-a-[(phenylamino)methyl]-9H-carbazole-9-ethanol) targets mechanisms directly related to loss of contractility, in addition to anti-inflammatory actions. The data show that P7C3 acts via mechanisms that can be utilized prophylactically (membrane repair) or post-injury (regeneration of intramuscular nerves, enhanced protein synthesis, reduced proteolysis). P7C3 increases NAD in muscle cells by nicotinamide phosphoribosyltransferase (NAMPT) activation. As NAD enhances expression of SIRT1, which is linked to the membrane repair process, P7C3 can mitigate strain-induced membrane disruption, thus speeding recovery from injury. P7C3 can act via several mechanisms to enhance post-strain injury recovery of muscle. Neuroprotective and neurogenic actions have been attributed to P7C3, indicating the function to promote regeneration of intramuscular axons following strain injury. In addition, the metabolic stimulus provided by P7C3 can promote muscle recovery by providing energy to enhance myofibrillar protein synthesis. This metabolic stimulus can promote lipid synthesis to restore and maintain sarcoplasmic reticulum (SR) integrity, which can reduce Ca2+ leak, which has been linked to age-related loss of contractility. Such leakiness can also activate the calpain family of proteases. Though part of the normal remodeling process, excessive or prolonged calpain activity can impair recovery through an imbalance in protein synthesis and lysis. Indeed, high calpain activity has been observed in muscle atrophy. The study also focuses on these post-injury mechanisms.

The effect of prophylactic P7C3 administration on membrane injury and repair following muscle strain is assessed. Contractile function, SIRT1 expression and membrane injury and repair (via Evans Blue Dye assay) are assessed immediately and at days 7 post-injury in animals that have received P7C3 for 1 week prior to injury and untreated controls. P7C3 can mitigate membrane injury via SIRT1 increase, and thus improve post-injury contractility relative to control.

To compare effects of P7C3 to those of NSAID on neural and muscular contractile deficits, protein synthesis, SR Ca²⁺ leak and calpain activity following muscle strain injury.

### Results

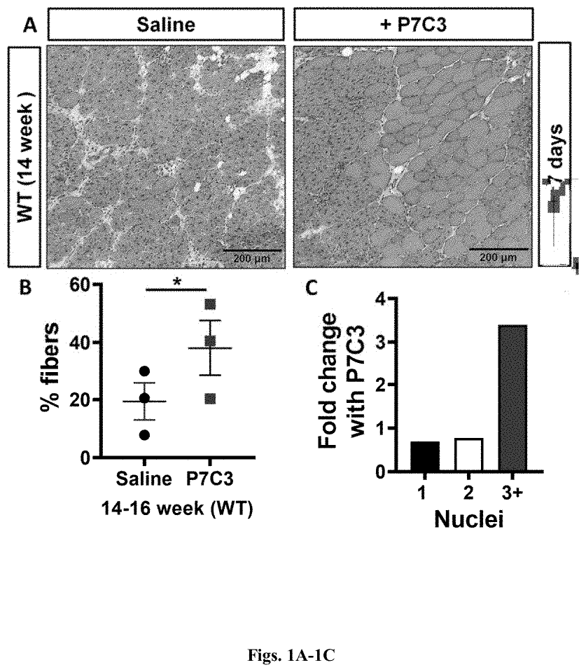

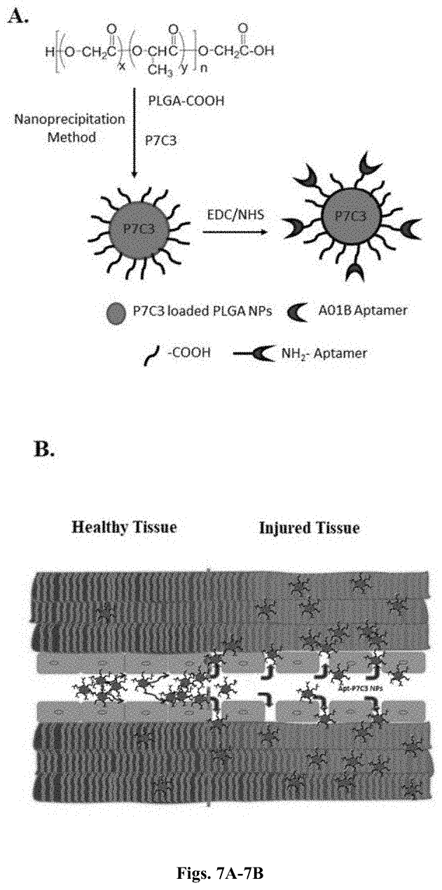

P7C3 administration (1 wk, 5 mg/kg bodyweight/day, i.p.) rescued tibialis anterior muscle injury induced by BaCl₂ in mice. Administration of P7C3 increased frequency of myofiber number and fusion index as noted by increased centrally-nucleated fibers (FIGS. 1A-1C). Overall, these show the use P7C3, and the efficacy of P7C3 as a drug for muscle repair. NAD generation boosted wound closure in C2C12 cells: treatment of C2C12 cells by 1 μM or 100 nM NAMPT activator, P7C3, for 24 hours followed by wire injury significantly augmented gap closure at 16 hrs., showing enhanced membrane repair (FIGS. 7A-7C).

The pre-clinical study involves two experiments. In the first experiment, the primary outcomes are the changes in the number of EBD+ fibers over time and the degree of fibrosis. Secondary outcomes are the rate of myofibrillar protein synthesis and SIRT1 expression. In the second experiment, the primary outcomes are the contractile responses to direct and indirect stimulation and the ratio of direct:indirect stimulated force production and muscle fiber size and centrally-nucleated fibers. Secondary outcomes include expression of NCAM, MyoD, Myogenin.

The study herein shifts the approach to treatment of muscle strain injury away from the traditional anti-inflammatory focus. Although P7C3 has anti-inflammatory actions, it is hypothesized that provision of NAD during injury will promote greater recovery than what is seen with NSAID. The small molecule intervention studied in this example involves a conceptually-different paradigm of targeting the NAD system via NAMPT to enhance energy provision to support recovery in injured muscle. In addition, specific actions of P7C3 (SIRT1) have the potential to either mitigate the extent of muscle strain injury or promote functional recovery.

### Example 2. Method and Uses of P7C3 as Anti-Inflammatory Agent

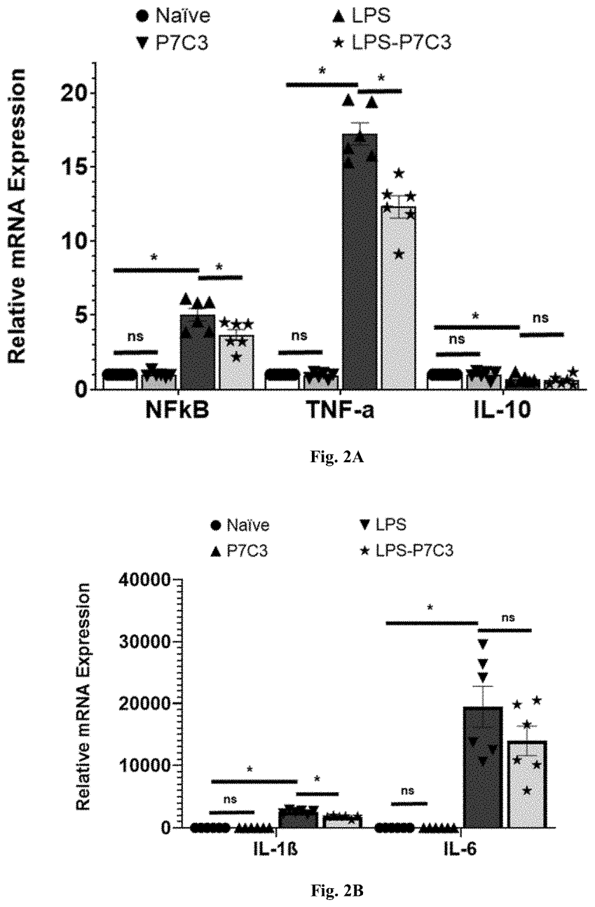

NF-κb is increased with LPS treatment (FIG. 2A, red bar). However, P7C3 treatment (1 μm) significantly decreased the NF-κb expression in RAW264.7 cells. TNFα was also significantly decreased with P7C3 treatment demonstrating that P7C3 is providing anti-inflammatory action in the macrophage cells. IL-10 is protective cytokine and was decreased in both LPS and LPS+P7C3 groups therefore not a major player at least in these cells.

IL-1β was increased with LPS treatment (FIG. 2B, red bar). However, P7C3 treatment (1 μm) significantly decreased the IL-1β expression in RAW264.7 cells. IL-6 was also decreased with P7C3 treatment demonstrating that P7C3 is providing anti-inflammatory action in the macrophage cells.

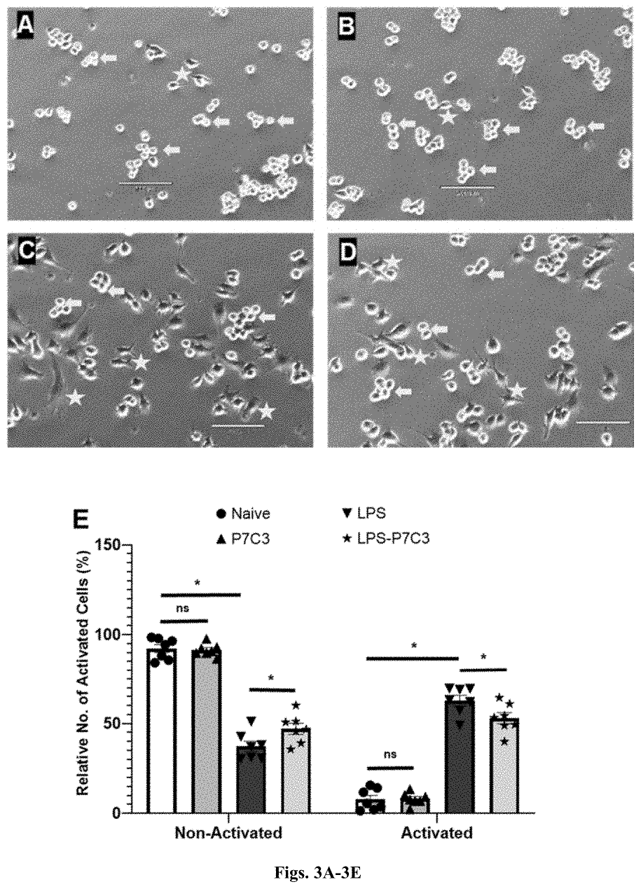

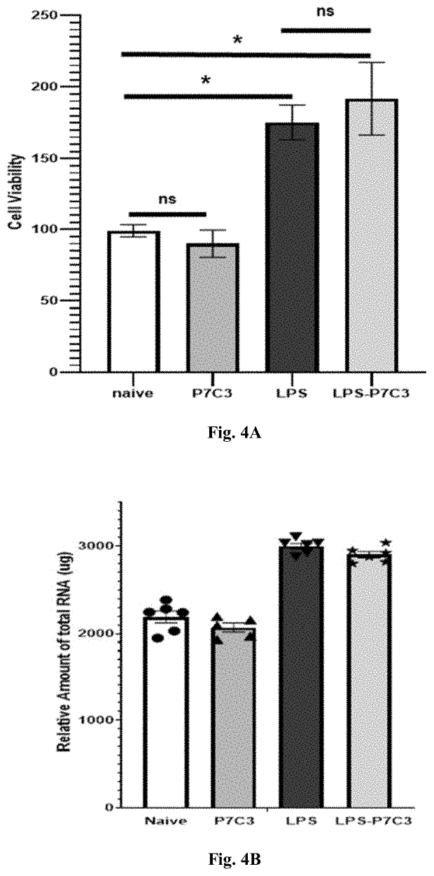

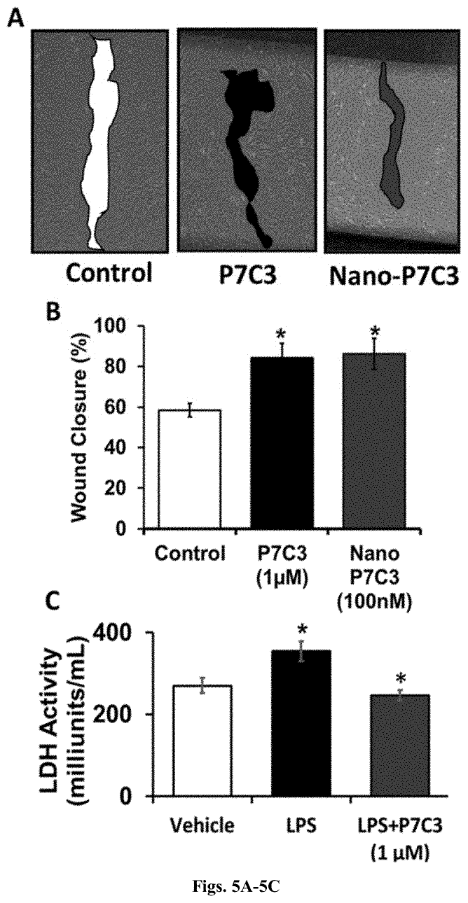

LPS induced activation of RAW264.7 cells depicting inflammation and morphological changes; however, P7C3 treatment significantly decreased the number of activated RAW264.7 cell (FIG. 3E, gray bar). The non-activated RAW264.7 cells are significantly higher compared with LPS group (FIG. 3E, red bar). Overall, both from muscle standpoint as well as from macrophage standpoint P7C3 and related molecules overcome inflammation and provide rescue for skeletal muscle injury and offer novel treatment strategy.

### Example 3. P7C3 Nanoparticles and A01B Skeletal Muscle Specific Aptamer Characterization and Delivery In Vitro

In the present study, the beneficial roles and delivery of Nicotinamide phosphoribosyl transferase (NAMPT) activator P7C3 to skeletal muscle cells was studied. The P7C3 loaded PLGA-COOH NPs were formulated by the nanoprecipitation method. The muscle specific A01B aptamer bioconjugate Poly (D,L-lactic-co-glycolic acid) Carboxylic Acid (PLGA-COOH) nanoparticles (NPs) conjugated with P7C3 were developed and cellular uptake tested. P7C3 loaded A01B aptamer-functionalized NPs demonstrated an encapsulation efficiency of 30.2±2.6%. The particle size 255.9±4.3 nm, polydispersity index of 0.435±0.05 and zeta potential of +10.4±1.8 mV were confirmatory of physical parameters. A sustained in vitro drug release pattern was observed for up to 7 days. A quantitative real time polymerase chain reaction-based evaluation provided confirmation of A01B aptamer binding to the P7C3-Nanoparticles. The in vitro cellular uptake of A01B aptamer functionalized NPs in mouse C2C12 myoblasts demonstrated higher uptake. The cell viability assay showed non-cytotoxic nature of the P7C3 and the functionalized NPs. In vitro wound closure assays showed improved percent closure, with P7C3 NPs. The P7C3 NPs also significantly decreased TNF-α induced NF-κB activity compared with the P7C3 solution in the C2C12/NF-κB reporter cells after 24 h of treatment. Overall, the study demonstrates targeted delivery of P7C3 to the skeletal muscle cells and the formulation can be further developed for anti-inflammatory roles and repair with skeletal muscle injury.

Inflammation is a common basis for major diseases, moreover, aging combined with inflammation (inflamm-aging) leads to accelerated loss of muscle and weakness. Aging and chronic inflammation causes pro-inflammatory cytokines that significantly increases the development of sarcopenia, muscle wasting, and atrophy Aging involves a complicated series of events in which inflammation, senescence, and frailty lead to a significant decrease in quality of life, disease, and death.

P7C3 (3,6-Dibromo-a-[(phenylamino)methyl]-9H-carbazole-9-ethanol), a neuroprotective agent, is an activator of nicotinamide phosphoribosyltransferase (NAMPT), which is a rate limiting enzyme involved in the nicotinamide adenine dinucleotide (NAD) salvage pathway. NAD, a coenzyme present in all living cells, is responsible for the conversion of NAD to NADH, resulting in the production of ATP as an electron carrier. P7C3 offers an increase in NAD/NADH ratio and protects against cell injury. Previous studies demonstrate the efficacy of P7C3 against neuroinflammation, traumatic brain injury, optic nerve injury in addition to neurodegenerative disorders. Moreover, NAMPT has been utilized as a key target for improving diabetes, cardiovascular disease, and aging related disorders, based on this, the efficacy of NAMPT activator P7C3 was tested for in vitro skeletal muscle delivery. Administration of P7C3 to diabetic mouse showed decreased levels of circulating blood glucose and improved skeletal muscle performance. The NADH/NAD ratio is demonstrated to be increased in heart diseases and reported that physiological levels of NADH/NAD can alter function of the heart. The proposed work for the first time demonstrates the efficacy of P7C3 against skeletal muscle injury.

The poly (D,L-lactic-co-glycolic acid) nanoparticles (PLGA NPs) are used as a carrier for sustained delivery of P7C3 for treatment of skeletal muscle injury. The NPs are biocompatible, biodegradable and FDA approved which has potential for transition into clinical trials. The PLGA is non-toxic and degrades in body via hydrolysis pathway resulting in the by-products of glycolic acid and lactic acid monomers similar to various metabolic pathways of body under normal physiological condition. The PLGA-NPs shown controlled drug release profile with established safety in clinical trials. The advantages of using nanotechnology for muscle targeted drug delivery are easy alteration of NPs to get higher sustained release effect and higher accumulation of NPs in injured tissues via enhanced permeation and retention (EPR) mechanism due to enhance vascular permeability.

The goal was to develop PLGA-COOH NPs surface functionalized with muscle specific A01B aptamer for targeted delivery to the muscle cells. Aptamers, DNA, or RNA oligonucleotides binds with target antigen through intermolecular interactions with high specificity and affinity similar to antibodies. The aptamers are non-immunogenic, and stable in a wide pH range, temperature, and organic solvents. Aptamers are synthesized by chemical process with less batch-to-batch variation and does not rely on biological systems compared to antibodies as targeting ligands. The targeted delivery of P7C3 to the skeletal muscle cells was tested using PLGA-COOH NPs functionalized with aptamer A01B, which provides higher efficacy, and reduction in dose and toxicity.

In this proposed work, the P7C3 loaded PLGA-COOH NPs conjugated with A01B-NH2 aptamer were developed and characterized for targeted muscle specific delivery (FIG. 7). The NPs were characterised for their particle size, zeta potential, morphology, encapsulation efficiency, drug loading, in vitro drug release, cell viability and cellular uptake. To evaluate this, the in vitro anti-inflammatory property, injury model and NF-κB expression assays were performed using mouse C2C12 skeletal myoblast cell cultures treated with targeted NPs of P7C3 and cell recovery was monitored.

### Materials and Methods

Materials. P7C3 was ordered from Cayman Chemical Company, Ann Arbor, Mich., USA. Lactate Dehydrogenase (LDH) Activity Assay Kit and dialysis tubing cellulose membrane (flat width: 10 mm; MWCO 14 KDa) were acquired from Sigma-Aldrich (St. Louis, Mo., USA). PLGA-COOH (with 50:50 co-polymer ratio and MW 20 KDa) was obtained from Akina Inc., West Lafayette, Ind., USA. Nucleus stain DAPI (4′,6-diamidino-2-phenylindole) 3-(4,5-dimethylthiazol-2-yl)-2,5-diphenyltetrazolium bromide salt (MTT dye) was obtained from Thermo Fisher Scientific (Lansing, Mich., USA). Phosphate buffered saline (PBS) was purchased from Mediatech, Inc. (Manassas, Va., USA). NBD-cholesterol dye ((22-(N-(7-Nitrobenz-2-Oxa-1,3-Diazol-4-yl)Amino)-23,24-Bisnor-5-Cholen−3β-O1) was also purchased from Invitrogen™ Thermo Fisher Scientific, Waltham, Mass., USA. Cell Mask™ deep red plasma stain was purchased from Molecular Probes, Invitrogen™ Thermo Fisher Scientific, USA. The mouse C2C12 myoblast cells and DMEM F12 (Dulbecco's Modification of Eagle's Medium F 12) (ATCC® 30-2006™) was acquired from the American Type Culture Collection (ATCC) (VA, USA). C2C12/NF-κB reporter cells were bought from Cellomics Technology (Halethorpe, Md., USA). Trypsin EDTA (0.05%) was bought from Thermo Fisher Scientific (Lansing, Mich., USA) and Penicillin-streptomycin (10,000 U/ml) as well as Fetal bovine serum (FBS) were obtained from Gibco Thermo Fisher Scientific, USA. Phosphate Buffer Saline (PBS) for cell culture use was purchased from Corning Cellgro (Manassas, Va., USA). Reporter lysis buffer and luciferase assay reagent were received by Promega (Madison, Wis., USA). Puromycin was acquired from Invitrogen (San Diego, Calif., USA). TNF-α was bought from R&D Systems (Minneapolis, Minn., USA). Pierce™ 660 nM Protein Assay Reagent and Pierce™ Bovine Serum Albumin Standards were procured from Thermo Fisher Scientific (Waltham, Mass., USA). No added purification process was carried out for other analytical reagent grade chemicals, and they were used as they were obtained. Acetone and methanol were purchased from VWR International, Batavia, Ill., USA. All other chemicals used in the study were of analytical grade and were used without any further purification.

Cell culture. C2C12, mouse myoblast cell line cell line (ATCC® CRL-1772™) cells were allowed to grow in DMEM F12 medium supplemented with 1% penicillin-streptomycin antibiotics and 10% v/v FBS. The cell cultures were allowed to maintain and incubated at temperature of 37° C., in a humidified atmosphere with 5% CO₂ in the incubator.

Preparation of P7C3 Nanoparticles. The NPs were prepared using PLGA-COOH polymer (50:50 DLG, MW 20 KDa, Akina Inc., USA) using our previously reported nanoprecipitation method. Briefly, 2.8 mg of P7C3 was dissolved in 1 ml acetone and 14 mg of PLGA-COOH was subsequently added to the acetone. The resultant organic solution was drop-wise added to RNase-free water (2 mL) formerly adjusted to a pH of 9.0 using sodium hydroxide with continuous stirring at 350 rpm, which will allow formation of NPs. The NPs were allowed to stir for overnight to allow complete evaporation of acetone and the next day the NPs were collected after centrifuging them at 5000 rpm for 20 min at room temperature. The NPs pellet was resuspended in 3 ml distilled water. The final pH of the P7C3 NPs was found to be 5.0±0.1. Placebo NPs and NBD-cholesterol dye loaded NPs (by loading dye instead of drug for confocal study) were formulated using similar procedure.

Conjugation of the aptamer with NPs. The P7C3 loaded PLGA-COOH NPs in DNase RNase-free water (2.4 μg/μl) was allowed to incubate with 100 mM N-hydroxysuccinimide and 400 mM 1-ethyl-3-(3-dimethylaminopropyl)-carbodiimide at room temperature for 15 min with gentle stirring. The resulting N-hydroxysuccinimide-activated particles were covalently linked to 5′-NH2 modified A01B aptamer (MW 28,208.86) (2% weight compared with polymer). The resulting Aptamer conjugated NPs were washed, resuspended in PBS, and used immediately.

Determination of Particle Size and Zeta Potential. The dynamic light scattering technique (DLS) was utilized to detect particle size and polydispersity index (PDI) of synthesized NPs and Nano ZS90 (Malvern Instruments Ltd., UK, Zeta Sizer Software Ver. 7.10) instrument was used at 90° scattering angle with 633 nm He—Ne laser light source. The NPs suspension was diluted to 10 times using double distilled water and the size analysis was performed at 25° C. in triplicate. The particle size of NPs was reported as Z-average and polydispersity index (PDI).

Zeta potential of formulated NPs was determined using the same instrument Nano ZS90 based on Smoluchowski equation, which reflects electrophoretic mobility of the NPs and their backscatter at 90°. The NPs suspension was diluted 10 times with double distilled water before the analysis and the sample was read in triplicate.

Transmission Electron Microscopy (TEM). Transmission electron microscope (JEOL JEM 1400 electron microscope and Gaton camera, Peabody, Mass., USA) was used to examine morphology, shape, size, and physical integrity of developed NPs. Sufficient volume of NPs suspension was taken on EMS formvar support film square grid, 200 Cu and allowed to air dry for 10 min. After that, it was negatively stained using 2% w/v phosphotungstic acid and were visualized at accelerating voltage of about 120 kV with ×40,000 magnifications.

Encapsulation Efficiency (EE) and Drug Loading Capacity (DL) After centrifugation of prepared NPs at 5000 rpm for 20 min, the NPs pellet was washed three times with water with the aim to remove any unentrapped free drug, or polymer and suspended in saline. The NPs were treated with methanol (10 times) to extract the loaded P7C3 drug and was quantified using UV spectroscopy (S-2150UV; Cole Parmer Instrument Company) at wavelength of 240 nm (λmax) in methanol (Y=0.0119x+2.8227 and R2=0.9998). The analytical method was validated for its accuracy and specificity. A recovery spiked study of P7C3 in blank NPs dispersion was conducted using UV spectroscopy at 240 nm. The study was performed with three different spiked concentrations (80%, 100% and 120% of the labeled strength of P7C3 in NPs dispersion).

$$\% Entrapment efficiency = \frac{Actual amount of drug loaded in nanoparticles}{Actual amount of drug used for nanoparticles perparation} \times 100$$

$$\% Loading Efficiency = \frac{Amount of drug in nanoparticles}{Total amount of nanoparticles} \times 100$$

In vitro Drug Release. Membrane dialysis method was used to investigate the drug release profile of P7C3 from the NPs. A 0.5 mL sample volume of aptamer conjugated, and non-conjugated P7C3 NPs was filled in dialysis cassettes (MWCO 14 KDa) and placed in 50 mL of PBS (pH 7.4) release medium with stirring at 100 rpm and temperature of 37° C. At predetermined time intervals, 1 mL sample volume of release media was removed, and the release study was continued for 7 days. The withdrawn sample was analyzed for P7C3 concentration using by UV spectroscopy (Model: S-2150UV; Cole Parmer Instrument Company) at wavelength of 240 nm and UV absorbance of standard dilutions of aqueous P7C3 drug (r²=0.9974) was used as a standard calibration curve to quantify. In vitro drug release study was carried out for each sample in triplicate and results were designated as cumulative quantity of drug released with time.

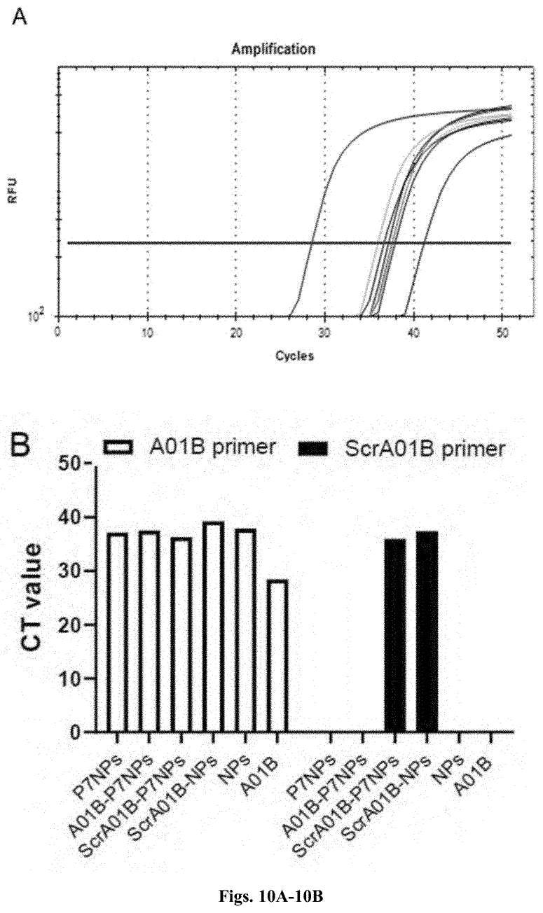

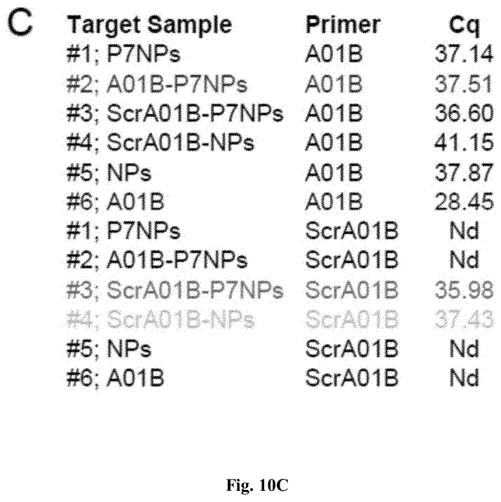

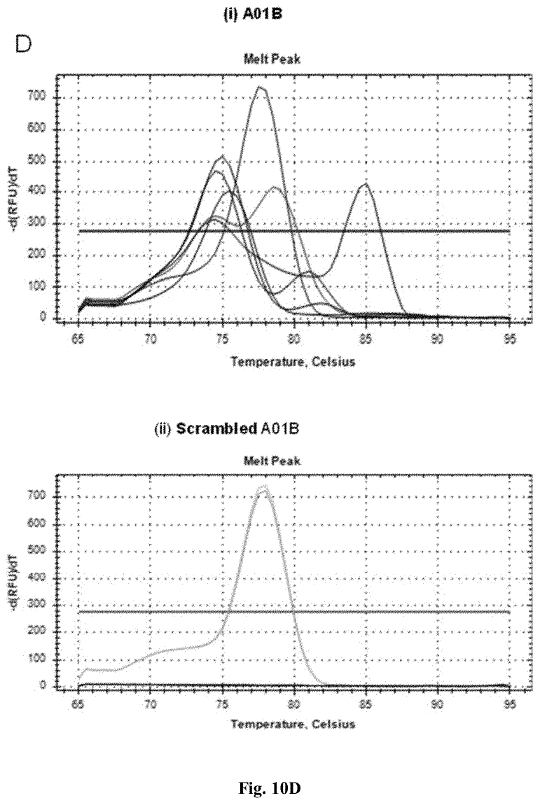

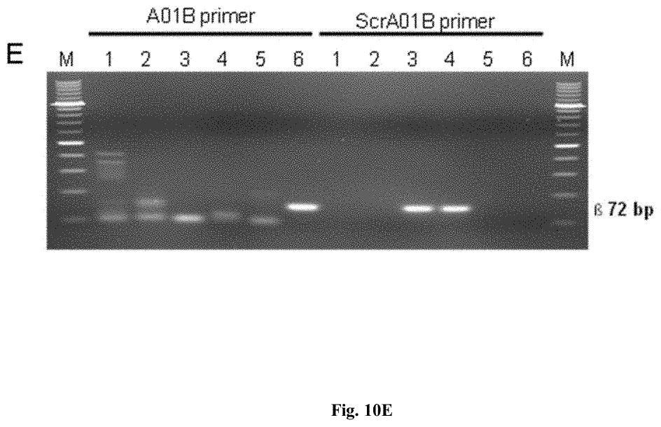

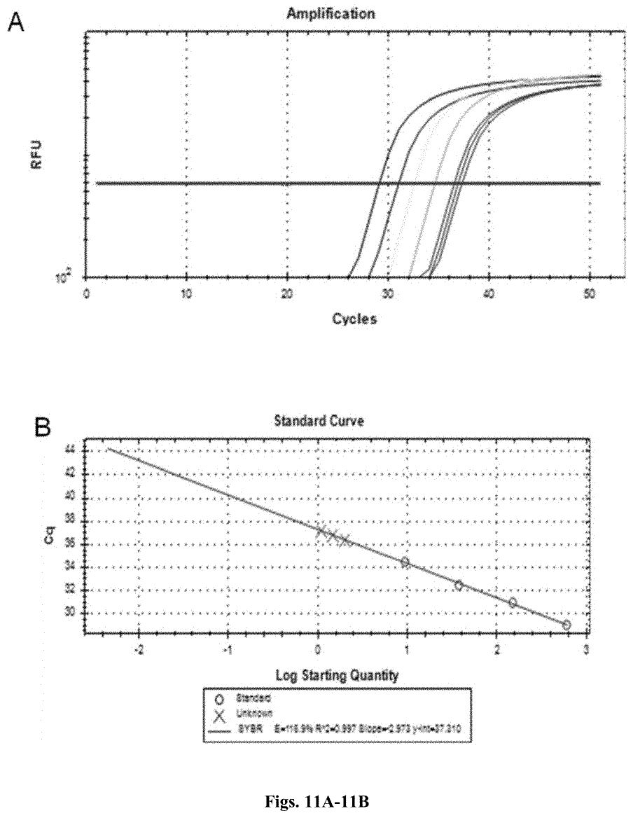

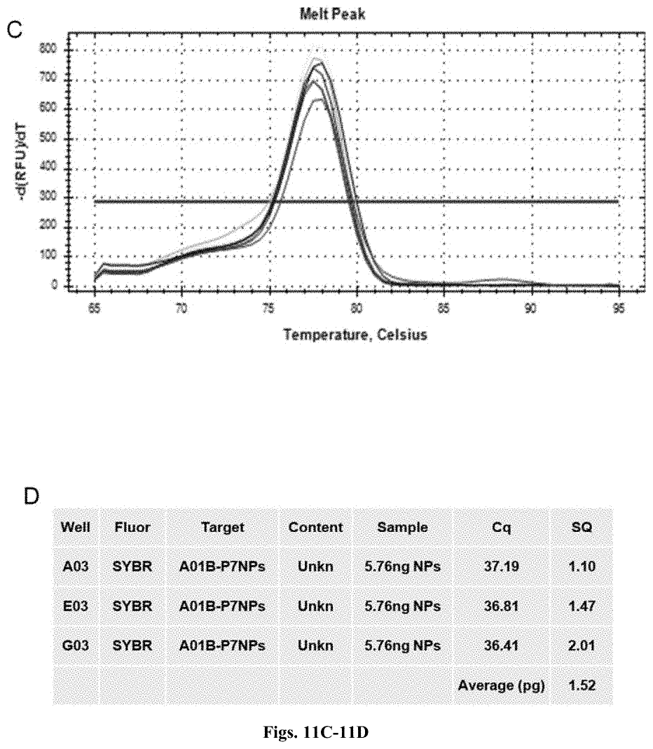

Detection and quantification of A01B aptamer using Real-Time quantitative polymerase chain reaction (RT-qPCR). The aptamer for A01B or Scrambled A01B was detected in nanoparticle-conjugated A01B aptamer encapsulated P7C3 solution using RT-qPCR with A01B- or scrambled A01B-specific primers in FIGS. 10 and 11. The expected amplicon (72 bp) with A01B primer was detected in A01B aptamer containing solution as in FIGS. 10A, 10B, 10C(i), and 10D. With scrambled A01B-specific primers, the amplicon was detected only in scrambled A01B aptamer conjugated nanoparticle solution as in FIGS. 10A, 10B, 10C(ii), and 10D. The amount of A01B aptamer was calculated using standard curve, and 2.6 μg of A01B aptamer conjugated per 1 mg of nanoparticle was measured in A01B-P7 NPs solution in FIG. 11. The percentage of A01B aptamer conjugation to nanoparticles was found to be 0.26%.

Cytotoxicity study. The cytotoxicity of P7C3 nanoparticles (P7C3 NP), and the skeletal muscle-specific aptamer-based targeted P7C3 nanoparticles (P7C3 NP Apt) were determined using the MTT assay in C2C12 cells and was compared with the P7C3 solution, DMSO and control media. Briefly, the mouse C2C12 myoblasts were seeded at a seeding density of 10,000 cells/well in three 96-well plates (Corning, N.Y., USA) in 100 μl of growth medium, i.e., DMEM F12 supplemented with 10% FBS, and 1% P/S and were allowed to grow for 24, 48, and 72 h after an initial attachment period of 24 h, which was considered as time 0. At time 0, the cells were treated with P7C3, P7C3 NP, and P7C3 NP Apt at 0, 10, 500, and 1000 nM concentrations, and was incubated at 37° C., 5% CO₂. The 0 nM concentration had the solvent vehicle control DMSO, in which the drugs P7C3, 7C3 NPs, and P7C3 NPs Apt were dissolved. The MTT reagent solution (1 mg/mL) was added to each well after 24, 48, and 72 h of cell proliferation and the assay was performed according to the manufacturer's protocol (Thermo Fisher Scientific CyQUANT™ MTT Cell Viability Assay kit, USA). The spectrophotometer reading was measured at 540 nm wavelength using a Synergy H1 Hybrid Multi-Mode Reader. Cell viability was calculated and plotted, considering the optical density of solvent vehicle control DMSO wells as 100% viable.

Cellular uptake using confocal microscope. To access the cell uptake potential of the NPs and for determining the localization in cell, confocal microscopy was carried out using C2C12 cells. The fluorescent dye NBD-cholesterol loaded non-targeted PLGA-COOH NPs were prepared as outlined above in the preparation of NPs section. Targeted dye loaded NPs were surface conjugated with A01B aptamer as described previously. C2C12 cells were seeded at a density of 200,000 cells per well in Lab-Tek II 4-chamber Cover Glass System and incubated for a day to achieve 70% confluency for 24 h. Cells were incubated with 1× solution of CellMask membrane stain and a 300 nM solution of DAPI nuclear stain (both diluted in sterile 1×PBS. The excess stain was removed from each chamber, and the adherent cells were once again washed thrice with 1×PBS. Subsequently, 0.5 mL of the dye was loaded and the control NPs treatments were diluted to a concentration of 100 μg/mL in DMEM F12 media were added to the corresponding treatment chambers and allowed to incubate at 22° C. The cells were then examined under confocal microscope FV1200 (Olympus, Tokyo, Japan) at ×60 magnification. The wavelength (in nm) for DAPI (blue), ex: 405 and em: 461, Alexa 488 (green) ex: 488, em: 519 and Alexa 633 (red), ex: 632 and em: 647.

Scratch assay in C2C12 cells. C2C12 cells were seeded into either 6-well or 12-well plates and allowed to grow up to 90% confluence. Scratch or wound injury was induced by streaking the plate (2-3 streaks/well) using a 1000 μL sterilized pipette tip in each well. Treatments were performed with the vehicle or P7C3 groups within the cell culture media. Images were taken of each streak immediately after with an Echo Rebel microscope, and the cell culture plates were then placed in the incubator for 16 h. After 16 h, images were obtained at the same location of the streaks utilizing the coordinates marked. The ImageJ software (NIH, USA) was utilized to calculate the areas of the scratches at the 0 h and 16 h time point, with the area at 16 h divided by the area at 0 h to determine the percent wound closure.

Three sets of experiments were performed with this assay to determine the percent wound closure with the P7C3 solution, the P7C3 NPs, the A01B conjugate P7C3 NPs, and the P7C3 scrambled aptamer NPs. For assessing the effects of the P7C3 solution on percent wound closure, the cells were treated with the vehicle control (DMSO) and 100 nM-2 μM P7C3. For determining the effect of P7C3 NPs, the cells were treated with the vehicle control (blank NPs) and 10 nM-1 μM P7C3 NPs. Finally, for the targeted A01B conjugated P7C3 NPs and scrambled aptamer conjugated P7C3 NPs, the cells were treated with the vehicle controls (blank aptamer conjugated NPs) and 100 nM each of both aptamers conjugated NPs.

Cell culture and Luciferase assay. The C2C12/NF-κB reporter cells were grown in DMEM supplemented with 10% FBS and 1% penicillin/streptomycin. Puromycin selection of the cells was performed by adding 2 μg/mL to the media, and the C2C12 cells with the NF-κB reporter gene were selected for further culturing. Cells were plated at 40,000 cells per well for the initial seeding density using a 24-well format. To induce inflammation, TNF-α was added at a concentration of 10 ng/mL, and simultaneously, the cells were treated with the vehicle control (DMSO or blank NPs), P7C3 solution, and P7C3 NPs. The concentrations of P7C3 and P7C3 NPs added were 100 nM, 500 nM, and 1 μM. After 24 hours, the luciferase assay was conducted per manufacturer protocol (Promega Luciferase Assay System, USA). Using a Synergy H1 Hybrid Multi-Mode Reader, the luminescence of the cell lysate in the opaque 96-well plate was measured. Protein in the cell lysates was measured using a Pierce™ 660 nm protein assay. The luciferase activity was calculated after normalization with protein concentration.

Statistical analysis. Statistical analyses were performed using unpaired student t-test for two groups or with a one-way ANOVA for multiple group comparisons. Values of p≤0.05 was considered as statistically significant. In each experiment group the mean represents n=3-6 and the data are reported as means+SD or +SEM.

### Results

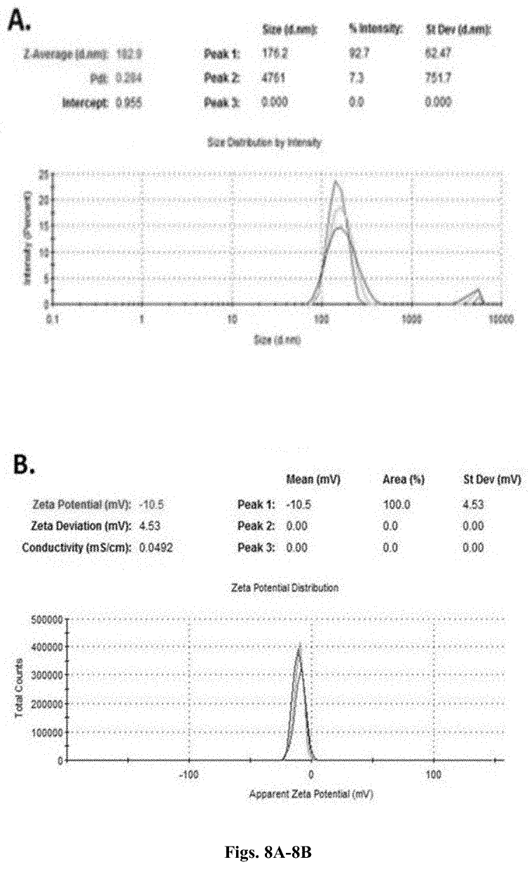

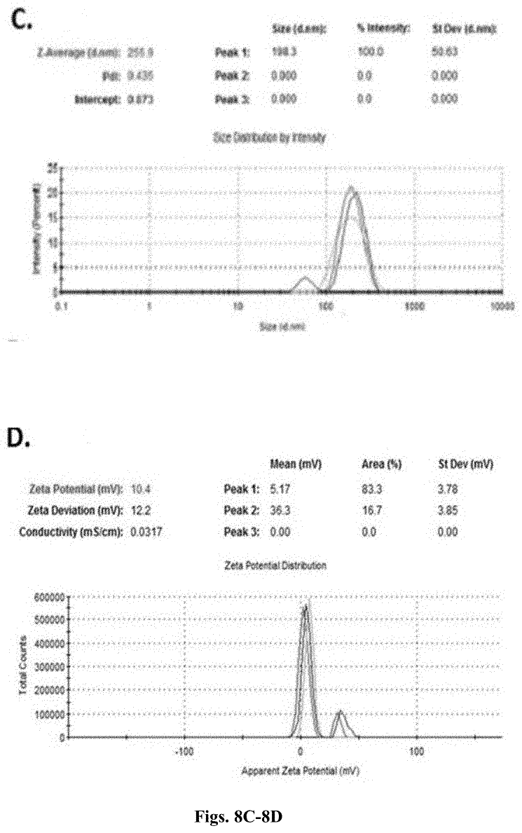

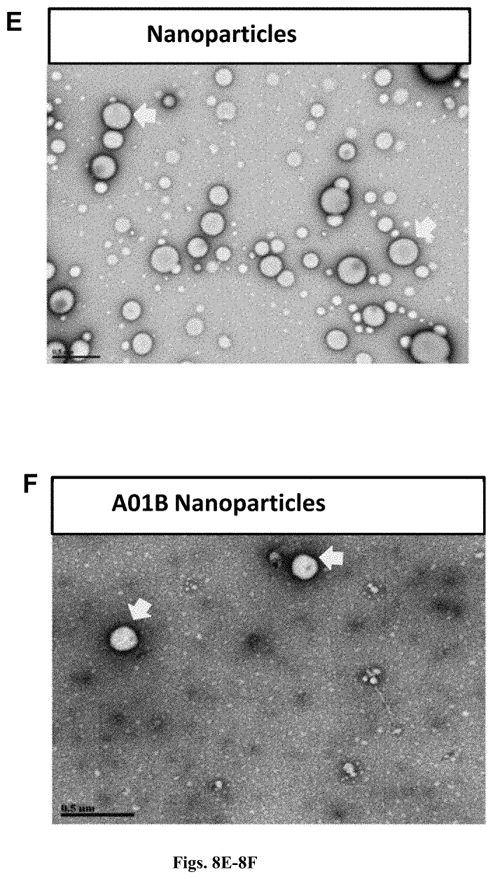

The Nanoparticles size (DLS and TEM) and Zeta potential. The size and polydispersity index (PDI) of P7C3 loaded PLGA-COOH NPs (FIG. 7A) were measured using the dynamic light scattering technique (DLS) technique. The observed size of the non-conjugated NPs loaded with P7C3 was around 182.9±6.3 nm, and with PDI of 0.284±0.03 (FIG. 8A). Whereas, the aptamer conjugated P7C3 NPs was 255.9±4.5 nm with a PDI of 0.435±0.05 (FIG. 8C). The low PDI of the P7C3 loaded exhibited the dispersion homogeneity and uniform distribution of the particle size of the NPs. As shown in FIG. 7B the permeation of PLGA-COOH NPs P7C3 was visualized in the schematic diagram for muscle cells. The mean zeta potential of the unconjugated and aptamer-conjugated P7C3 loaded PLGA-COOH NPs was determined using a Malvern Zetasizer Nano ZS90, and was found to be −10.5±2.1 mV (FIG. 8B), and 10.4±1.8 mV (FIG. 8D), respectively. Subsequent, transmission electron microscopy (TEM) image analysis of the unconjugated (FIG. 8E), and aptamer-conjugated P7C3 NPs (FIG. 8F) displayed that the particle sizes were in the range of 130-220 nm for both the NPs. This indicates the actual size of NPs as contrasting to the size found using DLS technique (zetasizer) that measures the hydrodynamic size of particles. The TEM images also demonstrated both the NPs were smooth and spherical in shape and uniformly distributed and the aptamer conjugation did not affect the morphology of the NPs. Together, these results show that the NPs particles size distribution and characteristics demonstrate uniform particles in the formulations for both the unconjugated and the aptamer-based nanoparticles.

Entrapment efficiency. NPs were centrifuged and the pellets were collected for calculating the entrapment efficiency. Entrapped P7C3 drug was extracted from NPs using methanol and encapsulation efficiency of P7C3 in NPs was determined using UV spectroscopy after generating calibration plot of P7C3 in methanol (r²=0.9998) at 240 nm (λmax). The percent recovery of P7C3 for all concentrations was found to be 100±7% and provides specificity of the analytical method utilized. The entrapment efficiency and percent drug loading were found to be 30.2±2.6% and 5±0.4%, respectively.

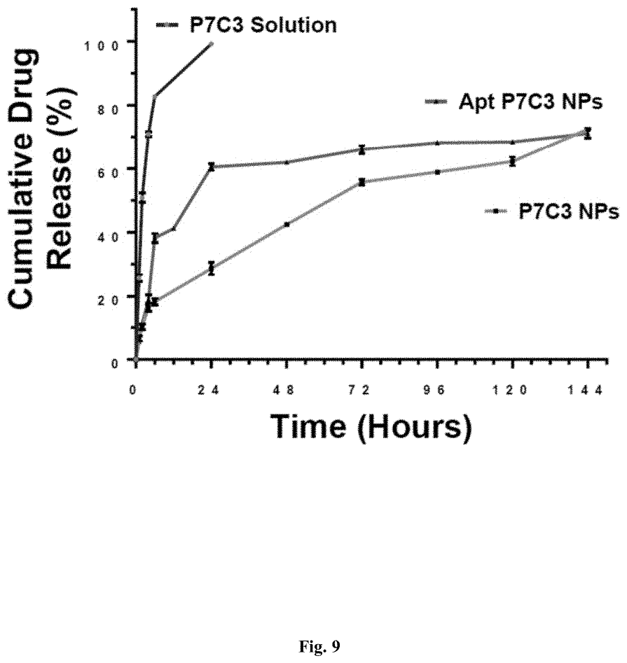

In vitro drug release study. The in vitro drug release study of P7C3 from the solution, non-conjugated NPs and aptamer-conjugated NPs was performed using dialysis method at 37° C.±2° C. for 7 days with PBS (pH 7.4) as the release medium. 100% of the bound P7C3 was found to be released quickly into the release medium in the initial 24 h from the P7C3 solution. Noticeably, the release of P7C3 from the non-conjugated NPs showed a sustained release pattern with 28.7±4.10% release at the end of day 1, followed by continued slow release and showed 72.5±0.9% release of drug at the end of 7 days study in release media. Similarly, the aptamer-conjugated P7C3 NPs also showed sustained drug release behavior with 60.46±1.8% at day 1 and 71.14±2.6% at day 7 (FIG. 9).

A01B aptamer's conjugation efficiency to nanoparticles. The muscle-specific aptamer A01B, and the scrambled A01B was identified in the nanoparticle-conjugated A01B aptamer encapsulated P7C3 solutions using RT-qPCR with A01B- or scrambled A01B-specific primers as shown in FIGS. 10A-10E. The amount of A01B aptamer was calculated using standard curve, and 2.6 μg of A01B aptamer per 1 mg of nanoparticle was measured in A01B-P7 NPs solution as shown in FIGS. 11A-11D. The efficiency of the conjugation of A01B aptamer to nanoparticles was found to be at 2.6/20×100=13.0%.

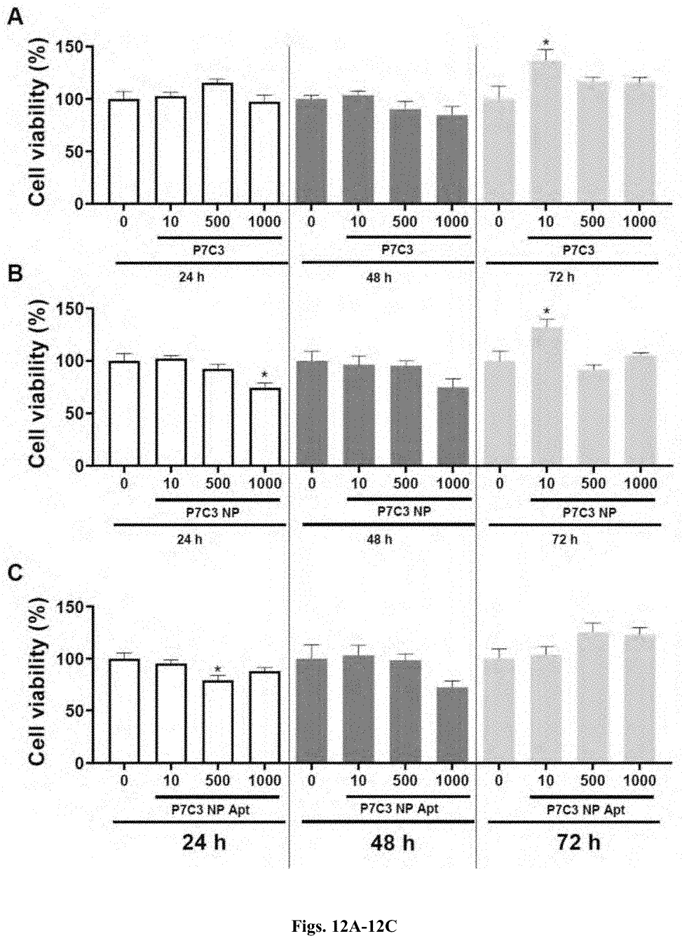

Cytotoxicity study. The cytotoxicity of the P7C3, P7C3 PLGA-COOH NPs (P7C3 NP), and the skeletal muscle-specific aptamer A01B targeted P7C3 PLGA-COOH (P7C3 NP Apt) was studied using the MTT assay in mouse C2C12 myoblasts (FIGS. 12A-12C). Cells were treated with various concentrations (0, 10, 500, and 1000 nM) of P7C3, P7C3 NP, and P7C3 NP Apt for 24, 48, and 72 h. The cell viability was compared to that of the solvent vehicle control DMSO (0 nM). FIGS. 12A-12C indicated that the viability of cells was not affected at the time points analyzed, and at different concentrations except for some higher concentrations at 24 h for P7C3 NP (1000 nM) and P7C3 NP Apt (500 nM), which was found to be non-significant at 48 and 72 h. Interestingly, the cell viability was found to be significantly higher at lower concentration (10 nM) in P7C3 and P7C3 NP treated cells as compared to the control cells at 72 h. There was also an increase in cell viability at 72 h in the P7C3 NP Apt treated cells at 500 and 1000 nM compared to control cells, and was found to be non-significant, where p=0.068 and 0.065, respectively. Taken together this shows that there is no cytotoxic effect of all three P7C3 drug formulations at the time points analyzed. Furthermore, there was also an increase in cell viability at 72 h at lower concentration (10 nM) for P7C3 and P7C3 NP treated cells, and at higher concentrations (500 and 1000 nM) for P7C3 NP Apt treated cells due to an increase in cell proliferation and thereby increased mitochondrial activity in the dye reduction test.

Cellular uptake using confocal microscope. NBD-cholesterol was loaded in to the PLGA-COOH NPs to evaluate the uptake of NPs upon incubation at 37° C. in C2C12 cells. NBD-cholesterol, a hydrophobic dye, was loaded in PLGA-COOH NPs using the same procedure which was optimized to prepare P7C3 NPs by entrapping NBD-cholesterol instead of P7C3 drug. FIG. 13A shows the uptake of NBD-cholesterol PLGA-COOH NPs into C2C12 cells within 1 h of time frame at 37° C. As shown in FIG. 13B, the cellular uptake of NPs increased with an increase in the incubation time for the muscle-specific aptamer A01B-conjugated NPs compared with the scrambled aptamer conjugated NPs, as shown in images taken every 15 min after the treatment and up to 1 hr (FIG. 13A). The relative mean fluorescence intensity was plotted against time (FIG. 13B) for different time points (p<0.05).

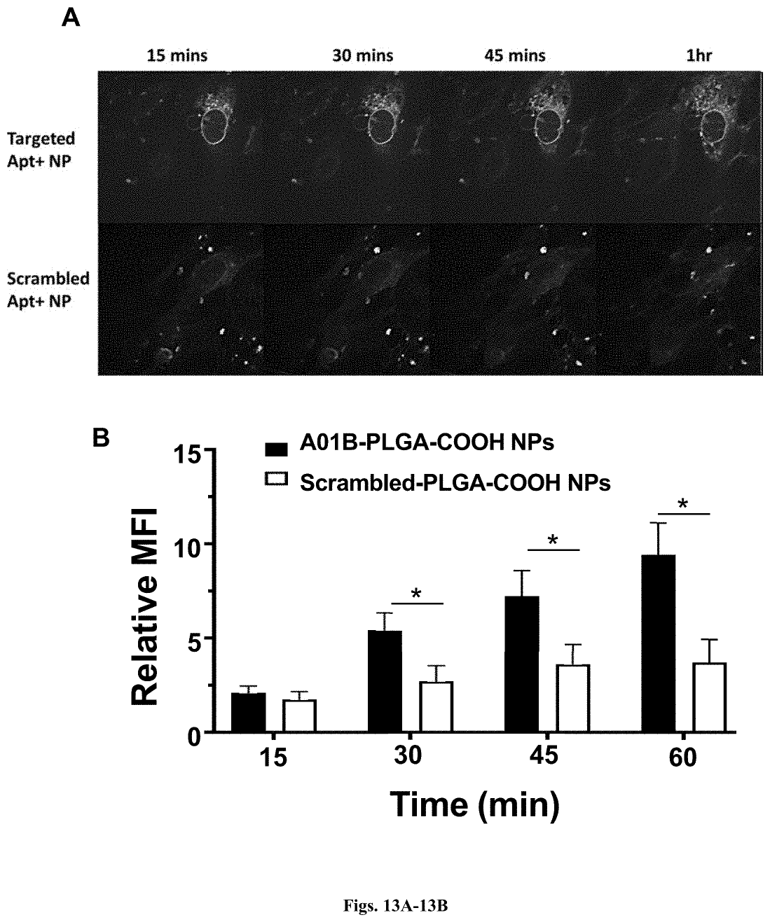

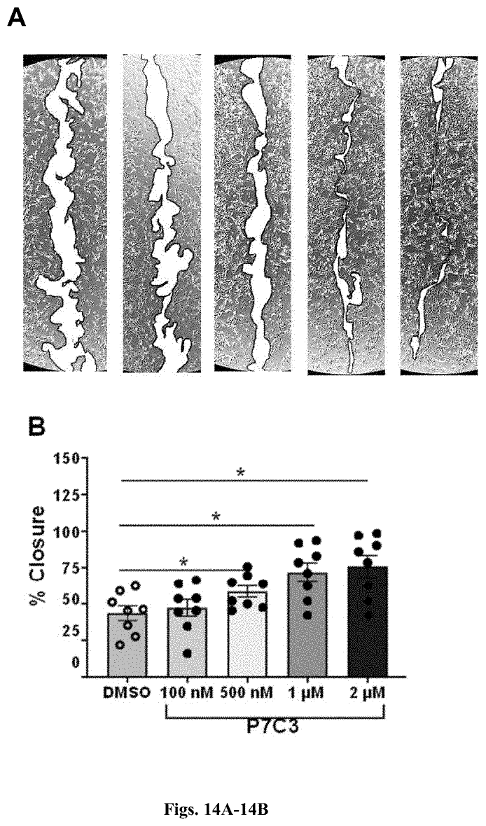

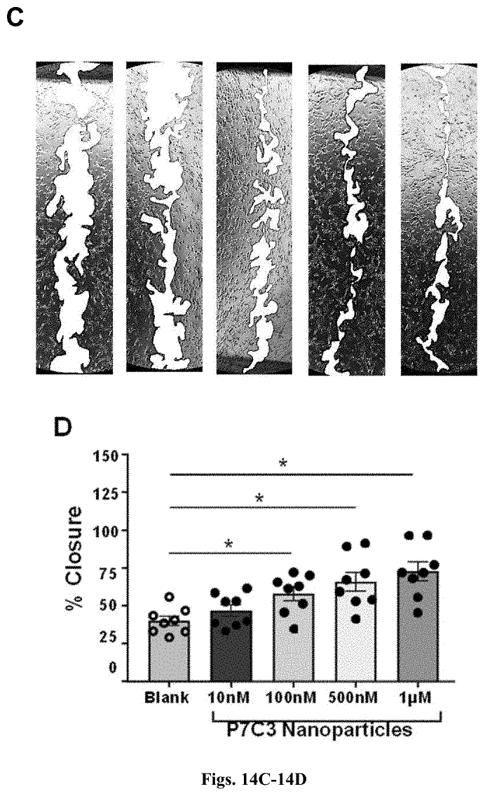

Scratch assay in C2C12 cells. As shown in FIGS. 14A-14D the percent wound closure is significantly increased with 500 nM, 1 and 2 μM P7C3 solutions, and at 100 and 500 nM, and 1 μM concentrations in P7C3 NPs treated cells compared with control cells. The percent wound closure was found to be not significant at 10 and 100 nM concentrations in P7C3 and P7C3 NPs treated cells compared with the control cells, respectively. Furthermore, at an increased 2 μM concentration, the P7C3 treated cells also displayed a significant increase in the percent wound closure compared with the control cells (FIGS. 14A and 14B). The targeted A01B aptamer conjugated P7C3 NPs and scrambled aptamer P7C3 NPs did not demonstrate any significant increase in wound closure within the 16 h time point as compared with the control cells (FIGS. 14E and 14F). Together, the P7C3 and P7C3 NPs display an effective wound closure in treating the muscle cells and provides increased benefits for the body as lower concentrations of the chemicals are needed.

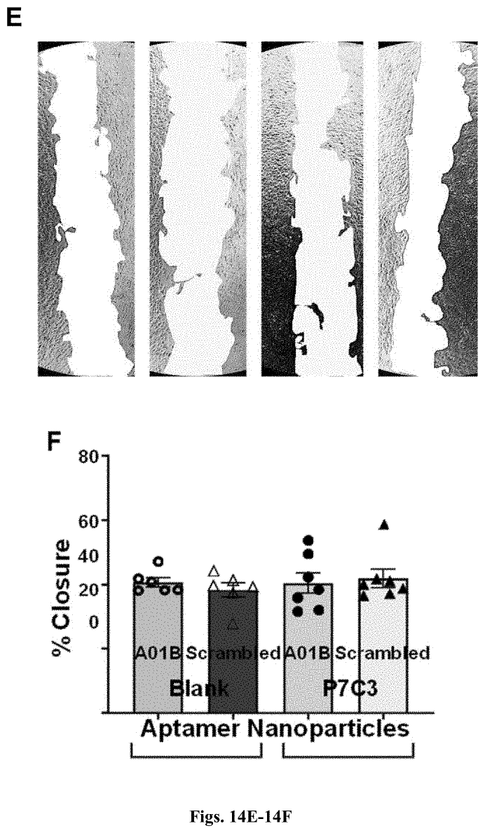

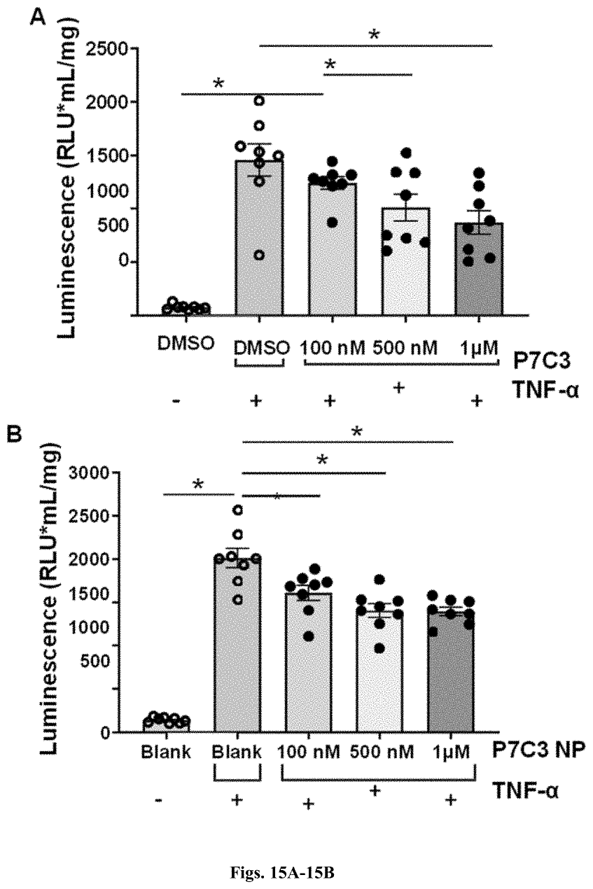

P7C3 attenuates inflammation in C2C12 cells. TNF-α (tumor necrosis factor α) triggers NF-κB, a master regulator of inflammation, so the C2C12 cells with NF-κB reporter cells were utilized as a model for the inflammation of C2C12 cells. TNF-α significantly increased the luciferase activity in the C2C12/NF-κB reporter cells (FIGS. 15A and 15B). 10 ng/ml TNF-α significantly increased the NF-κB activity compared with DMSO alone treated control cells. Both P7C3 and P7C3 NPs treated C2C12/NF-kB reporter cells showed a significant decrease in NF-κB activity with increasing concentrations, i.e., at 100 and 500 nM, and 1 μM concentrations compared with the DMSO+TNF-α inflamed cells. This data demonstrates that P7C3 and P7C3 NPs treatment of mouse C2C12 myoblasts for 24 hours decrease the inflammation induced by TNF-α.

P7C3 activates NAMPT, an enzyme that limits the reaction from nicotinamide to nicotine adenine dinucleotide (NAD), and activation of their pathway leads to increased cell growth, repair, and faster regeneration. NPs drug delivery is reported to enhance the cellular effects and is considered better for drug delivery and sustained release of the drug because they are encapsulated with PLGA polymers. The NPs delivery allows for enhanced permeation and retention of the compounds, allowing for a higher efficacy. PLGA NPs allows sustained release of P7C3 over time and is helpful in the continuous regeneration of cells. Aptamers are 3D structures made from single-stranded oligonucleotides that are 20 to 80 bases long and have a high specificity to certain target molecules; many aptamers have been developed to recognize cancer-associated antigens and can be useful in targeting the tumor. A01B RNA aptamer reported to bind with skeletal muscle cells and was retained following internalization. In this work, A01B RNA aptamer was used for muscle-specific targeting and conjugation with PLGA NPs loaded with P7C3.

Two key physical characterization parameters, particle size and surface charge determine the drug release profile, delivery of therapeutic agent, and outcome of the NPs. The particle size of developed NPs was in nano-range with average size of 182.9±6.3 nm for non-conjugated NPs and 255.9±4.3 nm for aptamer conjugated NPs (FIGS. 8A and 8C). The positive zeta potential of 10.4±1.8 mV showed surface charge on aptamer conjugated NPs, which will at the end prevent coagulation among the NPs by stabilizing them against electrostatic interaction forces.

The TEM images (FIGS. 8E and 8F) showed that the non-conjugated and aptamer-conjugated NPs were spherical with smooth surface and uniformly distributed, which was also in concordance with the low PDI using DLS technique. It is important to know about the in vitro drug release profile from polymeric NPs to correlate its usefulness in delivering therapeutic drugs with the in vivo effectiveness. As presented in FIG. 9, the P7C3 solution showed a 100% cumulative drug release within 24 h. This demonstrates that the solution form of drug can rapidly release the drug in the body, out of which only a fraction of drug can be absorbed or available for therapeutic effect and most of the drug can get metabolized. Furthermore, the shorter half-life of P7C3 will make it even difficult for an effective treatment option. However, there was a significant (p<0.05) change in the release pattern of the drug from non-conjugated as well as aptamer conjugated NPs compared to the P7C3 drug solution. Both the NPs exhibited sustained release pattern, which was due to the additional diffusional barrier presented by PLGA polymeric matrix. The NPs were also identified by RT-qPCR technique (FIGS. 10A-10C, and 11A-11C).

The cytotoxic study of cell viability with MTT assay showed that all three drug formulations, i.e., P7C3, P7C3 NP, and the P7C3 NP Apt were not toxic at time points analyzed except for a significant decrease in the cell viability with 1000 nM P7C3 NP, and 500 nM P7C3 NP Apt at 24 h. However, there was no significant difference in cell viability at 72 hours at higher concentrations such as 500 nM and 1000 nM for the three formulations of P7C3. Noticeably, there was a significant increase in the percent cell viability at 10 nM in both P7C3 and P7C3 NP groups at 72 h indicating an increase in proliferation and thereby increased mitochondrial activity converting MTT into formazan. This correlated with the wound healing at 16 h given the difference in seeding densities. Although a decrease in cell viability was noticed at 1 μM P7C3-NPs at 24 h time point the assay format for cell viability and scratch assay were different in terms of their seeding densities. Moreover, there was an increase in cell viability at higher concentrations, i.e., 500 nM and 1000 nM P7C3 NP Apt at 72 h but was found to be not significant (p=0.068 and 0.065, respectively). Together the data from MTT assay shows that all three formulations of P7C3 are not cytotoxic and increased cell proliferation at 72 h (FIG. 12).

Safety of PLGA polymer is key concern while making formulation and PLGA has demonstrated acceptable safety profile. PLGA has been widely used for preparing sustained release NPs and because of its biocompatible and biodegradable nature it has been approved by food and drug administration (FDA). Furthermore, P7C3 drug was entrapped in PLGA-COOH polymer matrix, which allowed sustained release of P7C3 from NPs to achieve an equilibrium between the quantity of drug and number of cells exposed to the released amount at a particular point of time thus maximizing the uptake and minimizing the undue drug overload to the cells. The A01B conjugated P7C3 PLGA-COOH NPs (P7C3 NP Apt) also showed similar cell viability. Overall, the MTT assay indicated there was no cytotoxicity of P7C3 NPs, i.e., with both the targeted and non-targeted P7C3s compared to P7C3 solution alone.

The time-dependent relative rise in the fluorescence of the NPs implies the cellular uptake of formulation in C2C12 cells. The relative MFI was significantly increased from 15 min to 60 min in the group of targeted NPs, which showed receptor-mediated cumulative uptake of the NPs in the cells with increasing function of time. However, no significant difference was found in relative MFI of scrambled conjugated NPs and it was found to be almost similar for 45 min and 60 min (FIGS. 13A and 13B).

In vitro injury model was utilized to test if P7C3 treatment resulted in increased wound closure in mouse C2C12 myoblasts. These results point to the idea that NAMPT activation allowed the wound to heal in a shorter time due to increased cell proliferation. NAMPT is expressed in different organs, so stimulating the NAMPT signaling pathway plays an important role in many biological mechanisms, such as energy metabolism and aging. Previously, studies have shown that NAMPT activation causes increased wound closure through mouse models that are mutated to have NAMPT knocked out or overexpressed. Because the wound closure of the myoblasts increases in a dose-dependent manner, the present data are in line with previous reports pointing that NAMPT activation is important for cell repair.

As described in the methods section, the in vitro model for generating wounds was performed by culturing C2C12 cells and treating them with the different agents of interest. The percent closure was calculated for the P7C3 solution, P7C3 NPs, P7C3 A01B aptamer conjugated P7C3 NPs, and scrambled aptamer conjugated P7C3 NPs after the 16 h time point. As shown in FIGS. 14A and 14B, percent wound closure increased with a higher dose concentration, which is statistically significantly when compared with the vehicle control group. FIGS. 14C and 14D conveyed that P7C3 NPs are more effective at lower doses compared to the P7C3 solution. When compared to the vehicle control group, the increase in wound closure was not significant for 100 nM of P7C3, whereas, it was significant for the 100 nM of P7C3 NPs solution (FIGS. 14C and 14D). The A01B conjugate PLGA NPs and scrambled conjugated P7C3 NPs did not demonstrate any significant increase in closure within the 16 h time mark (FIGS. 14E and 14F). The key reason for this is that the sustained release profile of NPs does not allow the release of P7C3 from targeted NPs to act on C2C12 cells within 16 h to promote wound closure.

NF-κB is the master switch, and is one of the major mediators of inflammation and is activated in muscular diseases; therefore, to mimic this pathway in vitro, C2C12 cells that have the NF-κB reporter via a firefly luciferase gene were utilized. Inflammation was triggered by TNF-α in the C2C12/NF-κB reporter cells. Previous studies utilized similar in vitro models for testing anti-inflammatory effects of steroids, such as prednisolone and celastrol, and showed decreased inflammation. Although the steroids do provide potent anti-inflammatory responses, glucocorticoids can bind the nuclear receptors quickly and produce its effects. Hence, there are major drawback for steroids as chronic use can lead to muscle and bone loss. Based on this fact, NAMPT was utilized as a new target to test if it has beneficial effects for skeletal muscle cells to overcome the side effects posed by glucocorticoids. Thus, in the present study, muscle inflammation at a fundamental pathway was evaluated and a NAMPT pathway was targeted to validate that NAMPT activation can promote anti-inflammatory responses in certain diseases, including muscle injury. The results obtained from this model point that P7C3, a NAMPT activator, significantly reduced the inflammation within the NF-κB signaling pathway, and it also showed that the nanoparticle version of this drug can reduce the inflammation at lower concentration. Additionally, nanoparticles are delivered in small, sustained releases over a long period of time, therefore the nanotechnology-based drug delivery allows for the extension of P7C3's effects. As a result, there is a potential for more effective mode of treatment than present modalities.

### CONCLUSION

A novel target-based approach for delivering P7C3 using biocompatible and biodegradable PLGA polymeric NPs was sought in the present work. Both A01B aptamer conjugated and no-conjugated P7C3 loaded PLGA-COOH NPs were designed and formulated successfully for effective delivery in skeletal muscles. The nano size and sustained release profile of designed NPs were found to be key characteristics for therapeutic delivery of P7C3 to the skeletal muscle with greater efficacy and safety. The A01B aptamer conjugated NPs showed higher cellular uptake in C2C12 cells over a period of one hour. Improved wound closure was noticed after 16 h of treatment in C2C12 cells when exposed to P7C3 solution and P7C3 NPs. The three formulations of P7C3 showed no cytotoxic effects on mouse C2C12 myoblasts cell viability. The luciferase activity was significantly reduced even in presence of TNF-α in the group of cells treated with P7C3 NPs after 24 h when compared to the P7C3 solution. The proposed A01B aptamer functionalized P7C3 PLGA NPs is useful for the effective treatment of skeletal muscle cell injuries and inflammation.

TABLE 1
Primers for A01B and Scrambled Aptamer

| | Sequence | Template strand of | | | | | |
|---|---|---|---|---|---|---|---|
| Name | (5′→3′) | A01B (72 bp) | Length | Start | Stop | Tm | GC% |
| A01B | Forward | GGACATATGATCAGGAGCCGAG | Plus | 22 | 11 | 32 | 60.09 |
| | primer | (SEQ ID NO: 1) | | | | | |
| | Reverse | AAGTGGTCATGTACTAGTCAAGCG | Minus | 24 | 82 | 59 | 60.62 |
| | primer | (SEQ ID NO: 2) | | | | | |
| Scrambled | Forward | GGACATATGATCGTCTCGATAGTG | Plus | 24 | 11 | 34 | 58.44 |
| A01B | primer | (SEQ ID NO: 3) | | | | | |
| | Reverse | CAAGTGGTCATGTACTAGTCAATCC | Minus | 25 | 83 | 59 | 59.19 |
| | primer | (SEQ ID NO: 4) | | | | | |

### Example 4. Nampt Activator P7C3 Ameliorates Diabetes and Improves Skeletal Muscle Function Modulating Cell Metabolism and Lipid Mediators

Type 2 diabetes mellitus is one of the most common and chronic metabolic disease worldwide. Type 2 diabetes has a debilitating impact on the whole body with major changes affecting the musculoskeletal system. It is mainly characterized by insulin resistance with the inability of the metabolic tissues such as skeletal muscle and liver to utilize insulin for maintaining glucose homeostasis, resulting in elevated blood glucose levels leading to hyperglycaemia. The occurrence of type 2 diabetes has increased rapidly in the last few decades and is the 7th major cause of mortality in the USA. There is increased prevalence of type 2 diabetes in younger population due to obesity, which is a predisposing factor to the metabolic disease. Due to an increase in the number of older populations, the incidence of type 2 diabetes has also increased worldwide. Type 2 diabetes is a major cause of cardiovascular diseases and neurodegenerative disorders. Diabetes affects skeletal muscle mass, strength and function through impaired energy metabolism, decreased blood flow, mitochondrial dysfunction and cell death.

Skeletal muscle is a major metabolic organ that not only utilizes glucose to produce the energy needed for contraction but also plays a key role in insulin stimulated glucose uptake in the postprandial state, and overall energy expenditure in mammals. Thus, skeletal muscle insulin resistance plays an important role in the pathophysiology of type 2 diabetes even before the pancreatic β cells dysfunction and the development of overt hyperglycaemia. Disruption of key insulin signalling pathways prevents the normal utilization of circulating blood glucose and storage of excess glucose as glycogen in the muscle and liver leading to hyperglycaemia and insulin resistance. Further, the secretion of pro-inflammatory cytokines by immune cells and the increase in lipolysis and free fatty acids, and the accumulation of intermediary lipid metabolites from other tissues such as adipose tissue can induce myocyte inflammation in obesity and negatively regulate the muscle cell metabolism resulting in insulin resistance. The skeletal muscle inflammation is also caused by the ectopic fat deposition, that is, inter-muscular and intramuscular lipid accumulation and can add to the degree of insulin resistance through paracrine effects. In addition, mitochondrial dysfunction has also been attributed in the development of insulin resistance and type 2 diabetes.

Nampt is a key rate-limiting enzyme in the NAD salvage pathway, which is dysregulated in the metabolic disease such as diabetes, and during ageing. Nampt is ubiquitously expressed, and the homozygous-null genotype is embryonically lethal in mice. Muscle-specific knockdown of Nampt in mice reduces the intramuscular NAD levels and causes myofibre degeneration and loss of physical functions like strength and endurance capacity mimicking type 2 diabetes in humans. Furthermore, knockdown of Nampt in mouse C2C12 myoblasts also reduces the NAD⁺ levels and results in decreased mitochondrial biogenesis. The Nampt activator, P7C3 was initially identified as a neuroprotective aminopropyl carbazole agent, which binds to and enhances the Nampt activity. However, the precise role of Nampt in skeletal muscle function and pathophysiology of metabolic disease such as type 2 diabetes is not known. In the present study, P7C3-mediated Nampt activation in the db/db mouse model of type 2 diabetes was shown to ameliorate the skeletal muscle diabetic phenotype and improve insulin sensitivity and function.

### Methods

Animals. The C57Bl/6J wild-type (WT), and the type 2 diabetic B6.BKS (D)-Lepr^{db}/J (db/db) male mice were purchased from Jackson laboratories. The Nampt heterozygous (Nampt^{+/}) mice were provided by Dr. Junichi Sadoshima (Rutgers Medical School, NJ, USA), and the colonies were bred and maintained at the Morsani College of Medicine, University of South Florida, USA. The 16-weeks old male db/db mice were treated with Nampt activator, P7C3 (db-P7C3) (10 mg/kg body weight for 4 weeks, daily, i/p). The vehicle control db/db (db-Veh) mice were treated with an equivalent dose of DMSO dissolved in 10% Kolliphor oil (Sigma-Aldrich, MO, USA) in phosphate buffered saline (PBS). The age-matched C57Bl/6J WT male mice were used as naïve control. In addition, 20-24 weeks old Nampt heterozygous (Nampt^{+/-}) and their littermate WT male mice were also treated with Nampt activator, 10 mg/kg body weight P7C3 (P7C3) once, i/p. The vehicle control Nampt^{+/-} (vehicle) male mice were treated with an equivalent dose of DMSO dissolved in 10% phosphate buffered saline (PBS), A total of 156 mice were used in this study, that is, 50 db-Veh and 51 db-P7C3 treated mice, and 35 C57Bl/6J WT and 10 each of the Nampt^{+/-} and littermate WT mice. All mice used in this study were fed with food and water ad libitum. All animal work was approved before start of the study by the Institutional Animal Care and Use Committee at the University of South Florida, Tampa, Fla., USA.

Fasting blood glucose and serum lipid. Mice were fasted 6 h, and tail vein blood was used for glucose measurement with ACCU-CHECK blood glucose meter (Roche Diagnostics, Mannheim, Germany) on a weekly basis for 4 weeks. At the end of 4 week treatment, blood was collected by retro orbital plexus technique for the assessment of serum lipids content using the Rat/Mouse Insulin ELISA kit (Miliipore, Mass., USA) following manufacturer's instructions. The serum high-density lipoprotein (HDL) and low-density lipoprotein/very low-density lipoprotein (LDL/VLDL) were quantified using HDL and LDL/VLDL quantification kit (Sigma-Aldrich, MO, USA US).

Insulin tolerance test. The glucose tolerance test (GTT) was performed in the Lepr^{-/-} (db/db) and Nampt^{+/-} mice. After 4 weeks of treatment, both the db-Veh and db-P7C3 treated mice were fasted overnight for GTT assay. GTT was performed by a single dose of intraperitoneal injection of 2 g/kg body weight of D-(+)-glucose (Sigma-Aldrich, MO, USA). Blood samples were obtained by the retro orbital plexus technique. Serum glucose levels were determined prior to glucose administration (0 min) and after 30, 60, 90, and 120 min of glucose injection, using glucose oxidase peroxidase kit (Pointe Scientific Inc., MI. USA) and following manufacturer's instructions. Similarly, the Namlpt^{+/-} and their littermate WIT mice were fasted overnight and given a single dose of vehicle control or P7C3 30 min prior to the administration of 2 g/kg body weight D-(+)-glucose, i/p. The circulating blood glucose levels were measured with ACCU-CHECK blood glucose meter at 0, 15, 30, 60, 120 minutes of glucose administration. The index of the total glucose shift was calculated as the area under the curve (AUC) using trapezium method.

Immunostaining. Immunostaining of the pancreas was performed for the measurement of insulin secreting β cells area, as previously described. Briefly, the frozen pancreatic tissues were fixed into tissue freezing media (TFM). Pancreas were cut into 10 μm thick sections and kept at −80° C. For immunostaining, the slides were first air dried and washed with phosphate buffer saline (PBS). The sections were then fixed in 4% paraformaldehyde for 30 min and blocked with SuperBlock™ T20 (TBS) blocking buffer (Thermo scientific, MA, USA) for 30 min, at room temperature (RT). Slides were then incubated with insulin primary antibody (sc-9168, Santa Cruz, TX, USA) diluted in blocking buffer (1:200), overnight at 4° C. Slides were then washed in PBS and incubated with Alexa Fluor®488 secondary antibody (Abcam, Mass., USA) diluted in blocking buffer for 1 h at RT. Following washes, the slides were then mounted with ProLong® Gold antifade mountant containing DAPI (Molecular Probe, OR, USA). Nuclei were labelled by DAPI. The images obtained with confocal multiphoton laser scanning microscope (Olympus FV1000 MPE, Olympus Inc., USA) were quantified by using ImageJ software (NIH, USA). The cross-sectional areas of pancreas and β cells were determined from multiple sections (n=3 mice per group), separated by at least 50 μm from each section. Pancreatic β cell area was calculated by using the following formula: islet β cell area (%)=(area stained by insulin anti-body/total islets area)×100.

Gomori aldehyde fuchsin staining. The Gomori aldehyde fuchsin staining of the pancreatic β cells was performed as per previously published protocol. Briefly, the frozen pancreas was TFM embedded and cut into 6 μm thick transverse serial cryosections using Microm cryostat machine. The slides were air dried at 60° C. and dewaxed using xylene and then hydrated through decreasing concentrations of ethyl alcohol. The slides were then rinsed in cold tap water followed by a single rinse in 50% ethanol and immersed in aldehyde fuchsin (Electron Microscopy Services, PA, USA) for 1 h. The excess stain was removed by rinsing in 70% ethanol and tap water. The slides were then counter-stained with Light Green (American Master Tech Scientific, CA, USA) through immersion for 60 s and dehydrated with increasing concentration of ethanol, and cleared with xylene. The sections were then mounted with disterene phthalate xylene (DPX) mountant (Sigma-Aldrich, MO, USA). Images were obtained at ×20 magnification with the Olympus IX73 inverted microscope using CellSens Standard software (Olympus Soft Imaging Solution, USA) and quantified with ImageJ software (NIH, USA).

Grip strength and voluntary running wheel. The fore-limb and hind-limb grip strengths were measured with the use of Chatillon force measurement DFE II grip strength meter (Ametek, Columbus Instruments, OH, USA). The average of five measurements per mice is represented as KGF/kg body weight for both the fore-limb and hind-limb grip strengths measured. The overnight voluntary running wheel experiments were carried out with the Columbus Instruments O₂₉₇, the CI Multi-Device Interface (Columbus Instruments, OH, USA) per manufacturer's recommendations.

Ex vivo myofibre contractility and force-frequency relationship. Animals were euthanized in compliance with University of South Florida IACUC guidelines. The extensor digitorum longus (EDL) muscles of each animal were tied with surgical silk at the proximal and distal tendons and dissected free. Muscles were immediately transferred to a customized vertical organ bath system (Aurora Scientific Inc., Canada) with the suture on the distal tendon clamped in place and that on the proximal tendon tied to a force transducer such that the muscles were positioned between two platinum wirestimulating electrodes. The buffer (137 mM NaCl, 0.4 nM NaH₂PO₄, 5.1 mM KCl, 1.05 mM MgCl₂, 2.0 mM CaCl₂) and 10 mM glucose; pH 7.4) in the bath was maintained at 22° C. and continuously aerated with 95%/5% O₂/CO₂. Muscles were equilibrated for 10-15 min after which, the optimal muscle length (l₀) for each muscle was calculated by adjusting the length until a maximal force response to a single twitch was attained. Following determination of l₀, the muscles were subjected to stimulation trains (500 ms duration) of frequencies ranging from 1-125 Hz, 1 per minute to determine the force-frequency relationship (FFR). Stimulation was delivered with a 58800 dual pulse digital stimulator (Grass Products, West Warwick, R.I., USA), and all pulses were 1 ms in duration. The peak forces of each contraction at the different stimulation frequencies were used to plot the FFR. Muscle force was expressed in both absolute (mN) and normalized terms (mN/mg muscle mass) of myofibre FFR. n=5-6 EDL muscles from three mice per group.

Haematoxylin and eosin staining. The tibialis anterior (TA) muscle was dissected from the mice, and embedded in optimal cutting temperature (OCT), frozen in isopentane chilled in liquid nitrogen and stored at 80° C. The 10 μm thick transverse serial cryosections were cut at the mid-belly region using Microm cryostat machine, and stained with haematoxylin and eosin for the analysis of myofibre cross-sectional area of the WT, db-Veh and db-P7C3 treated mice. Images were obtained at ×10 and ×20 magnifications with the Olympus IX73 inverted microscope and were quantified using CellSens Standard software (Olympus Soft Imaging Solution, USA).

Transmission electron microscopy. The EDL muscles were fixed with 2.5% glutaraldehyde in 0.1 M sodium phosphate buffer for 2 h at RT, and overnight at 4° C., followed by washing in buffer and fixation with 1% osmium tetroxide in buffer for 2 h at 4° C. After rinsing in buffer, the samples were then dehydrated in ethanol and acetone at RT and infiltrated in acetone:pure resin (Embed812) mix in vacuum with desiccant for 1 h and in pure resin for 4 h and embedded in the embedding mix. Polymerization of the embedding mix was carried out overnight at 70° C. in the embedding moulds. Semithin (1 μm) and ultrathin (70 nm) sections were cut using a Reichert Ultracut microtome machine. The sections were then stained with 2% uranyl acetate and contrasted with lead citrate as per standard protocol. Images were taken at ×15,000, ×30,000, and ×50,000 magnifications with a JOEL1400 transmission electron microscope for the analysis of myofibre diameter and the mitochondrial area.

Mitochondrial DNA (mtDNA) copy number. Total DNA was extracted from gastrocnemius (GAS) muscle using DNeasy Blood and Tissue kit (Qiagen, Md., USA) according to the manufacturer's protocol. The quantitative real-time PCR (qPCR) analysis of the mtDNA/nDNA was performed using iTaq Universal SYBR green supermix (Bio-Rad Laboratories, CA, USA). The mouse 16S was used as the mitochondrial gene and HK2 was used as the nuclear gene to obtain the mitochondrial copy number.

Succinate dehydrogenase staining. The 10 μm thick transverse cryosections of TA muscles were cut at the mid-belly region using Leica CM 1860 AgProtect cryostat machine. Briefly, the tissue sections were air-dried for 45 minutes, and incubated in 50 mM sodium succinate dibasic hexahydrate and 1.25 mM nitroblue tetrazolium in 0.1 M phosphate buffer (Sigma-Aldrich, MO, USA) for 30 min. The reaction was stopped washing in deionized water, and the sections were dehydrated in a series of graded ethanol and cleared using xylene. The sections were then mounted with DPX mountant, and the images were captured at ×20 magnification of the Olympus IX73 inverted microscope using CellSens Standard software (Olympus Soft Imaging Solution, USA) and the succinate dehydrogenase (SDH) positive myofibres were quantified with ImageJ software.

Quantitative real-time PCR. Total RNA was isolated from GAS muscle using the Exiqon miRCURY RNA Isolation kit (Exiqon, Mass., USA) according to the manufacturer's protocol. Complimentary DNA from total RNA was synthesized with iScript cDNA synthesis kit (Bio-Rad Laboratories, CA, USA) and quantitative real-time PCR (qPCR) analysis was performed on key transcriptome targets using iTaq Universal SYBR Green Supermix (Bio-Rad Laboratories, CA, USA). The cDNA synthesis and qPCR procedures were performed as described previously. The mouse HPRT gene was used as an internal control.

Luminex Magpix IP-10 (C—X—C motif chemokine ligand 10) immunoassay. The serum inflammatory markers were analysed using a mouse cytokine/chemokine antibody-immobilized premixed 32-plex Luminex magnetic beads based 96-Well plate immunoassay kit (Milliplex Map kit—MCYTMAG-70K-PX32, EMD Millipore Corporation, MA, USA) according to the manufacturer's instruction. Briefly, the serum samples were diluted at 1:2 in the assay buffer and measured in duplicates along with the standards and quality controls. The serum samples were incubated overnight at 4° C. with the magnetic beads. The wells were then washed with wash buffer and incubated with the detection antibodies for 1 h at RT, followed by a streptavidin-phycoerythrin incubation for 30 min at RT. After washing, the plate was run on MAGPIX with xPONENT software. The mean fluorescent intensity data was analysed using a five-parameter logistic curve-fitting Multi Analyst software for calculating the cytokine/chemokine concentrations of the samples (n=6 mice per group).

Whole transcriptome sequencing (RNA Seq). The RNA Seq of GAS muscle was carried out using Novogene Next Generation Sequencing platform NovaSeq 6000 PE150 (Novogene, Sacramento, Calif., USA). Total RNA was extracted from GAS muscle and the RNA quality and integrity were determined. RNA Seq library was then prepared and the cDNA library quality were determined before sequencing with Illumina PE150 platform. The data generated were analysed between groups (n=3 mice/group) for bioinformatics analysis.

Bioinformatic analysis. The Heatmap, Volcano Plot and Venn diagram were generated using R 4.1.1 software for differentially expressed genes. The gene network and pathways were analysed using DAVID v6.8 for GO enrichment analysis with Benjamini significant P<0.05 for both up-regulated and down-regulated genes. The Gene interaction network construction and visualization was performed using Cytoscape v3.8.2. The identified treatment responsive differentially expressed genes were created with the Search Tool for the Retrieval of Interacting Genes and proteins (STRING) database. Default settings of confidence score and maximum additional interactors were used for visualization. The Molecular Complex Deletion (MCODE) plugin for Cytoscape was used to identify the net-work modules. Densely connected regions/clusters in the co-expression network were identified with the following parameters; degree cut-off=2, haircut, k-core=2, node score cut off=0.2 and max. depth=100.

Myofibre staining. The 10 μm thick transverse serial cryosections of TA muscles were cut at the mid-belly region using Microm cryostat ma-chine. Briefly, the tissue sections were blocked with 0.2% bovine serum albumin (BSA) and 10% normal sheep serum (NSS) in PBS-Triton X100 (PBST) for 1 h at RT. All washes were carried out with PBS. Following washing, the sections were incubated in 5 μg/mL of mouse monoclonal primary antibody against myosin heavy chain type-1 (A4.840s DSHB, IA, USA) in 0.2% BSA and 5% NSS antibody dilution buffer in PBST overnight at 4° C. After washing, the sections were then fixed with 10% neutral buffered formalin for 5 min at RT, washed and incubated in 1:300 dilution of biotinylated sheep anti-mouse IgG (ab6807, Abcam, Mass., USA) and 1:1000 dilution of rabbit anti-laminin (L9393, Sigma-Aldrich, MO, USA) in the antibody dilution buffer for 1 h at RT. Following washes, the tissue sections were then incubated in 1:400 dilution of streptavidin AF488 conjugate and goat anti-rabbit AF594 (Invitrogen, CA, USA) and then washed and mounted with Prolong gold anti-fade mounting medium with DAPI (Invitrogen, CA, USA). Images were obtained at ×10 and ×20 magnifications with the Olympus IX73 inverted microscope using CellSens Standard software (Olympus Soft Imaging Solution, USA) and the MyHC1 positive myofibres were quantified with ImageJ software.

Lipidomics. The lipidomic profiling of GAS muscle was performed using a liquid chromatography-tandem mass spectrometry (LC-MS/MS) method as described previously. Briefly, the GAS muscle samples were collected, and flash frozen in liquid nitrogen. Around 50-100 mg of the muscle tissue was homogenized using a bead-beating method, then the obtained supernatant was further cleaned and concentrated by solid phase extraction. The extracted LMs samples were dried under vacuum and stored at 80° C. for future LC-MS/MS analysis. The dried extracts were reconstituted with 50 μL of methanol, and 10 μL was injected into LC-MS/MS to observe lipid profiles in GAS muscles. The LC-MS/MS system components were from Shimadzu Scientific Instruments Inc. (Columbia, Md., USA). All analyses and data processing were completed with the use of Shimadzu Lab Solutions V5.91 software (Shimadzu Scientific, Tokyo, Japan).

Nampt enzymatic activity. The Nampt enzymatic activity was measured from the GAS muscles of WT naïve control, db-Veh and db-P7C3 treated mice using the commercially available CycLex Nampt Colori-metric Assay Kit (MBL international, MA, USA) as per manufacturer's instructions. The OD values were obtained at 450 nm with the Synergy Hybrid H4 96-Well Plate Reader (Biotek, VT, USA) using Gen5 software after the sample incubation at 30° C., and the Nampt enzymatic activity was assessed.

Serum pyruvate enzymatic activity. Serum pyruvate levels were measured using the pyruvate assay kit (Sigma-Aldrich, MO, USA) as per manufacturer's protocol. Briefly, 5 μL of the serum samples were added to each well along with the pyruvate assay buffer, probe solution and the enzyme mix, and incubated for 30 min at RT. The absorbance was measured at 570 nm in the Synergy H4 Hybrid 96-Well Plate Reader (Biotek, VT, USA) using Gen5 software, and the concentration of the serum pyruvate levels were assessed.

Statistical analysis. Statistical analyses were performed with GraphPad Prism 9.0.2. Two-way ANOVA with Tukey's multiple comparison post-hoc test was utilized for all groups. An unpaired Student's t test was utilized when comparing only two groups. For the myofibre force-frequency data, two-way repeated-measures ANOVA (treatment × frequency), with frequency as the repeated factor was used to test for the main effect of treatment and the treatment × frequency interaction. Un-less otherwise specified each black dot or square, and the red triangle represent the data obtained from a mouse. Data are expressed as mean±SEM. The comparison-wise error rate, α, was set at 0.05 for statistical analysis of all data analysed using GraphPad Prism 9.0.2 software (La Jolla, Calif., USA), where asterisk (*) denotes significant difference from the post-hoc test.

### Results

P7C3 treatment alleviates the fasting blood glucose, insulin resistance and glucose intolerance in db/db mice.

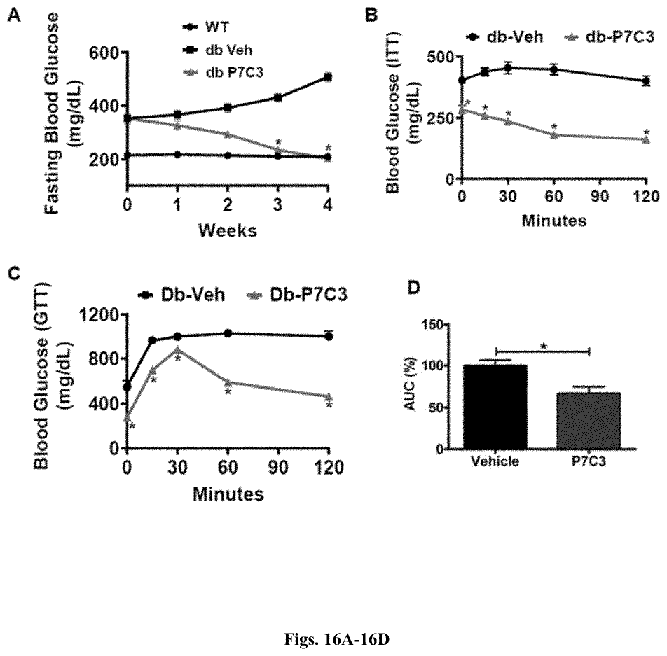

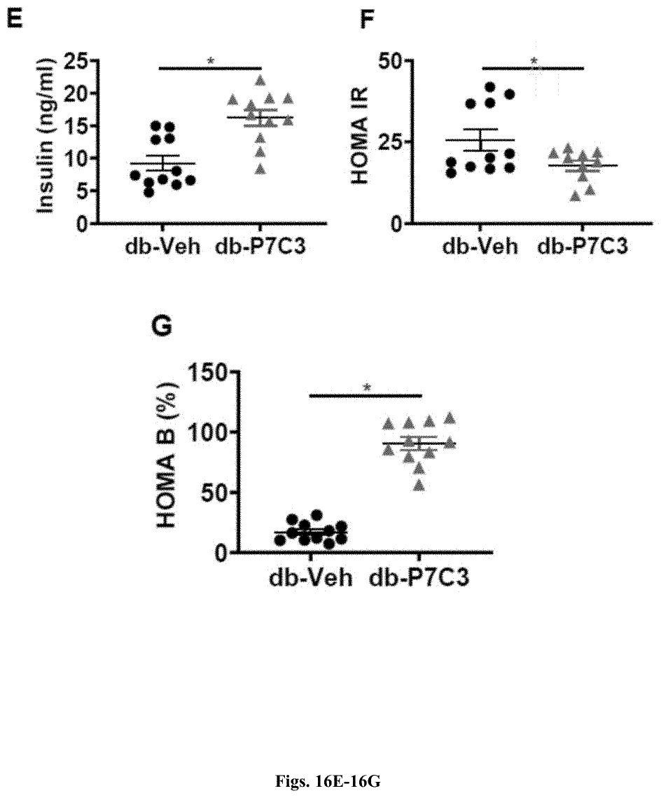

The db-P7C3 treated mice showed a significant decrease in the fasting blood glucose levels compared with the vehicle-treated db/db mice, while the db-Veh mice exhibited an increase compared with WT mice (FIG. 16A). The C57Bl/6J WT mice displayed the same amount of circulating blood glucose levels during the weekly assessment for the entire 4 weeks. These data indicate that P7C3 improves the insulin sensitivity or glucose utilization in the type 2 diabetic db/db mice. Circulating blood glucose levels in response to the ITT were found to be significantly reduced at all-time points analysed in db-P7C3 vs. db-Veh mice (FIG. 16B) confirming the insulin sensitivity of the db-P7C3 treated mice. A similar response to treatment was observed for the intraperitoneal GTT (FIG. 16C). The index of the total glucose shift calculated as percent AUC using trapezium method also displayed a significant decrease in the circulating blood glucose levels in the db-P7C3 mice compared with db-Veh mice (FIG. 16D). This validates that the db/db mice treated with P7C3 has increased glucose uptake, possibly through insulin-dependent mechanism. Subsequently, the circulating insulin levels were also increased in the db-P7C3 treated mice with decreased homeostatic model of insulin resistance (HOMA-IR) and increased β cells function (HOMA-B) compared with db-Veh mice (FIGS. 16E-16G). Together, this demonstrates that the intraperitoneal administration of P7C3 in the type 2 diabetic, db/db mice increases the glucose uptake, and promotes pancreatic β cells function.

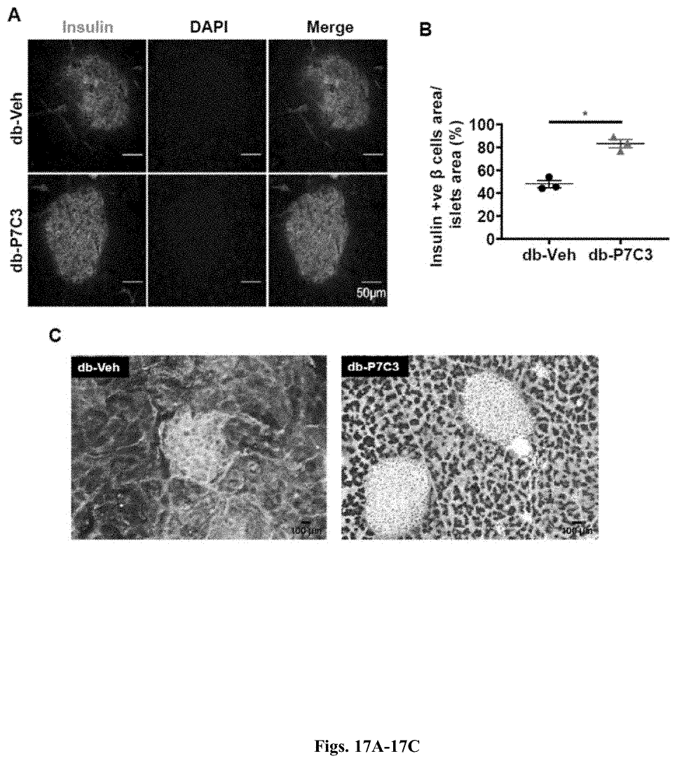

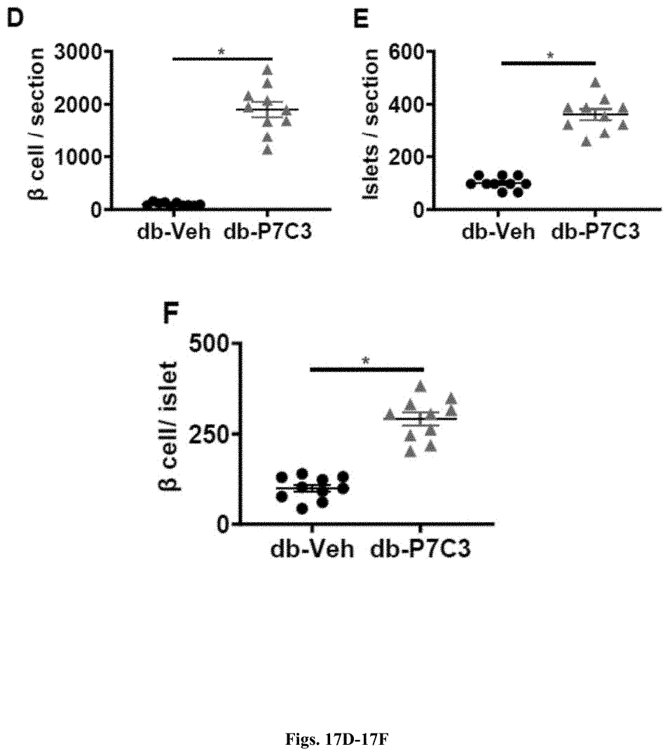

P7C3 treatment increases the number of pancreatic β cells in db/db mice. To assess whether db-P7C3 mice improve pancreatic β cells function of the islets of Langerhans, the db/db mice treated with vehicle or P7C3 for 4 weeks. Immunohistochemical analysis of the pancreatic islets of Langerhans displayed a significant increase in the insulin positive β cells area per islet of Langerhans area (FIGS. 17A, 17B). In addition, histological analysis of the pancreatic tissue cryosections stained with Gomori trichrome also displayed a significant increase in the number of β cells and islet of Langerhans per tissue section along with increased number of β cells per is-lets of Langerhans in the db-P7C3 mice compared with db-Veh treated mice (FIGS. 17C-17F). Taken together, this shows that there is increased number of pancreatic β cells in the P7C3 treated type 2 diabetic db/db mice, which contributed to the increased glucose uptake in the db-P7C3 mice.

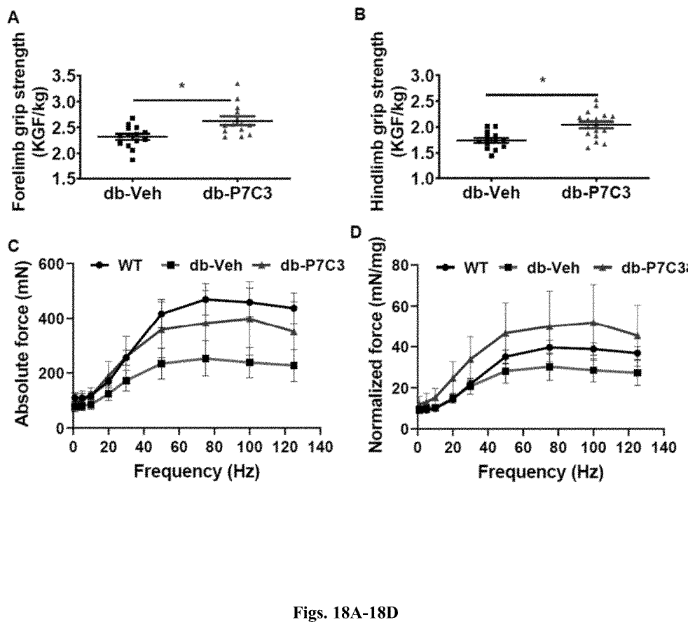

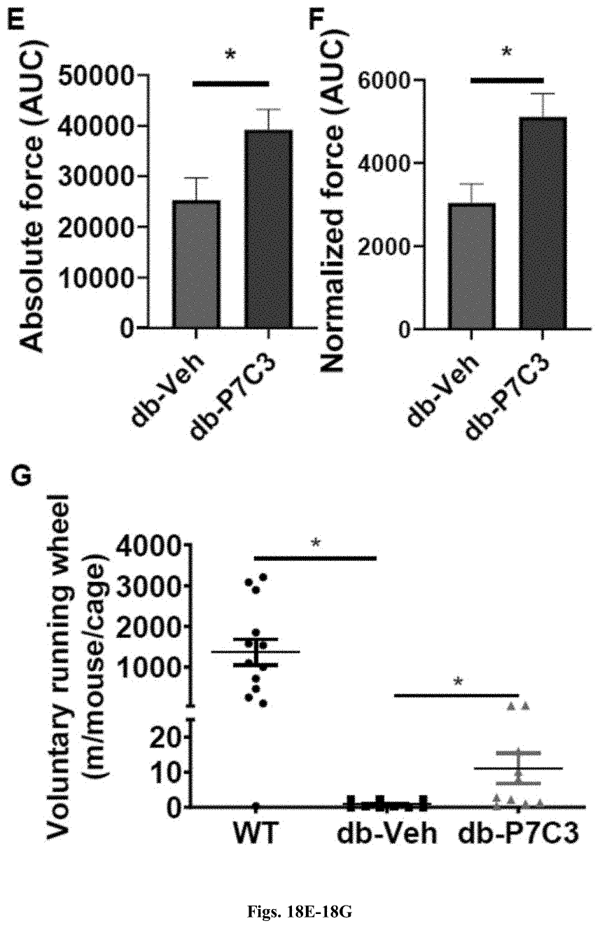

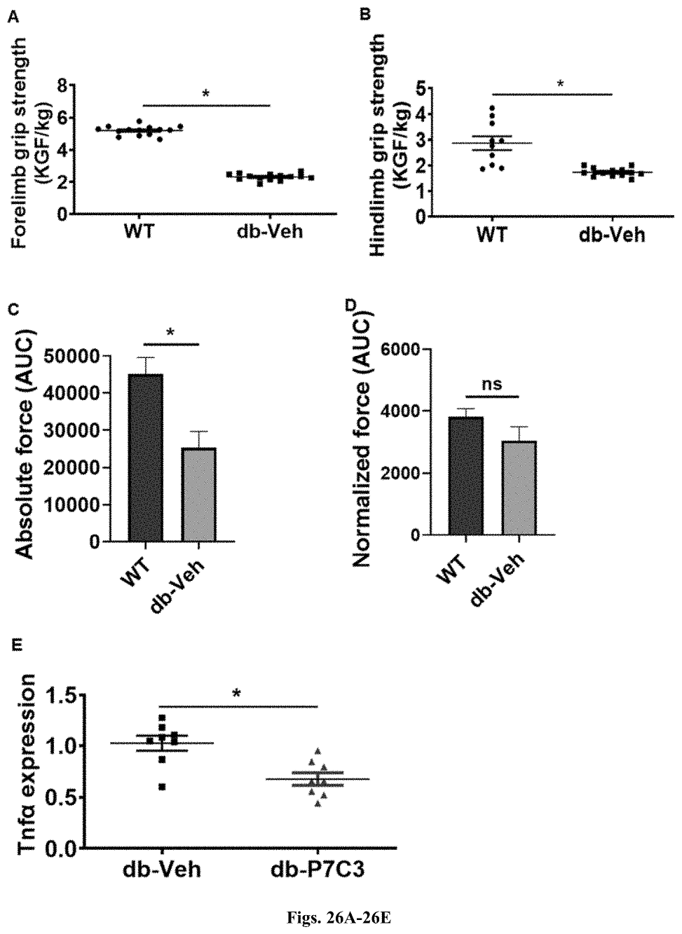

P7C3 treatment increases the physical performance of db/db mice. To determine whether P7C3 treatment modulates the physical performance in db/db mice, fore-limb and hind-limb grip strength and voluntary free running wheel performance, along with the ex vivo force frequency of the extensor digitorum longus muscle were compared. The db-P7C3 treated mice displayed a significant increase in the fore-limb and hind-limb grip strengths compared with db-Veh treated mice (FIGS. 18A and 18B). The db-Veh mice also displayed a significant decrease in the fore-limb and hind-limb grip strengths com-pared with WT mice (supporting information, FIGS. 26A,26B). There was significant group× frequency for both the absolute and normalized force frequency relationship data (P=0.05 and 0.04, respectively) when comparing the WT, db-Veh, and db-P7C3 mice, although no main effect of treatment was observed for either measure of the ex vivo extensor digitorum longus muscle contractility. These data indicate a trend for reduced force in the db-Veh group at higher frequencies, which is mitigated in the db-P7C3 group (FIGS. 18C, 18D). However, analysis of the area under curve through the trapezium method displayed a significant increase in the absolute myofibre force frequency in db-P7C3 mice compared with db-Veh treated mice (FIG. 18E). The db-Veh mice also displayed a significant de-crease in the absolute myofibre force frequency area under curve compared with WT mice (FIG. 26C). The normalized myofibre force frequency also showed a similar increase in the db-P7C3 treated mice (FIG. 18F). Even though the db-Veh mice displayed a decrease in the normalized myofibre force frequency area under curve compared with WT mice, it was found to be non-significant (FIG. 26D). In addition, the voluntary running wheel performance of the db-P7C3 treated mice also displayed a significant in-crease in the distance covered compared with db-Veh treated mice (FIG. 18G). Together, these data show that P7C3 treatment increases the physical performance of the type 2 diabetic db/db mice.

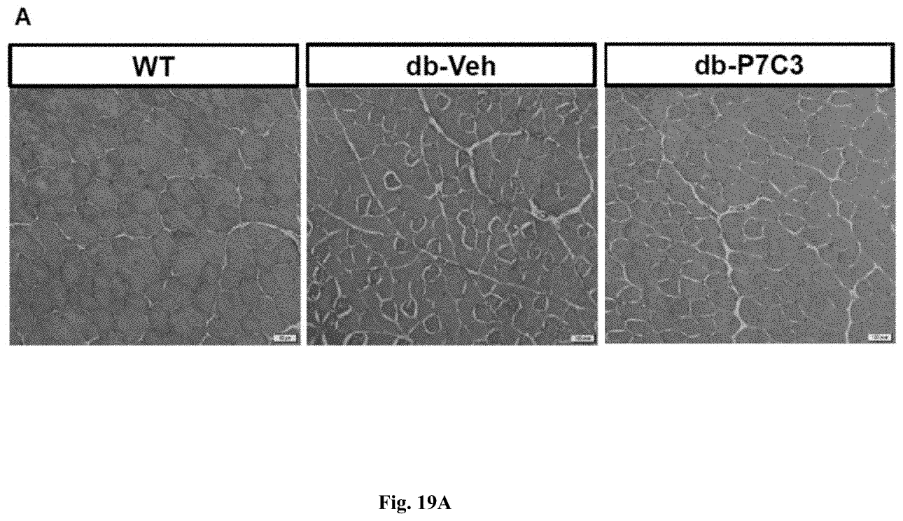

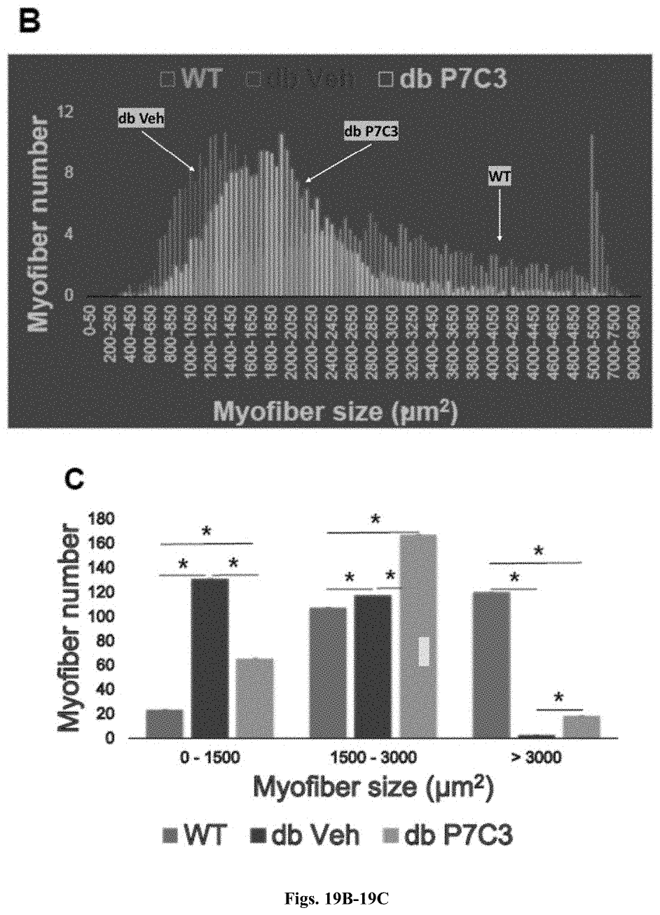

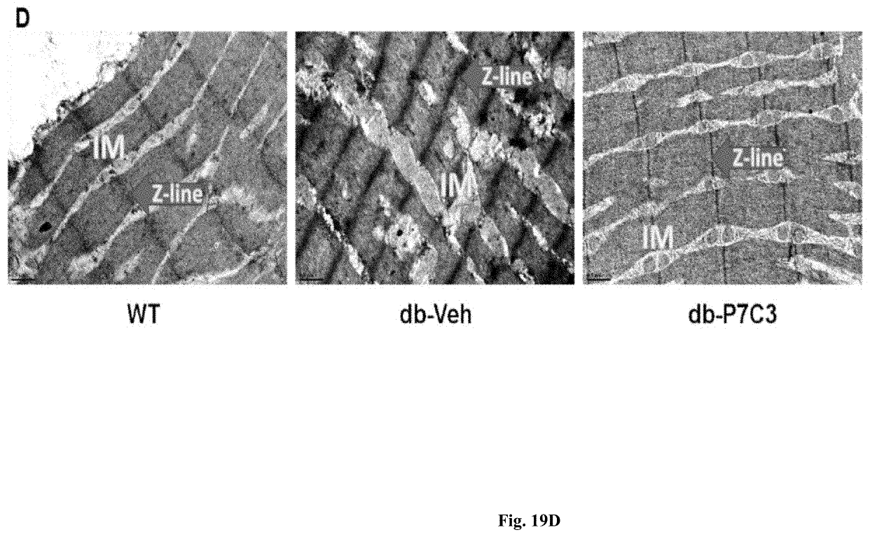

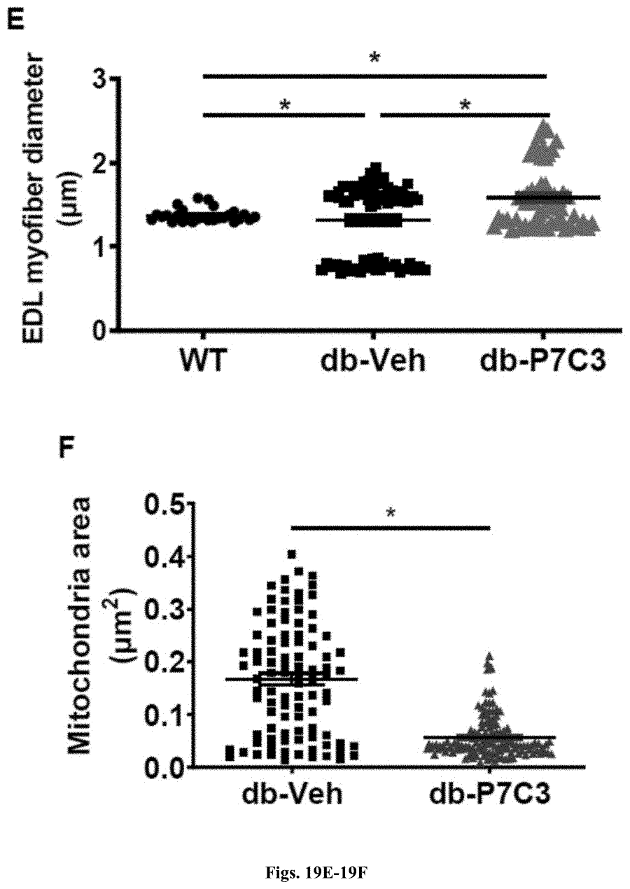

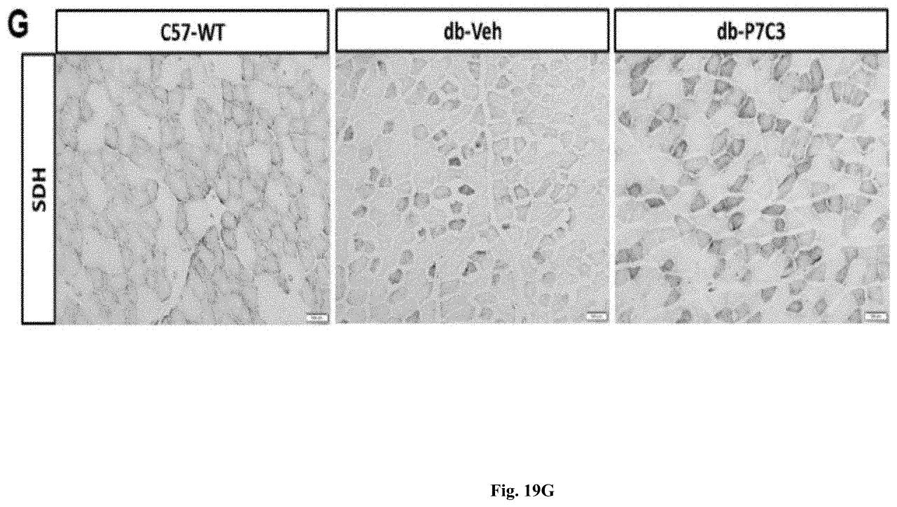

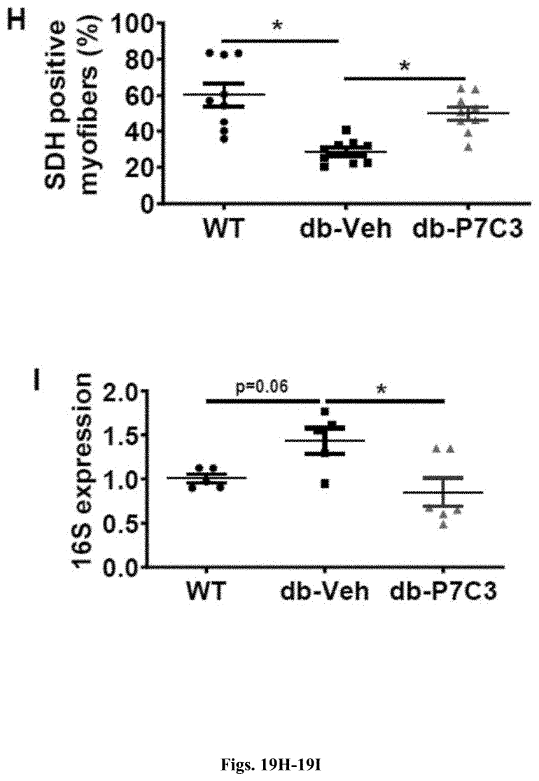

P7C3 treatment ameliorates the diabetic skeletal muscle phenotype of the db/db mice. The myofibre size of the type 2 diabetic db/db mice treated with vehicle or P7C3 were measured. Histological analysis of the haematoxylin and eosin (H&E) stained tibialis anterior muscle cross-sectional area around the mid-belly region displayed muscle atrophy with increased number of smaller (0-1500 μm²) myofibres cross-sectional areas in db-Veh treated mice compared with WT and db-P7C3 treated mice (FIGS. 19A-19C). The C57Bl/6J WT mice showed decreased number of small (0-1500 μm²) myofibres and increased number of medium (1500-3000 μm²) and larger (>3000 μm²) myofibres cross-sectional areas (FIG. 19C). Noticeably, the db-P7C3 treated mice displayed a significant decrease in the small myofibres cross-sectional areas compared with the db-Veh treated mice, with a shift towards an increase in medium and larger sized myofibres cross-sectional areas (FIG. 19A-19C). There was also a significant increase in the medium-sized myofibres and decrease in larger sized myofibres in the db-Veh treated mice compared with the WT mice (FIG. 19C). Subsequent transmission electron microscopy (TEM) image analysis of the longitudinal sections of the extensor digitorum longus muscles also displayed a significant decrease in the myofibre diameter in the db-Veh treated mice compared with the WT mice (FIG. 19D, 19E). Similar to the H&E-stained tibialis anterior muscle cryosections, the TEM images of the db-P7C3 treated mice also displayed an increase in the myofibre diameter of the extensor digitorum longus muscle compared with the db-Veh treated EDL muscle. However, it was found to be significantly lower than the WT mice (FIG. 19E). Interestingly, the TEM images also displayed in-creased mitochondrial area in the db-Veh treated mice compared with WT and db-P7C3 treated mice (FIGS. 19D, 19F). To assess the metabolic status and mitochondrial function in the skeletal muscle the SDH staining was performed. FIGS. 19G, 19H shows the decrease in the SDH activity in db-Veh mice compared with WT, whereas treatment with P7C3 for 4 weeks significantly improved the SDH positive myofibres in db-P7C3 mice skeletal muscle, To gain further insight into the mitochondrial DNA (mtDNA) content, 16S expression was measured. FIG. 19I shows the increased expression of mtDNA in db-Veh compared with WT (P=0.06), whereas, the 16S expression was significantly decreased in db-P7C3 group compared with db-Veh mice. Together, this shows that P7C3 treatment rescues the type 2 diabetic skeletal muscle phenotype of the db/db mice with an increase in the myofibre size and SDH positive myofibres, along with reduced mitochondrial copy number due to decreased stress resulting in improved mitochondrial structure and function.

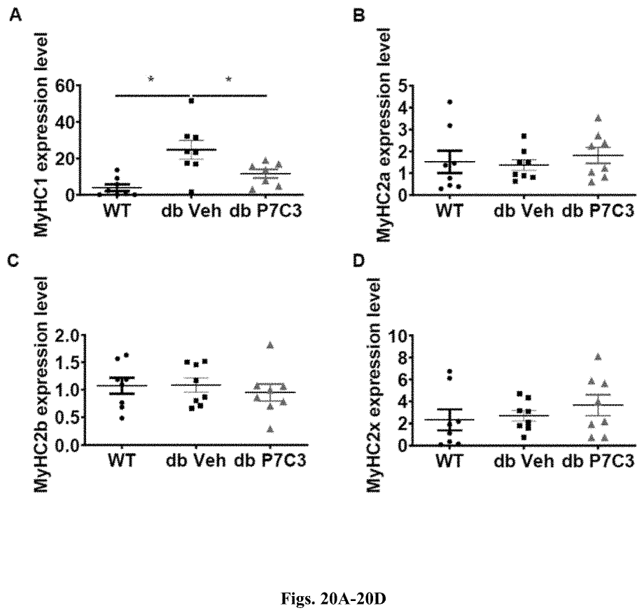

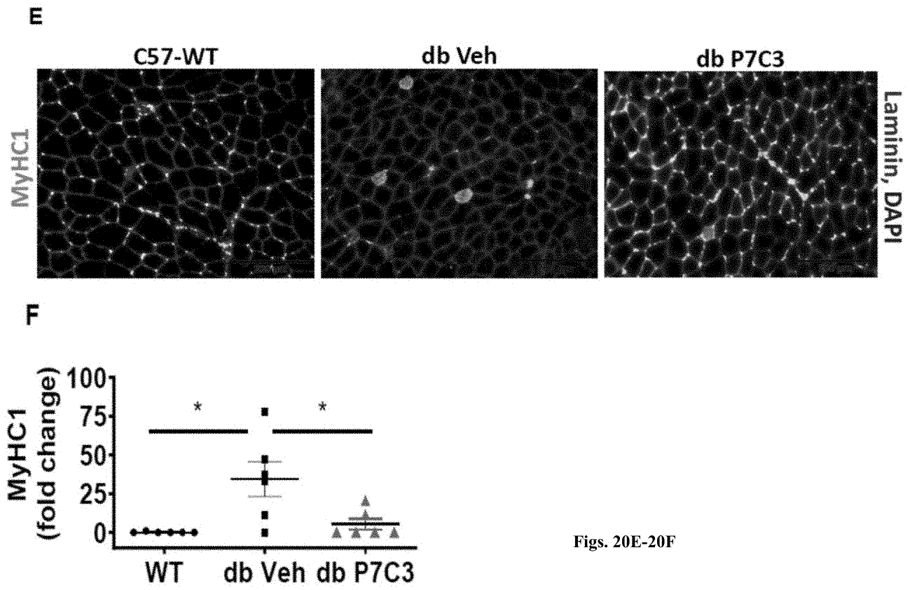

P7C3 treatment decreases MyHC1 expression and number in db/db mice. The myofibre types in the db-P7C3 treated mice were assessed next due to the observed shift in myofibre size. The db-P7C3 treated mice displayed a significant de-crease in the MyHC type 1 (MyHC1) expression levels in the gastrocnemius muscle compared with the db-Veh mice (FIG. 20A). Moreover the MyHC1 expression levels were found to be non-significant between the WT and db-P7C3 treated mice. A many-fold increase in the expression levels of MyHC1 in the db-Veh treated mice compared with WT mice was observed, showing an alternative increase of slow oxidative metabolism in the diabetic mice. Further, qPCR analysis of the db-P7C3 mice gastrocnemius muscle for myosin heavy chain fibre types, that is, MyHC-2a MyHC-2b, and MyHC-2× displayed no significant change in their expression levels compared with db-Veh treated mice and the WT mice (FIGS. 20B-20D). Subsequent analysis of the immunostained tibialis anterior muscle cryosections displayed a significant decrease in the number of MyHC1 positive myofibres in the db-P7C3 treated mice compared with db-Veh treated mice, which was found to be non-significant compared with the WT mice (FIGS. 20E, 20F). These data show that the diabetic skeletal muscle phenotype of db/db mice is rescued by P7C3 treatment with a decrease in the slow oxidative myofibres.

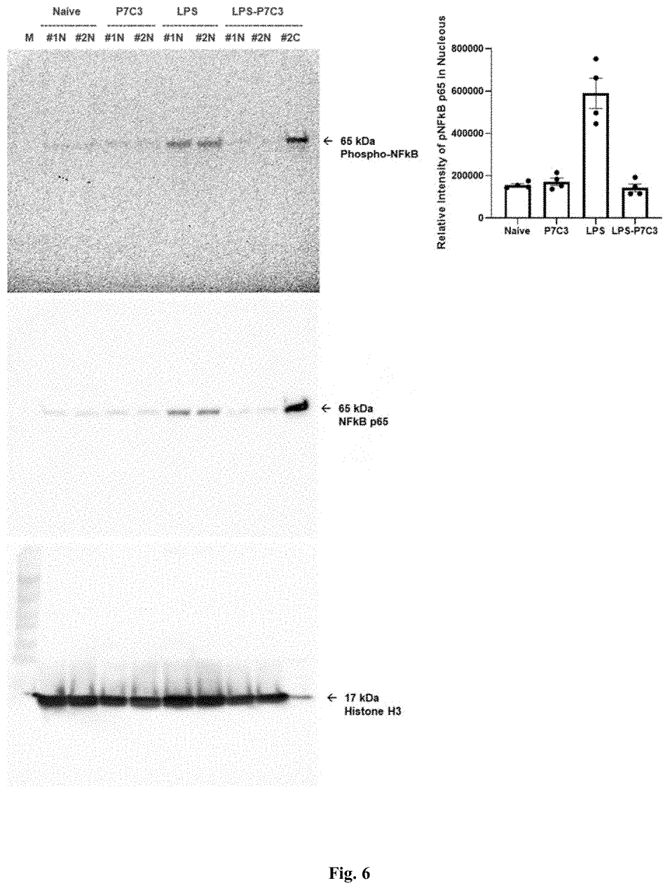

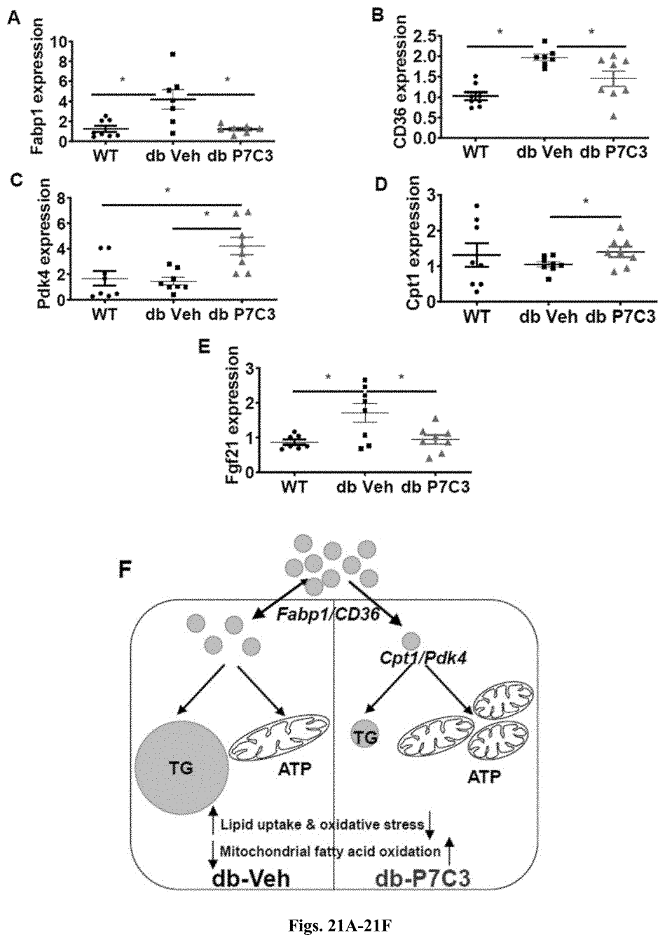

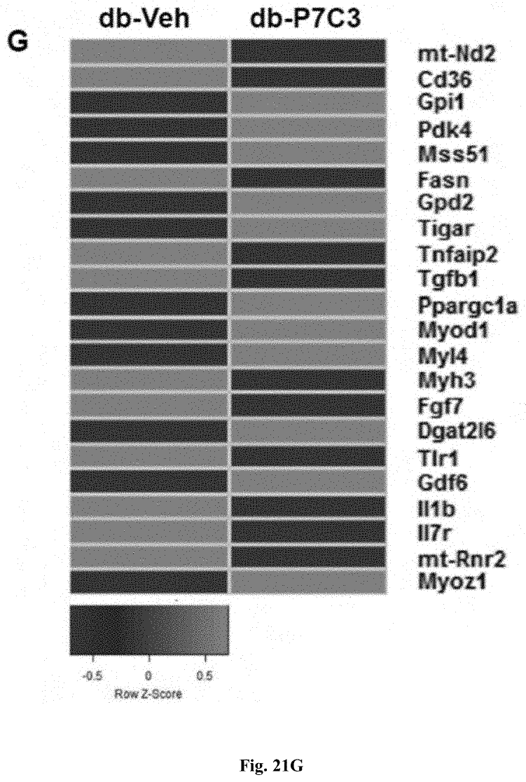

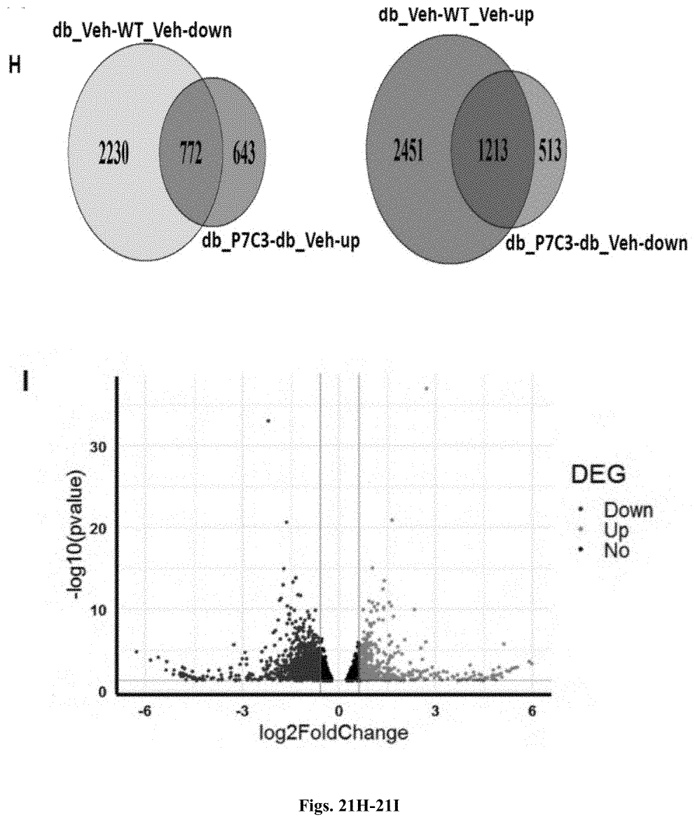

P7C3 treatment enhances mitochondrial fatty acid oxidation and decreases the myofibre stress in the db/db mice. Given that P7C3 treatment rescued the diabetic skeletal muscle phenotype of the db/db mice, a panel of metabolic markers were examined through qPCR gene expression analysis of the gastrocnemius muscle. The key genes involved in fatty acid uptake (Fabp1 and CD36) were found to be significantly down-regulated in the db-P7C3 treated mice compared with db-Veh treated mice, while there was no significant difference between the db-P7C3 and WT mice (FIGS. 21A, 21B). Whereas, the expression levels of key genes involved in fatty acid oxidation (Pdk4 and Cpt1) was found to significantly up-regulated in db-P7C3 treated mice com-pared to both db-Veh treated mice and the C57Bl/6J naïve control mice (FIGS. 21C, 21D). Noticeably, the db-P7C3 treated mice also resulted in a significant reduction in the expression level of Fgf21, the stress-induced myokine compared with the db-Veh treated mice, while it was non-significant compared with the WT mice (FIG. 21E). To evaluate the treatment responsive genes in diabetic skeletal muscle, RNA-seq analysis was performed. FIG. 6G shows heat map with significant key differentially expressed genes in the db-P7C3 group compared with db-Veh. The key genes include increased Ppargcla (PGC1a), decreased inflammation or stress responses along with improved myogenic genes with P7C3 treatment. As shown in the Venn diagram and Volcano plot in FIGS. 21H and 21I, respectively, the RNA-seq analysis showed high number of treatment responsive genes with 772/1415 genes up-regulated along with 1213/1726 genes down-regulated. Significant pathways were identified that are up-regulated due to P7C3 treatment, which include muscle structure development, metabolic pathways involved in energy metabolism (NADH, pyruvate, and ATP), calcium signalling, and glycolysis and gluconeogenesis. The pathways that are significantly down-regulated include inflammation (NFkB, IL-6,8, TNF, and cytokine production), TLR signalling, TGF-β, apoptotic and cell cycle processes, fatty acid elongation, and cytokine receptor and NADPH oxidase activity (FIG. 21J). Taken together, this shows that P7C3 treatment of the type 2 diabetic db/db mice increases mitochondrial fatty acid β-oxidation and thereby enhancing insulin sensitivity and energy metabolism of diabetic skeletal muscle phenotype with decreased oxidative stress and inflammation (FIG. 21F, 21G).

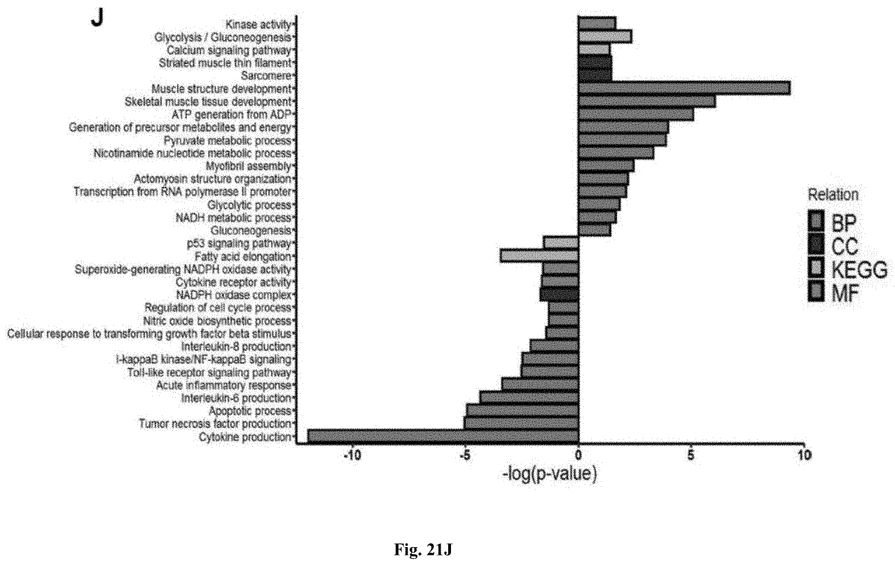

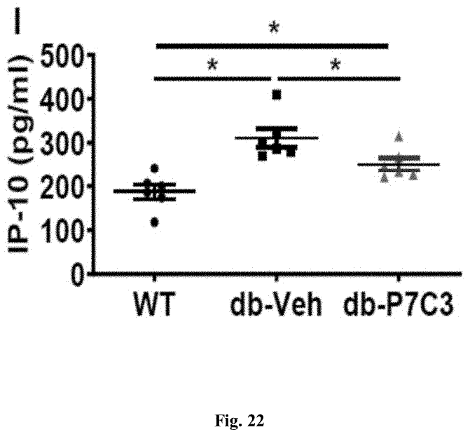

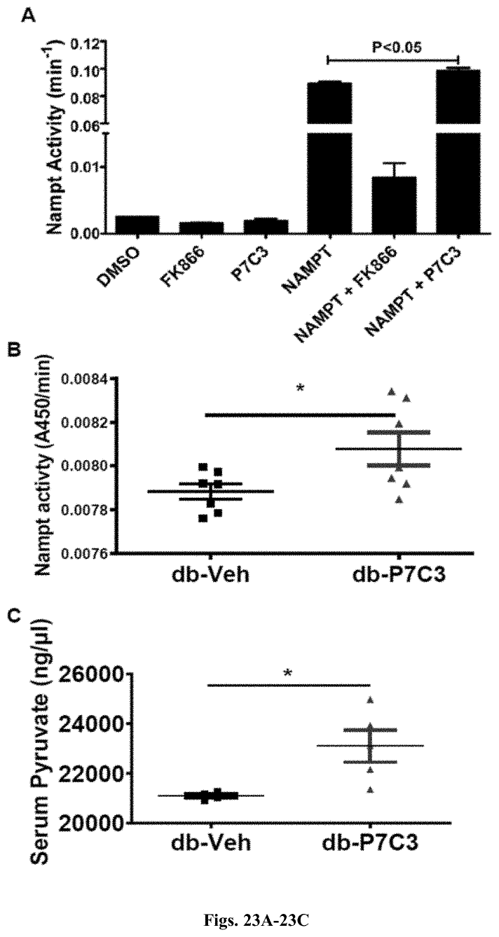

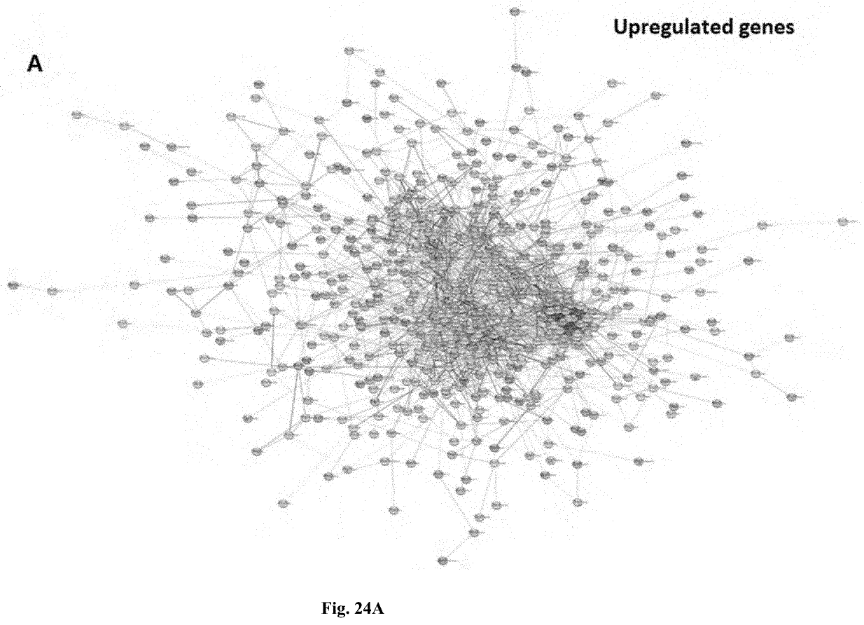

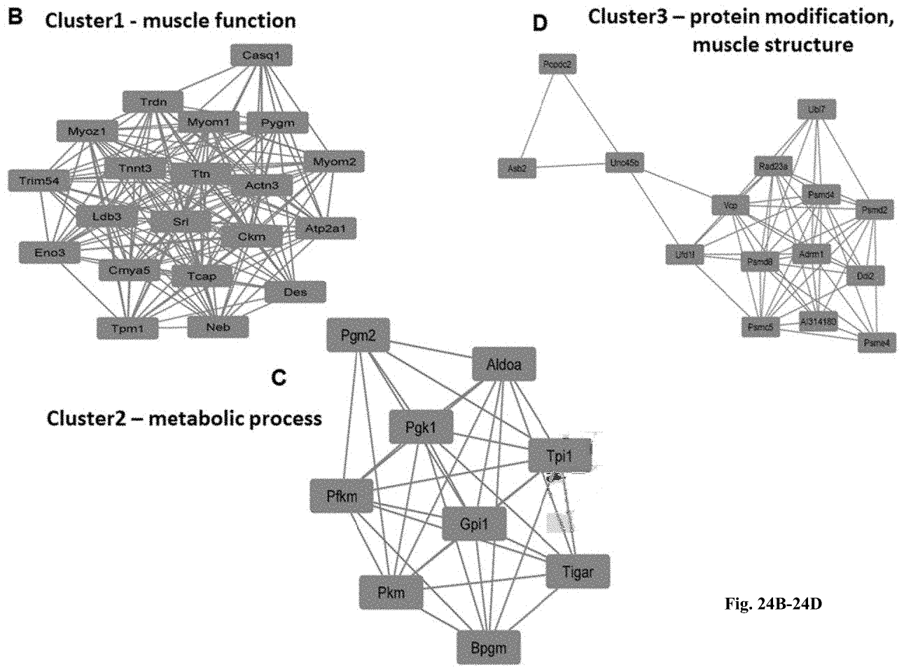

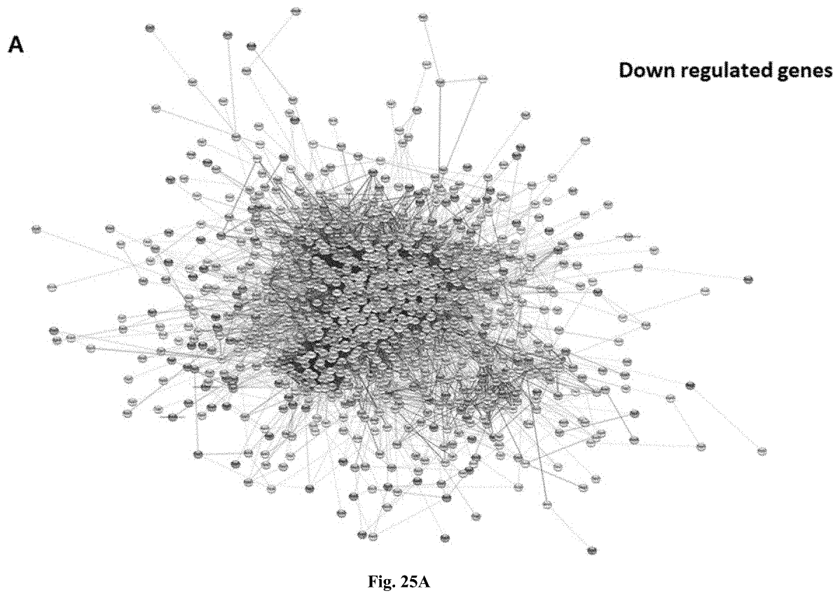

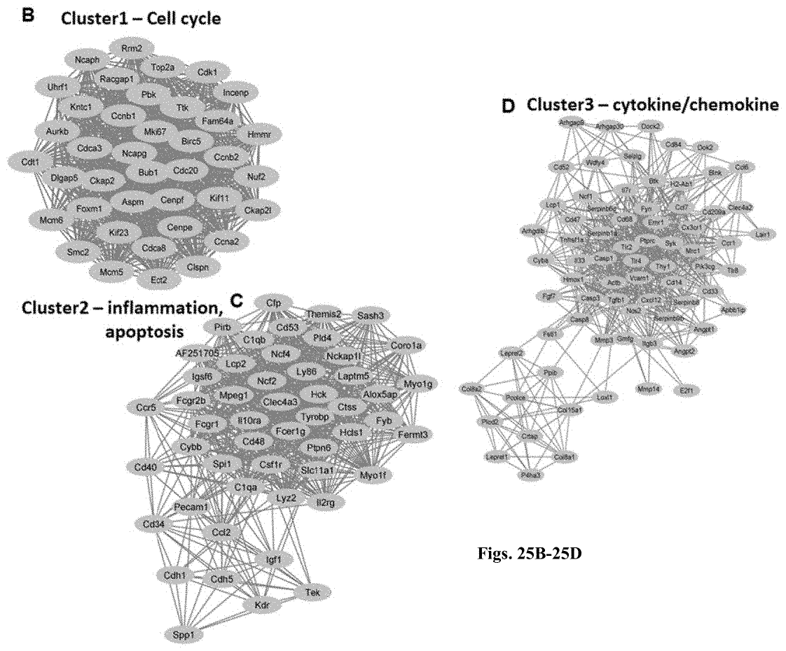

P7C3 treatment increases the circulating HDL levels and decreases the inflammatory lipid mediators of the db/db mice. The circulating levels of HDL and LDL/VLDL lipoproteins in blood plasma and the LC-MS/MS-based skeletal muscle lipid mediators were examined. The db-P7C3 treated mice displayed a significant increase in the HDL lipo-proteins level compared with the db-Veh treated mice. Furthermore, the db-P7C3 treated mice also displayed a significant decrease in the LDL/VLDL lipoproteins level compared with the db-Veh treated mice. Based on the increase in lipids in the obesogenic diabetic model, the lipids signalling in the skeletal muscle were further evaluated. The omega-6 polyunsaturated fatty acid (n−6 PUFA) arachidonic acid (AA) and its derivatives 11-HETE, 15-HETE, were significantly increased in db-Veh treated group, while P7C3 treatment of the db/db mice significantly attenuated the pro-inflammatory LMs in the gastrocnemius muscle. Additionally, P7C3 treatment of the diabetic mice also lowered the levels of the anti-inflammatory LMs, (n−3 PUFA) docosahexaenoic acid (DHA) and its derivatives, 7-HDoHE, 13-HDoHE, 16-HDoHE, 17-HDoHE, 20-HDoHE, and the eicosapentaenoic acid (EPA) and its derivative 5-HEPE and allowed the muscle to recover and bring the levels closer to that of the WT mice. The levels of endocannabinoid, 2-arachidonoyl glycerol (2-AG) was found to be significantly decreased in the db-P7C3 mice compared with the db-Veh treated mice, while it was more similar to the WT mice. However, anandamide (AEA) was found to be significantly higher in the db-P7C3 treated mice compared with the db-Veh and the WT mice. To verify the inflammatory cytokines/chemokines, interferon γ-induced protein 10 (IP-10/C—X—C motif chemokine ligand 10), a key pro-inflammatory target in serum was assessed using Luminex-Magpix magnetic bead immunoassay. As shown in FIG. 22, increased IP-10 levels were detected in db-Veh group compared with WT, whereas treatment of diabetic mice with P7C3 led to significant decrease in IP-10 levels compared with db-Veh, while it was still high compared with WT mice. To provide an in-depth analysis and overview of the gene net-work and pathways that are activated with P7C3 treatment in diabetic skeletal muscle the STRING network analysis was performed (FIGS. 24 and 25). The data show that the glycolytic, myogenesis, and NADH pathways were up-regulated, whereas the inflammation, apoptosis and stress responsive genes are down-regulated. Furthermore, TNFα expression was detected at high levels in db-Veh compared with db-P7C3 skeletal muscle (FIG. 26E). Overall, these data indicate that P7C3 treatment of the diabetic mice im-proves the circulating good lipoproteins and lipid mediators of the lipoxygenase (LOX) pathway (FIGS. 24-26) and contribute towards skeletal muscle recovery from the chronic low-grade inflammation associated with obesity and diabetes.

P7C3 treatment enhances the Nampt activity both in vitro and in vivo. Recent report shows that administration of P7C3 in animal models and tissue cultures enhance the enzymatic activity of Nampt, the key rate-limiting enzyme in the NAD salvage pathway from nicotinamide. Therefore, the P7C3 specificity of Nampt activity was tested using enzyme assay with or without P7C3 (activator), and FK866 (inhibitor) (FIG. 23A). As shown in FIG. 23A, the Nampt activity was found to be significantly decreased with the Nampt inhibitor (FK866) and increased with the Nampt activator (P7C3). The Nampt enzymatic activity was at its basal level and was found to be non-significant between the negative control DMSO, and the inhibitor and activator alone (FIG. 23A). Furthermore, db-P7C3 treated mice displayed a significant increase in the Nampt activity in gastrocnemius muscle com-pared with the db-Veh treated mice (FIG. 23B). In addition to Nampt activity, the levels of pyruvate in serum were assessed from diabetic mice treated with Nampt activator P7C3. As shown in FIG. 23C, the serum pyruvate levels were significantly high in db-P7C3 treated mice compared with db-Veh. Together, this shows the specificity of P7C3 as the Nampt activator, and these data also show that P7C3 administration increased Nampt activity in the type 2 diabetic db/db mice.

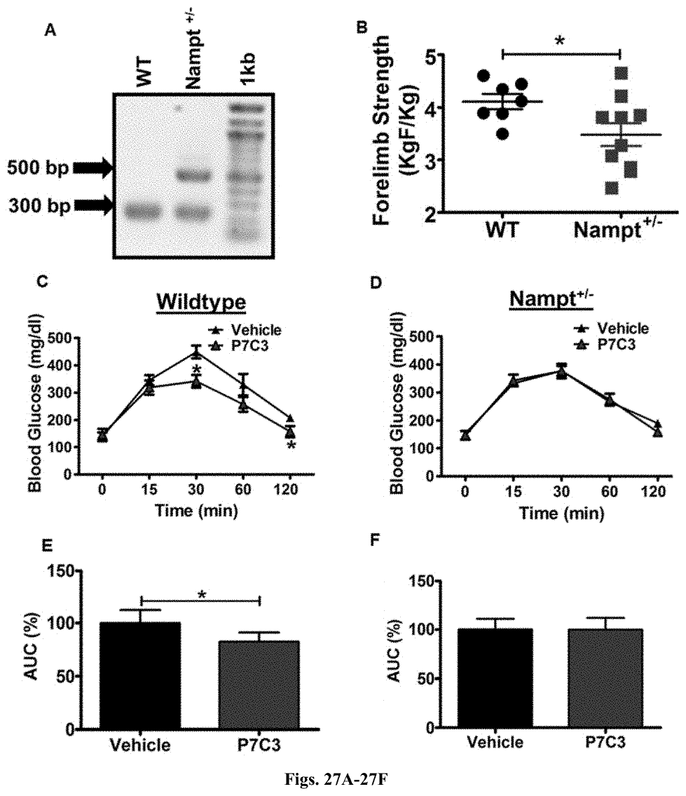

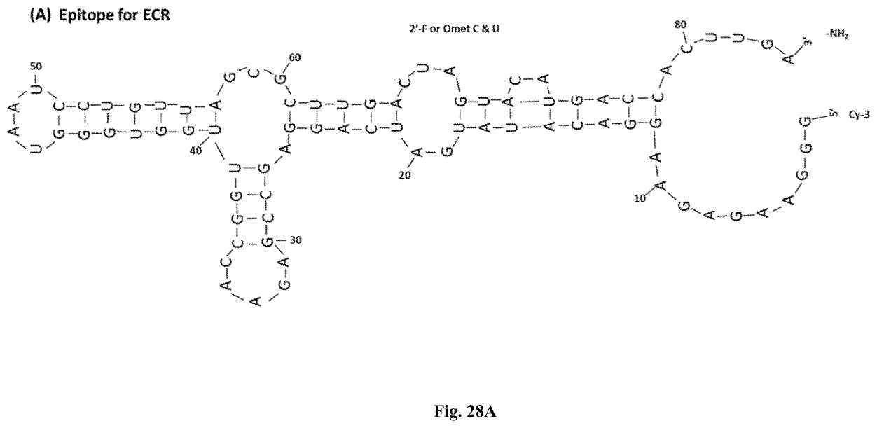

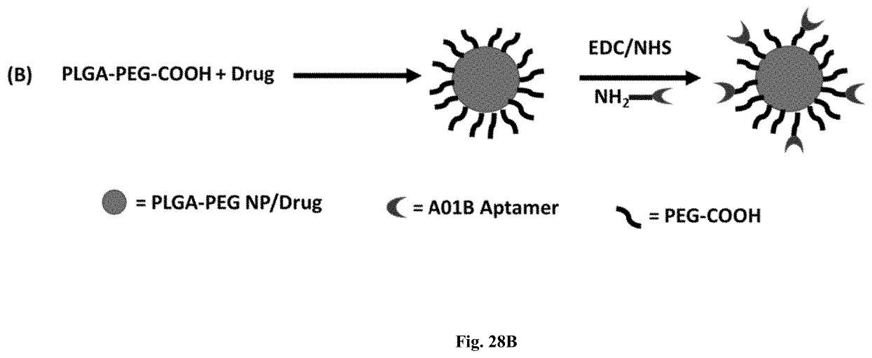

P7C3-mediated effects are Nampt-dependent. Next, to test whether the observed P7C3-mediated effects are Nampt-dependent, the Nampt heterozygous-null (Nampt^{+/-}) and their littermate WT mice were utilized and administered with a single dose of 10 mg/kg body weight P7C3, i/p. The tail snip PCR analysis clearly identified the Nampt^{+/-} and their littermate WT mice, with a double (300 bp and 500 bp) and a single (300 bp) intensity bands, respectively (FIG. 27A). The fore-limb grip strength of the Nampt^{+/-} mice was found to be significantly decreased compared with the littermate WT mice (FIG. 27B). Subsequent, GTT showed a significant decrease in the circulating blood glucose levels of the Nampt littermate WT mice treated with P7C3 compared with the vehicle-treated littermate WT mice. The Nampt^{+/-} mice treated with P7C3 did not show any significant effect on the blood glucose levels tested at all-time points analysed compared with the vehicle-treated Nampt^{+/-} mice (FIG. 27C. 27D). The index of the total glucose shift was calculated as per centAUC also displayed a significant decrease in the circulating blood glucose levels in the P7C3 treated Nampt littermate WT mice compared with vehicle-treated Nampt littermate WT mice (FIG. 27E), with no significant differences between the P7C3 and vehicle-treated Nampt^{+/-} mice (FIG. 27F). These data demonstrate that P7C3 modulates its anti-glycemic effect through the activation of Nampt and show that the amelioration of the hyperglycemic effect and its associated skeletal muscle phenotype observed in the type 2 diabetic db/db mice treated with P7C3 is Nampt-dependent.

The P7C3 is an aminopropyl carbazole that was reported as a neuroprotective compound in animal models of neurodegenerative diseases or nerve cell injury. Recently, it has been re-ported that P7C3-A20 treatment of C57Bl/6J mice on a high-fat diet rescued the fatty liver syndrome with improved insulin sensitivity and hepatic inflammation. P7C3 increases NAD synthesis from nicotinamide by activating Nampt, a key rate limiting enzyme in the NAD salvage pathway, and analogues of P7C3 have been shown to bind to Nampt. The enzymatic activity of Nampt and the bioavailability of NAD have been re-ported to be decreased in metabolic diseases and during ageing. Currently, a multidrug approach is aimed for the treatment of diabetes, which includes skeletal muscle and other tissues in the body. The P7C3-mediated activation of Nampt in the type 2 diabetic db/db mice ameliorates the diabetic skeletal muscle phenotype and improves the insulin sensitivity. In the present study, P7C3 was utilized as a novel compound in the amelioration of type 2 diabetic skeletal muscle phenotype and function in the Leprdb homozygous-null (db/db) mice. Furthermore, this data provides insights into the P7C3-mediated insulin sensitivity through Nampt activation.

Based on the study, the blood glucose levels of fasting blood glucose, insulin resistance, and GTTs were reduced in the db-P7C3 treated mice. Furthermore, there was increased number of pancreatic β cells and islets of Langerhans, and β cells function in the db-P7C3 treated mice. There was also increased physical performance in the db-P7C3 treated mice as measured with the fore-limb and hind-limb grip strengths, ex vivo myofibre force frequency and voluntary running wheel performance. Previous report identifies that doxorubicin induced decrease in NAD can be compensated by P7C3 via Nampt activity. This study also shows direct cross-linking of P7C3 with recombinant Nampt enzyme. However, previous studies did not report if the observed decrease in the blood glucose levels is through direct P7C3-mediated Nampt activation, involved in NAD biosynthetic salvage pathway or due to an indirect effect. To address this question, the recombinant Nampt enzyme was utilized and enzymatic activity assessed with P7C3 or the Nampt inhibitor FK866 in vitro. In addition, the type 2 diabetic, db/db mice treated with P7C3 or vehicle were utilized for assessing the gastrocnemius muscle Nampt and serum pyruvate activities. The Nampt enzymatic activity was observed to be increased with P7C3 and inhibited by FK866 in the in vitro assay, and it was also increased in the db-P7C3 treated mice. Similarly, the serum pyruvate levels were found to be increased in the db-P7C3 treated mice. To address whether the observed increase in insulin sensitivity and glucose uptake with the administration of P7C3 in the type 2 diabetic db/db mice is directly through Nampt activation, the Nampt+/− and their litter-mate WT mice treated once with P7C3 or vehicle were utilized for assessing the blood glucose levels of the intraperitoneal GTT. The glucose tolerance was improved in the Nampt litter-mate WT mice treated with P7C3 but did not improve in the Nampt+/− mice treated with P7C3. This clearly indicates that the P7C3-mediated effect on glucose uptake in the type 2 diabetic db/db mice is via Nampt activation.

In the db-P7C3 treated mice, there was an increase in myofibre size in hind-limb tibialis anterior and the extensor digitorum longus muscles, which shows a healthy muscle phenotype in the db-P7C3 treated mice. The type 2 diabetic db/db mice treated with P7C3 also displayed improved mitochondria morphology and function. The mitochondrial DNA was measured in the diabetic skeletal muscle and showed significant increase in 16 S expression, which might be due to increased oxidative stress and previous report have shown similar increase in diabetic muscle. Based on P7C3 treatment, 16 S expression was shown to be restored to the levels in WT muscle. More recently, P7C3 has been shown to stabilize the mitochondrial membrane potential in an in vitro dopaminergic cell culture model. In this study, P7C3 treatment of the type 2 diabetic, db/db mice resulted in the restoration of mitochondrial morphology and function. Furthermore, the observed shift in myofibre size from small to medium and larger myofibres was accompanied by a decrease in the MyHC1 (slow oxidative) myofibres expression levels and number, with no significant difference in the other MyHC fibre types expression levels in the db-P7C3 treated mice. Subsequently, a significant increase in the succinate dehydrogenase positive oxidative myofibres and increased Pgc1α expression in the db-P7C3 treated mice were both observed. Together this confirms that there is increased mitochondrial biogenesis in the P7C3 treated type 2 diabetic (db/db) mice that might be involved in energy metabolism. To test this, the expression level of the key markers in energy metabolism in gastrocnemius muscle examined and found decreased Fabp1 and CD36 expression levels along with an increase in Pdk4 and Cpt1 expression levels in the db-P7C3 treated mice. Strikingly, the expression level of the stress-induced myokine, Fgf21 was found to be decreased indicative of improved skeletal muscle health as evident in the H&E cryosections of the tibialis anterior muscle analysed for the myofibre transverse sectional areas and the TEM images of the longitudinal section of extensor digitorum longus muscle for the diameter. Together, this shows that there is decreased fatty acid uptake and increased mitochondrial fatty acid β-oxidation along with decrease myofibre stress and inflammatory markers, including IP-10 and Tnfaip2 in the type 2 diabetic db/db mice treated with P7C3 for energy metabolism and tissue homeostasis of the skeletal muscle.

Low HDL and increased VLDL levels in blood are often associated with insulin resistance and type 2 diabetes, which leads to increased risk of developing cardiovascular diseases. In this study, P7C3 treatment of the type 2 diabetic (db/db) mice showed a significant increase in the circulating levels of HDL along with a decrease in LDL/VLDL levels in blood. Furthermore, excess lipid accumulation in skeletal muscle causes muscle inflammation, and their levels have been reported to be increased in type 2 diabetic and obese individuals and animal models. In this study, an increase in the long chain poly-unsaturated fatty acids (LC-PUFA), especially the arachidonic acid (AA, 20:4; n−6), docosahexaenoic acid (DHA, 22:6; n−3) and eicosapentaenoic acid (EPA, 20:5; n−3) and their derivatives HETE, HDoHE and HEPE of the lipoxygenase (LOX) pathway were found through LC-MS/MS lipidomics of gastrocnemius muscle of the type 2 diabetic (db/db) mice. The db-P7C3 treated mice significantly decreased the levels of AA, DHA and EPA, and their derivatives in gastrocnemius muscle, showing decreased muscle inflammation in the db-P7C3 treated mice. The arachidonic acid is also known to serve as a precursor for eicosanoids and endocannabinoids, and the most studied endocannabinoids are 2-arachidonoyl glycerol (2-AG) and anandamide (AEA). P7C3 treatment of the type 2 diabetic (db/db) mice also resulted in a decrease in 2-AG levels consistent with the AA levels in these mice. However, there was an increase in the AEA levels in the db-P7C3 treated mice, the significance of which is not known and can be involved in the neuromuscular junction. The increase in HDL cholesterol in the db-P7C3 treated mice contributes to the cardioprotective effects with increased cholesterol efflux, and anti-oxidative and anti-inflammatory properties. Moreover, the decrease in LDL/VLDL cholesterol levels in the db-P7C3 treated mice also decreases atherosclerosis in heart disease, and alleviates insulin resistance and type 2 diabetes. Furthermore, the lipidomic analysis reveal that P7C3 treatment of the type 2 diabetic (db/db) mice attenuates the skeletal muscle inflammatory disease. In the present study, STRING network analysis was utilized and key genes and clusters responsible for muscle development, calcium signalling, glycolysis and gluconeogenesis were identified as up-regulated, whereas the inflammatory genes, cytokine production and apoptotic genes were down-regulated (FIGS. 24 and 25). In line with the present findings, a previous report shows similar increase in inflammatory markers using human myocytes from type 2 diabetic individuals with dysregulated myogenesis and gene network associations for development of diabetes. Overall, based on P7C3 treatment and in-depth RNA-Seq and network analysis the beneficial molecular responses that attenuate diabetes and improves muscle function were identified.

In summary, intraperitoneal administration of P7C3 in type 2 diabetic (db/db) mice, daily for 4 weeks improved the glucose uptake by decreasing the fasting blood glucose and insulin resistance, and increasing glucose tolerance, along with increased number of pancreatic β-cells, islet of Langerhans, and β cells function. The P7C3 treatment also improved the physical performance (fore-limb and hind-limb grip strengths, ex vivo myofibre force frequency and the voluntary free running wheel performance) alleviating the diabetic muscle phenotype of the db/db mice. Subsequent, histological analysis of the hind-limb tibialis anterior and extensor digitorum longus muscles displayed an increase in the myofibre size, with a simultaneous decrease in both MyHC1 expression levels and immunostaining of the slow oxidative myofibres in the db-P7C3 treated mice. The transmission electron microscopy also revealed an improved mitochondrial morphology in the db-P7C3 treated mice, with improved mitochondrial membrane potential. The increased SDH positive myofibres and Pgc-1α expression in the db-P7C3 treated mice demonstrate improved mitochondrial function. Mechanistically, there was increased mitochondrial fatty acid β-oxidation with decreased fatty acid uptake and oxidative stress in the db-P7C3 treated mice in energy metabolism. RNA Seq analysis also displayed an increase in fatty acid oxidation, mitochondrial biogenesis, muscle function with reduced inflammation. The LC-MS/MS lipidomic analysis of gastrocnemius muscle also displayed a decrease in the inflammatory lipids, along with an increase in the circulating HDL and decrease in the VLDL/LDL lipids in the db-P7C3 treated mice resulting in reduced muscle inflammation. The serum pro-inflammatory marker IP-10 levels also displayed a decrease in db-P7C3 treated mice. The specificity of P7C3 treatment was confirmed with an increase in the Nampt activity both in vitro and in vivo. The GTT of the P7C3 treated.

Nampt+/− mice further validated the hyperglycaemic effects of P7C3 as Nampt-mediated. Together, these findings highlight the importance of P7C3-mediated Nampt activation in ameliorating the diabetic skeletal muscle phenotype of db/db mice and Nampt activator P7C3 as a treatment strategy in type 2 diabetes.

STRING network analysis. The gene network analysis and interaction studies were performed using STRING database between the experimentally known gene products and in silico approaches resulting in a hub network of 674 upregulated genes and 1186 downregulated genes. Further, MCODE (Molecular Complex Deletion) plugin was used to identify top clusters of densely connected regions. The top three clusters of upregulated and downregulated genes were shown in supplemental FIGS. 1 and 2. Nodes represent gene/protein while edges represent interactions. The upregulated gene clusters i.e., Cluster1, Cluster2 and Cluster3 showed 20 nodes and 167 edges, 9 nodes and 34 edges, 15 nodes and 58 edges, respectively. Cluster1 involves genes playing roles in muscle contraction and development including calcium signaling, Cluster2 involves in metabolic processes (glycolysis and gluconeogenesis) and Cluster3 genes shows roles in protein modification and muscle structure development. The downregulated gene clusters consist of 39 nodes and 737 edges for Cluster1, 51 nodes and 744 edges for Cluster2, 73 nodes and 606 edges for Cluster3. Cluster1 is involved in nuclear division and cell cycle processes, Cluster2 includes inflammatory responses, TNF production as well as apoptotic processes, while Cluster3 genes are linked to roles in inflammation and cytokine-mediated signaling pathways.

### Example 5. Cardioprotective Effects of P7C3 in Diabetic Hearts Via Nampt Activation

Diabetes is associated with increased cardiac injury and sudden death. Nicotinamide phosphoribosyltransferase (Nampt) is an essential enzyme for the NAD⁺ salvage pathway and dysregulated in diabetes. Nampt activation results in rescued NADH/NAD⁺ ratios and provides pharmacological changes necessary for diabetic cardioprotection. Computer docking shows that P7C3 allows for enhanced Nampt dimerization and association. To test the pharmacological application, this study utilized male leptin receptor-deficient (db/db) mice and treated with Nampt activator P7C3 (1-(3,6-Dibromo-carbazol-9-yl)-3-phenylamino-propan−2-ol). The effects of four-week P7C3 treatment on cardiac function were evaluated along with molecular signaling changes for p-AKT, p-eNOS, and SiRT-1. The cardiac function evaluated by ECG and Echo were significantly improved after four-weeks of P7C3 treatment. Biochemically, higher NADH/NAD⁺ ratio in diabetic heart were rescued by P7C3 treatment. Moreover, activities of Nampt and Sirt1 were significantly increased in P7C3 treated diabetic hearts. P7C3 treatment significantly decreased the blood glucose in diabetic mice with 4-week treatment as noted by glucose tolerance test and fasting blood glucose measurements compared with vehicle treated mice. P7C3 activated Nampt enzymatic activity both in vitro and in the 4-week diabetic mouse hearts demonstrates the specificity of the small molecule. P7C3 treatment significantly enhanced the expression of cardioprotective signaling; p-AKT, p-eNOS, and Beclin 1 in diabetic hearts. Nampt activator P7C3 allows for decreased infarct size with decreased Troponin I and LDH release, which is beneficial to the heart. Overall, the present study shows that P7C3 activates Nampt and Sirt1 activity, decreases NADH/NAD⁺ ratio, resulting in improved biochemical signaling providing cardioprotection.

Cardiovascular disease (CVD) and diabetes mellitus (DM) remain major causes of mortality and morbidity in the United States. Primarily characterized as the occurrence of high blood sugar, diabetes is a progressive disease of hepatic and peripheral insulin resistance, β cells dysfunction and decreased insulin secretion. Diabetes increases the incidences of cardiac arrhythmias, myocardial infarction and sudden death. Cardiac electrophysiological abnormalities play an important role in diabetic cardiomyopathy, and ventricular arrhythmias are a major cause of diabetes associated death in patients. Electrically, QT prolongation is an increased risk factor for ventricular arrhythmias and sudden cardiac death leading to increased mortality in diabetic patients.

Although various genetic and environmental factors affect cardiac health and arrhythmia, emerging evidence indicates that pyridine nucleotides; nicotinamide adenine dinucleotide (NAD⁺) and reduced nicotinamide adenine dinucleotide (NADH), are major regulators of cardiac electrical activity. Nampt (Nicotinamide phosphoribosyltransferase) is an essential enzyme for intracellular NAD⁺ salvage, which is dysregulated in diabetes. Alteration of Nampt leading to a decrease in NAD⁺ and subsequent increase in NADH, can serve as a substrate for diabetic arrhythmogenesis, with potential to modulate electrical activity in the heart.

P7C3 was initially identified as a neuroprotective aminopropyl carbazole agent and tested in animal models of brain disorders. P7C3 binds to, and enhances, Nampt activity resulting in a significant increase in intracellular NAD⁺ levels, indicating that P7C3 stimulation of Nampt can be adapted to mitigate intracellular rises in NADH/NAD⁺ ratio in stressed cells of the diabetic heart. A recent report demonstrated that Nampt activation by P7C3 protects the diabetic skeletal muscle, however, the cardioprotective effects of P7C3 has not been investigated. This study tested whether Nampt activation by P7C3 rescues diabetic cardiac function. In the present study utilized P7C3 as a Nampt activator in db/db mice to show that P7C3 mitigates cardiac dysfunction by successfully enhancing key signaling molecules downstream of Nampt to promote cardioprotective features. The roles of NAD⁺ and Nampt were previously investigated as anti-ischemic and anti-aging factors, however, the effects in cardiac ischemia-reperfusion injury along with metabolic basis and signaling of Nampt activation by P7C3 remains unknown. The present study evaluated the activity of Nampt in diabetic hearts and identified pharmacological benefits that P7C3 offers in the diabetic heart via upregulating protective metabolism. Key signaling targets and links were studied to probe the status of a classic cardioprotective signaling mediators such as pAkt, and NAD⁺, which is Nampt dependent.

### Materials and Methods

Animals, research protocol approval and treatment. Male, B6.BKS(D)-Lepr^{db}/J (db/db) and C57Bl/6J (wildtype), mice were purchased from Jackson laboratory, stock number: 000697 and 000664, respectively (Bar Harbor, Me., USA) at the age of 10 weeks and housed with ad libitum food and water for 4-6 weeks and were used at ages 12-16 weeks. Nampt activator; P7C3 (1-(3,6-Dibromo-carbazol-9-yl)-3-phenylamino-propan−2-ol) was purchased from Cayman Chemical (Ann Arbor, Mich., USA). P7C3 was prepared by a previously reported method. P7C3 was dissolved in 2.5% dimethyl sulfoxide (DMSO; Sigma-Aldrich, MO, USA) in PBS containing 10% Kolliphor EL (Sigma-Aldrich, MO, USA). The db/db mice received daily intraperitoneal injections (i.p.) of P7C3 (10 mg/kg body weight) or an equivalent volume of the vehicle, for 4 weeks. A total of 14 vehicle control and 14 P7C3 treated db/db mice, aged 12-16 weeks, and 3 vehicle-treated and 3 P7C3 treated C57Bl/6J wildtype mice aged 16 weeks were utilized for this study. Additionally, a total of 8 C57Bl/6J wildtype vehicle controls were utilized as non-diabetic mice. Mouse hearts used for Western blotting, received insulin 5 minutes prior to tissue collection as discussed further in Western blot section. All experimental animal protocols were approved in advance by the Institutional Animal Care and Use Committee (IACUC) at the University of South Florida (Tampa, Fla., USA), and conform to National Institutes of Health (NIH) standards

Electrocardiography (ECG). Mouse ECG recordings were obtained utilizing 2-3% isoflurane/oxygen anesthesia in lead II configuration. ECGs were acquired for a total duration of 15 min, with 1 min recordings obtained at 5-minute intervals. Heart rate was monitored, while ECG traces were acquired using PowerLab system operated with LabChart 7.2 software (AD Instruments, UK). Data were analyzed offline using the ECG module of LabChart 7.2 software, as reported elsewhere. The intervals (ms) of QTc, JT, and ST elevation (mV) were measured. QT interval was measured from the start of the Q peak to the point where the T wave returns to the isoelectric baseline (TP baseline), and heart rate corrected QT (QTc) interval was obtained using the formula (QTc=QT/(RR/100)^{1/2}).

Echocardiography. Serial transaortic echocardiography was conducted under 2-3% isoflurane/oxygen anesthesia using a Visualsonic Vevo 770 system equipped with 30 MHz linear signal transducer (Toronto, Ontario, Canada). The mice were depilated as required for imaging and before placing on a 37° C. heated platform. Echo measurements were taken from at least three different cardiac cycles for each mouse. M-mode imaging from short-axis of the left ventricle (LV), using the papillary muscles for reference, was used to obtain measurements. Fractional shortening (FS %) and ejection fraction (EF %) were calculated as previously described.

Fasting blood glucose. Mice were fasted overnight, and the tail vein blood was used for measuring the blood glucose levels of the diabetic mice treated with either Vehicle (db/db Veh) or P7C3 (db/db P7C3) for 4 weeks. The blood glucose levels were measured using the ACCU-CHECK blood glucose meter (Roche Diagnostics, Mannheim, Germany).

Glucose tolerance test. The intraperitoneal glucose tolerance test (GTT) was performed in the overnight fasted db/db Veh and the db/db P7C3 treated (10 mg/kg body weight/day, i.p.) mice, and injected with 2 g/kg body weight of D-(+)-glucose (Sigma-Aldrich, MO, USA). Blood samples were obtained by the submandibular puncture technique. Serum glucose levels were assessed at 0, 15, 30, 60, and 120 minutes of glucose administration using the glucose oxidase peroxidase kit (Pointe Scientific Inc., MI, USA) according to the manufacturer's instructions. The index of total glucose shift between the treatment groups were calculated as the area under the curve (AUC) by using the trapezium method.

Ex vivo ischemia reperfusion protocol. Mice were injected with heparin (360 USP units, Sigma-Aldrich, MO, USA) and euthanized with Somnasol™ (pentobarbital, 50 mg/kg i.p. body weight). Hearts were then excised and mounted on to the Langendorff apparatus immediately and perfused with Krebs-Hanseleit buffer containing (in mM): 119 NaCl, 25 NaHCO₃, 4 KCl, 1 MgCl₂, 1.8 CaCl₂), 1 MgCl₂, 10 glucose and 2 Sodium pyruvate, pH 7.4, that was constantly bubbled with carbogen gas and maintained at 37° C. as described previously. Perfusion was maintained at a constant flow of ˜2.2 ml/min. Hearts were stabilized for 30 min before proceeding to the ischemic phase. Hearts that underwent P7C3 (3 μM) treatment during the Langendorff procedure were allowed to stabilize for 10 mins then a 20 min perfusion with P7C3 (3 μM) (within Krebs-Hanseleit buffer) as pre-ischemic dosing. Cardiac ischemia was induced by the pause in Krebs-Hanseleit buffer perfusion for 45 min. Reperfusion was induced with the induction of Krebs-Hanseleit buffer at a constant flow of ˜2.2 ml/min for 2 hours. Hearts that underwent P7C3 (3 μM) treatment received 2-hour reperfusion with P7C3 within the Krebs-Hanseleit buffer. Hearts were then removed from the Langendorff apparatus and immediately placed in −20° C. freezer for 30 minutes. Hearts were then placed on a sectioning block and sliced into (10 micron) slices. Heart sections were subjected to 2,3,5-Triphenyltetrazolium chloride (TTC) staining for 30 minutes. Hearts were then imaged and scanned for ischemic damage assessment. Image J software was utilized to quantify the ischemic areas for several sections per heart, which were then averaged and divided by the total area of the heart section and multiplied by a factor of ×100(%) to determine the overall damage percentage per heart.

Hearts undergoing LY294002 (Sigma L9908, 2-(4-Morpholinyl)-8-phenyl-1(4H)-benzopyran−4-one hydrochloride) (15 μM) exposure were performed with a modified ex vivo ischemia reperfusion protocol, used as PI3K inhibitor. The C57Bl/6J wildtype mice were injected with heparin (360 USP units, Sigma-Aldrich, MO, USA) and euthanized with Somnasol™ (pentobarbital, 50 mg/kg i.p. body weight). The hearts were then excised and mounted on to the Langendorff apparatus immediately, and perfused with Krebs-Hanseleit buffer containing (in mM): 119 NaCl, 25 NaHCO₃, 4 KCl, 1 MgCl₂, 1.8 CaCl₂), 1 MgCl₂, 10 glucose and 2 Sodium pyruvate, pH 7.4, that was constantly bubbled with carbogen gas and maintained at 37° C. Perfusion was maintained at a constant flow of ˜2.2 ml/min. Hearts were stabilized for 10 min before proceeding to pre-treatment phase. Control hearts were perfused with vehicle for 15 minutes followed by P7C3 (3 μM) for an additional 15 minutes. Hearts that underwent LY294002 treatment during the Langendorff procedure were allowed to stabilize for 10 mins then a 15 min perfusion with LY294002 (15 μM) followed by P7C3+LY294002 for an additional 15 minutes. Cardiac ischemia was induced by the pause in Krebs-Hanseleit buffer perfusion for 45 min. Reperfusion was induced with the induction of Krebs-Hanseleit buffer at a constant flow of ˜2.2 ml/min for 2 hours with P7C3 (3 μM). Hearts were then removed from the Langendorff apparatus and processed for Western blot assessment (see below).

Hearts undergoing ex vivo ischemia reperfusion protocol post-exposure with P7C3 (3 μM) were performed as defined above. Hearts were stabilized for 30 min before proceeding to the ischemic phase. Cardiac ischemia was induced by the pause in Krebs-Hanseleit buffer perfusion for 45 min. Reperfusion was induced with the induction of Krebs-Hanseleit buffer at a constant flow of ˜2.2 ml/min for 1 hour. Hearts that underwent P7C3 (3 μM) treatment received 1-hour reperfusion with P7C3 (3 μM) within the Krebs-Hanseleit buffer.

In vivo myocardial infarction (MI) protocol: The 16-weeks-old C57Bl/6J wildtype male mice were injected with a single bolus of 10 mg/kg body weight P7C3, i.p. or with an equivalent dose of vehicle control 30 minutes prior to the permanent ligation of the left anterior descending (LAD) coronary artery for generating the MI. In brief, mice were anaesthetized with CO₂, and intubated orotracheally and ventilated on a positive-pressure ventilator. All surgical procedures were carried out in aseptic conditions. A left thoracotomy incision was performed at the fourth intercostal space to expose the heart and ligating the LAD coronary artery with an 8-0 polypropylene suture to induce MI as described previously. Myocardial ischemia was confirmed with both the development of pallor myocardium distal to the ligation in the left ventricle and with the ST-segment elevation on the ECG. The skin and muscle incisions were closed with a 6-0 polypropylene suture, and the mice was administered a single dose long-acting analgesic Buprenorphine SR s.c. at 0.5-1.5 mg/kg body weight. The mice were monitored for any signs of pain or discomfort and its feeding and drinking behavior. After 24 hours of occlusion of the LAD coronary artery, the mice were euthanized as per the approved IACUC protocol, and the heart was excised for further processing with 2,3,5-Triphenyltetrazolium chloride (TTC) (Sigma-Aldrich, MO, USA). The whole heart was cut into six parallel short-axis sections and stained with 1% TTC for 90 minutes and fixed overnight in 10% neutral-buffered formalin (Sigma-Aldrich, MO, USA). The TTC stained heart sections were then scanned to determine the total infarct size as the ratio of the infarcted area, i.e., not stained with TTC to the ventricular area of the 4 sections from the apex to the site of occlusion (n=3 mice/group).

Pyridine nucleotide measurements. NADH/NAD⁺ ratios were determined in frozen db/db heart tissues (from the vehicle and P7C3 treated mice) using commercially available EnzyChrome kit (Bioassays, Hayward, Calif., USA) as reported before. Following manufacturer's instructions, cardiac tissue was homogenized in 100 μl of NAD⁺ or NADH extraction buffer for NAD⁺ and NADH determination, respectively. Samples were heated at 60° C. for 5 minutes on a heat block and neutralized by adding 100 μl of the opposite extraction buffer. Samples were then mixed by brief vortexing and centrifuged at 14,000 rpm for 5 minutes. 40 μl of supernatant from each sample was dispensed in duplicates in a 96-well plate, and 80 μl of working reagent constituted with supplier provided assay buffer, lactate dehydrogenase, lactate and MTT reagent was added to each well. Samples were gently mixed and optical density at 565 nm was acquired immediately (0 min) and after 15 minutes, at room temperature using a microplate reader. A standard curve was utilized for calculating NADH/NAD⁺ levels in each sample.

Nampt enzymatic activity. Nampt enzymatic activity was measured both in vitro and in vivo using the commercially available CycLex Nampt Colorimetric Assay Kit (MBL international, MA, USA). The db/db mice treated with vehicle and P7C3 for 4 weeks was utilized for harvesting the whole hearts. Briefly, the cardiac tissue was homogenized for protein extraction using T-per protein extraction buffer (Thermo Fisher Scientific, MA, USA). Following manufacturer's instructions, a 50 μg protein was used for Nampt activity measurement in each sample. OD values were obtained at 450 nm at 0 and 30 min after sample incubation at 30° C., and Nampt activity was measured.

Sirt1 deacetylase activity. Sirt1 deacetylase activity was measured in the whole heart lysates from the vehicle and P7C3 treated db/db mice according to the manufacturer's protocol using a deacetylase fluorometric assay kit (Sigma-Aldrich, MO, USA). The fluorescence intensity was measured at 440 nm (excitation, 340 nm).

Lactate Dehydrogenase activity. Briefly, coronary effluents were collected at varying time points (baseline, 30 minutes post-reperfusion, 60 minutes, and 120 minutes) and analyzed for LDH activity from vehicle and P7C3 treated wildtype hearts according to the manufacturer's protocol using LDH assay kit (Sigma-Aldrich, MO, USA). The absorbance intensity was measured at 450 nm.

Troponin I activity. Briefly, coronary effluents were collected at varying time points (baseline, 30 minutes post-reperfusion, 60 minutes, and 120 minutes) and analyzed for Troponin I activity from vehicle and P7C3 treated db/db and wildtype hearts according to the manufacturer's protocol using the Troponin I kit (Sigma-Aldrich, MO, USA).

Western blotting. For studies aimed to assess the insulin stimulated AKT phosphorylation, following four-week P7C3 treatment, mice were injected with Novolin R human insulin (1 Unit/kg body weight, i.p.), and hearts were collected after 5 minutes for Western blot analysis. Protein lysates for Western blotting were prepared from mouse hearts as described previously. Equivalent amounts of protein were loaded and resolved using 12% precast polyacrylamide gels (BioRad, CA, USA). Successful transfer of proteins was detected using Ponceau S (Sigma-Aldrich, MO, USA), and blots were probed with mouse monoclonal primary antibody for phospho AKT^{ser473} (p-AKT), total AKT (t-AKT), phospho eNOS^{ser1177} (p-eNOS), total eNOS (t-eNOS), Beclin1, and GAPDH (Cell signaling, MA, USA). HRP conjugated Rabbit anti-mouse antibody (Millipore) was used as a secondary antibody. Target protein band densities were quantified using Image J software.

Computational Modeling. Using the methodology described below, docking was performed using X-Ray crystal structures of Nampt obtained from the Protein Databank (www.rcsb.org). Although these structures consist primarily of co-crystalized complexes with inhibitors bound (and no activator complexes yet deposited) it has been demonstrated that activators and inhibitors share overlapping binding sites. Protein structure coordinates were obtained from the Protein Data Bank (PDB). Models were generated from PDB entry 4WQ6, the X-Ray structure used in a previously reported in silico study on NAMPT activators via computational docking. The Schrödinger Inc. software suite was used as the computational workflow for these studies. Protein model systems of Nampt were prepared using Schrödinger's Protein Preparation Wizard.

Protein Structure Refinement with Molecular Dynamics (MD). MD simulations were executed with the GPU accelerated Desmond MD program, on two Nvidia GeForce GTX 1080 Ti video cards. A cubic simulation box was generated and extended at least 10 Å from the protein with imposed periodic boundary conditions using TIP3P waters as solvent. The OPLS-3 all-atom force field was then applied to all atoms. Simulations were run at a temperature of 310 K and a constant pressure of 1 atm. All systems were energy minimized using multiple restrained minimizations to randomized systems before equilibration and final simulation. Production MD was subsequently performed on all systems for 250 ns.

Computational Docking. After MD equilibration was confirmed using a hierarchical average linkage clustering method, based on RMSD, an average representative structure for the equilibrated Nampt system was obtained. This representative Nampt structure was then used for docking the putative Nampt activator P7C3 using the Schrödinger software suite's GLIDE rigid receptor docking protocol with standard precision (SP) settings.

Statistical Analysis. Data are presented as mean±SEM. The unpaired two-sided Student's t test was used to compare the vehicle to the corresponding P7C3 treated groups. A one-way-ANOVA followed by Dunnett's post hoc test was conducted in NAD⁺ (%) when three groups were utilized to identify significant mean differences across the groups. A p≤0.05 value was considered statistically significant.

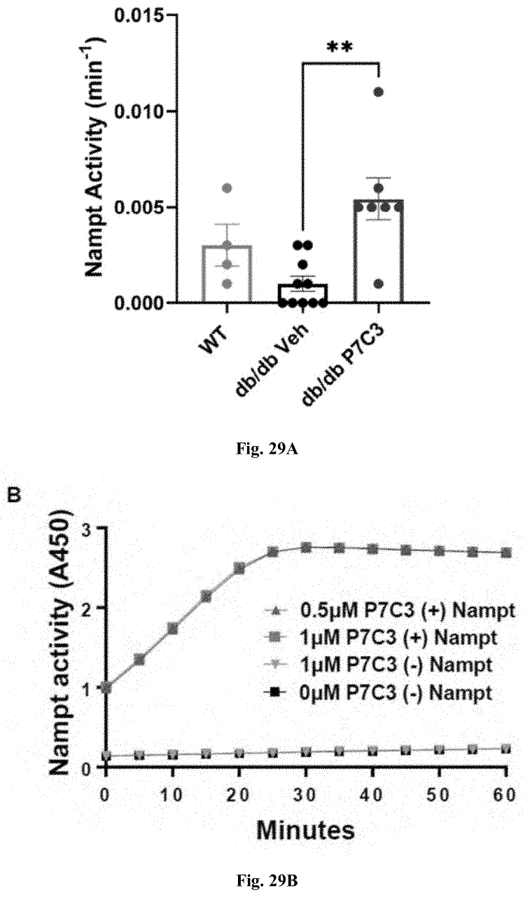

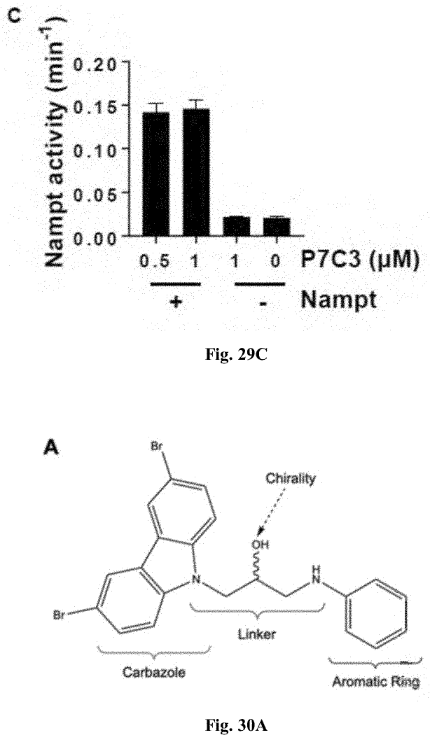

P7C3 enhanced Nampt activity in vitro and in vivo and improves NADH/NAD⁺ ratio. Nampt based NAD⁺ generation results in cardioprotection, however, the use of P7C3, a small molecule as Nampt activator in heart remains unknown. Early examination of the P7C3 small molecule has demonstrated the ability to bind and significantly enhance the enzymatic activity of Nampt. Diabetes remains a prominent risk factor for cardiovascular disease and likely plays a role in the deregulation of intracellular NAD⁺/NADH levels. Nampt remains a major regulator of the intracellular NAD⁺-SiRT1 axis and is a key regulator of pyridine nucleotide ratios. Therefore, we set out to examine the effect of P7C3 treatment on cardiovascular function within the diabetic mice model. A four-week treatment with P7C3 (10 mg/kg body weight/day, i.p.) significantly increased cardiac Nampt activity in db/db mice compared with vehicle treated mice (FIG. 29A). The observed increase of Nampt activity in db/db P7C3 treated mice was higher than wildtype control mice as well. We therefore sought to confirm the addition of P7C3 with an in vitro Nampt enzymatic activity assay, which resulted in enhanced activity (FIGS. 29B and 29C). Indeed, Nampt activity was increased with P7C3 at both 0.5 μM and 1 μM concentrations compared with the vehicle DMSO (0 μM P7C3), and 1 μM P7C3 without the recombinant Nampt enzyme (FIG. 29B). The Nampt activity was also measured by the absorption change per minute and was demonstrated as an increased overall activity with recombinant Nampt activity at 0.5 μM and 1 μM P7C3 concentrations (FIG. 29C).

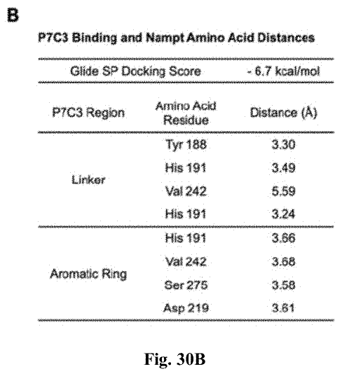

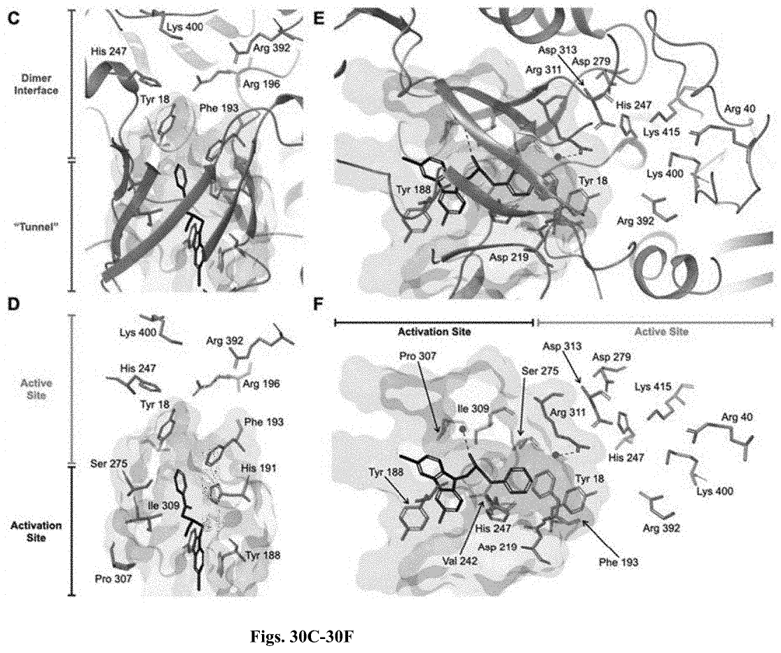

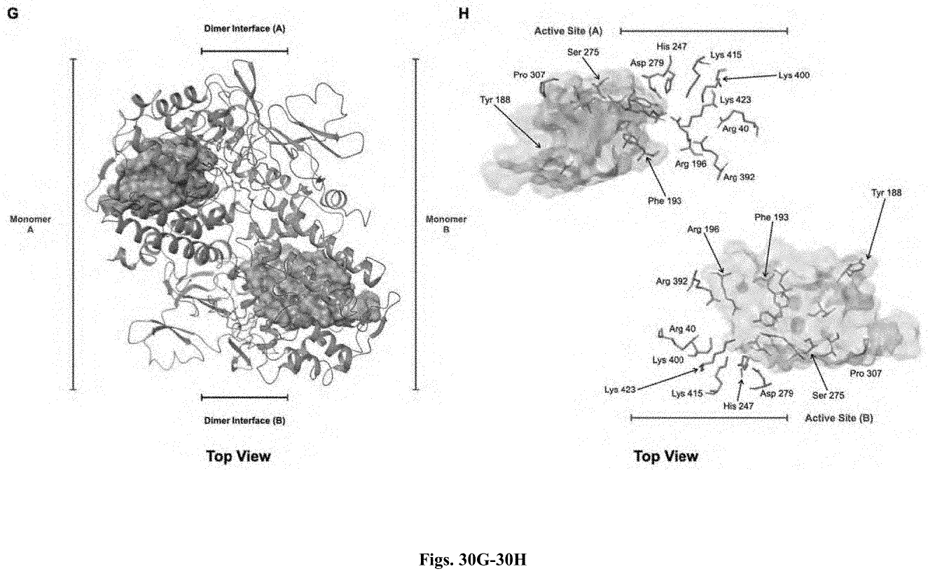

Nampt active site with P7C3 docking and visualization of interactions. Computational docking and visualization of the Nampt active site shows key amino acid residues in proximity to P7C3 allowing for tight interaction. FIG. 30A shows the carbon structure of P7C3 molecule with a carbazole and aromatic ring connected by a linker with chirality. As shown in FIG. 2B-H, five key amino acid residues, including tyrosine, histidine, valine, serine, and asparagine interact with distinct regions of the P7C3 molecule for Nampt dimer stabilization. FIG. 2B shows selected distances within a 3-5 Å range between the linker and aromatic ring regions of P7C3 with amino acids contained within the activation site (aka “tunnel”). The Glide SP based docking score calculates P7C3's relative binding free energy as −6.7 kcal/mol. The top and side views of the activation site docked with P7C3 reveal two Nampt monomers that form two distinct Nampt dimeric interfaces (FIGS. 30C-30H), wherein each interface comprises both an activation site (aka “tunnel”) and an active site (aka “catalytic site”) that are solvent accessible when neither ligands or substrates are bound.

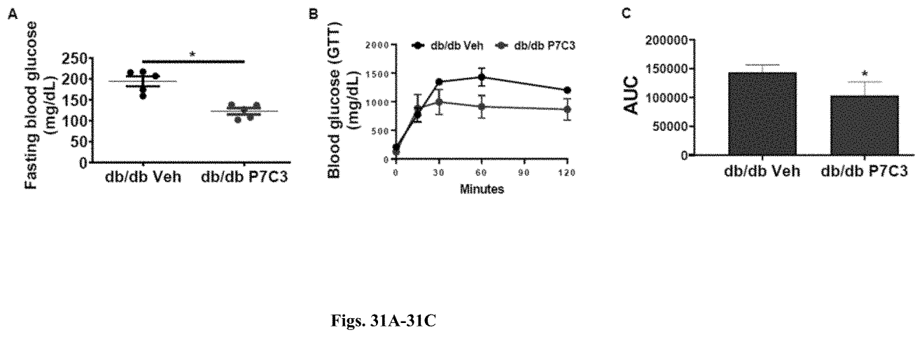

Decreased circulating blood glucose levels in P7C3 treated db/db mice. It has been recently reported that P7C3 treatment of the diabetic (db/db) mice for 4 weeks improves the circulating blood glucose levels. The present study shows a similar significant decrease in the fasting blood glucose levels of the overnight fasted db/db mice treated with P7C3 (10 mg/kg body weight/day, i.p.) for 4 weeks compared with the vehicle treated mice (FIG. 31A). The intraperitoneal glucose tolerance test of the diabetic mice treated with P7C3 for 4 weeks displayed a decrease in circulating blood glucose from 30 minutes onwards but was found to be non-significant between the treatment groups (FIG. 31B). However, the index of the total glucose shift that was calculated as the area under curve using the trapezium method displayed a significant difference between the treatment groups variances (FIG. 31C). Taken together, this confirms that P7C3 treatment enhances glucose use of the diabetic mice.

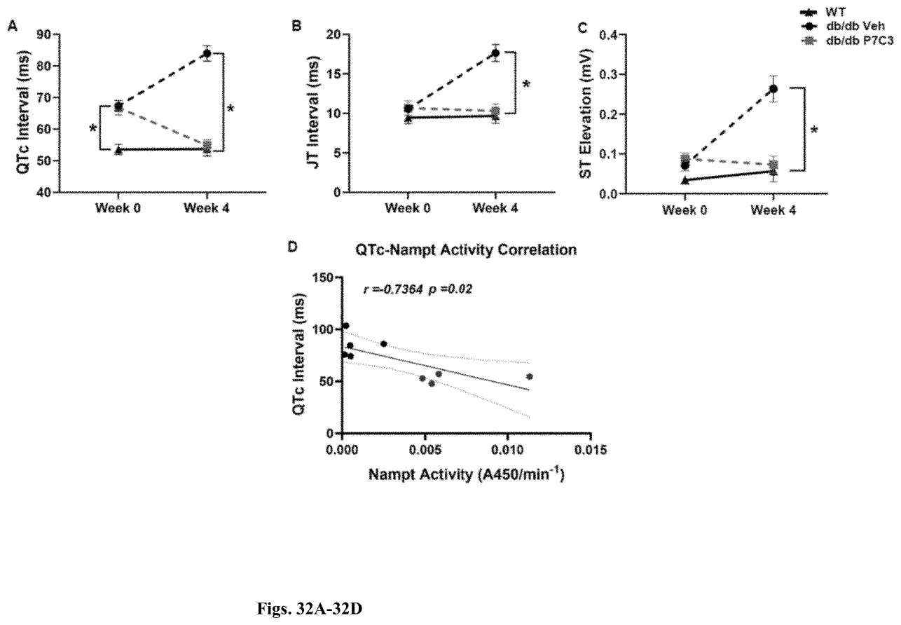

Cardiac electrical parameters are significantly reduced in P7C3 treated db/db mice. Overall in vivo cardiac function was investigated by examining key electrical parameters via surface lead ECG recordings. The QTc interval remains a critical measurement for overall cardiac health and function with diabetic patients and preclinical models demonstrating elevated QTc and JT. Here, this study demonstrates that the db/db vehicle treated mice present with significantly elevated QTc and JT intervals as well as, increased ST elevation compared with their non-diabetic wildtype controls (FIGS. 32A-32C). These intervals were significantly attenuated in db/db P7C3 (10 mg/kg body weight/day, i.p.) treated mice, with values closely aligning with their non-diabetic wildtype controls (FIGS. 32A-32C). A correlative analysis clearly identifies a significant association with increased Nampt activity and lower QTc durations (FIG. 32D). Collectively, these data demonstrate that P7C3 treated db/db mice have significantly attenuated cardiac electrical parameters compared with vehicle treated mice and the values are on par with non-diabetic control mice.

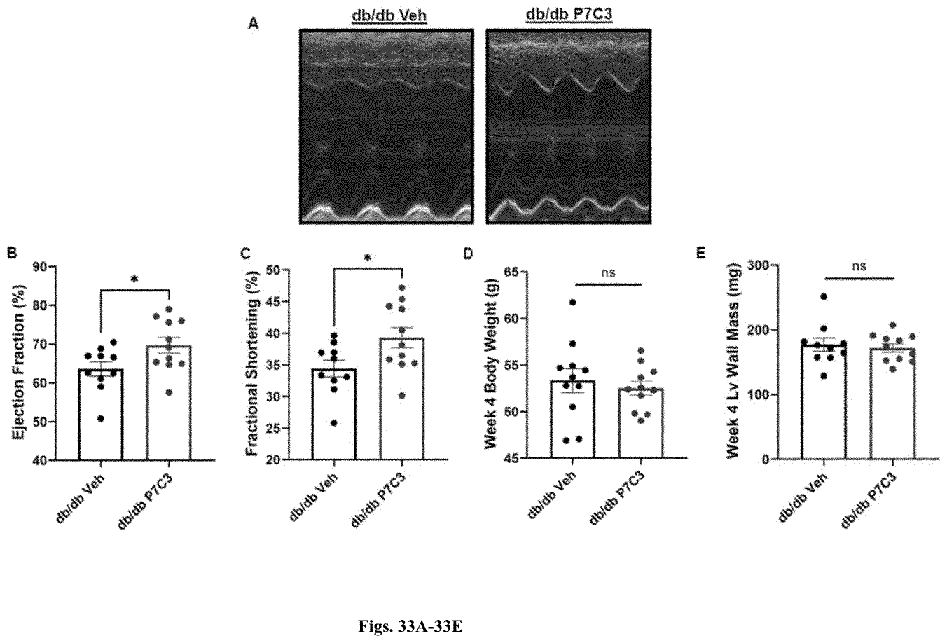

Cardiac functional parameters are significantly increased in P7C3 treated db/db mice. Transaortic echocardiography was utilized to further evaluate cardiac function and to investigate overall systolic function. The db/db P7C3 (10 mg/kg body weight/day i.p.) treated mice demonstrated improved echocardiography (FIG. 33A), and significantly increased the ejection fraction (EF %) and fractional shortening (FS %) parameters post four-weeks (FIGS. 33B and 33C). The evaluation of body weight and left ventricular heart weight demonstrated no alterations between the P7C3 and vehicle treated db/db mice (FIGS. 33D and 33E). These data show that P7C3 improved the systolic function as a result of enhanced overall cardiac function, and the echo-based evaluation did not find heart weight or size changes.

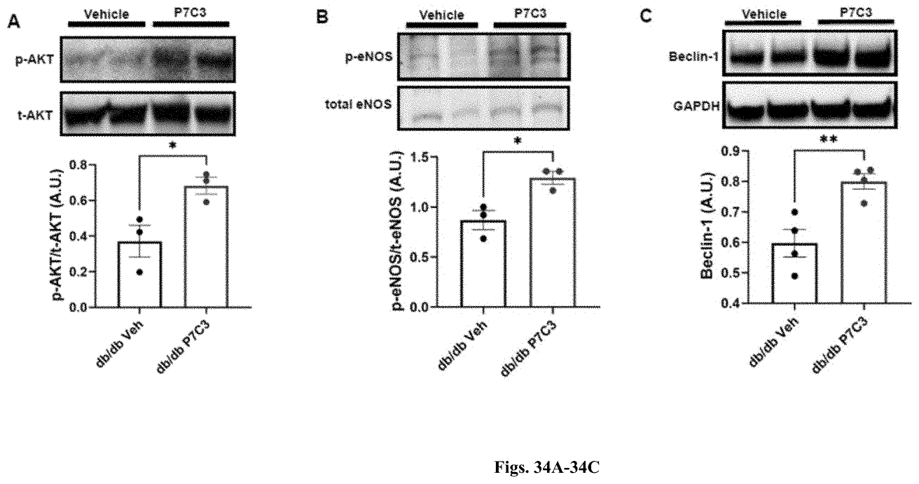

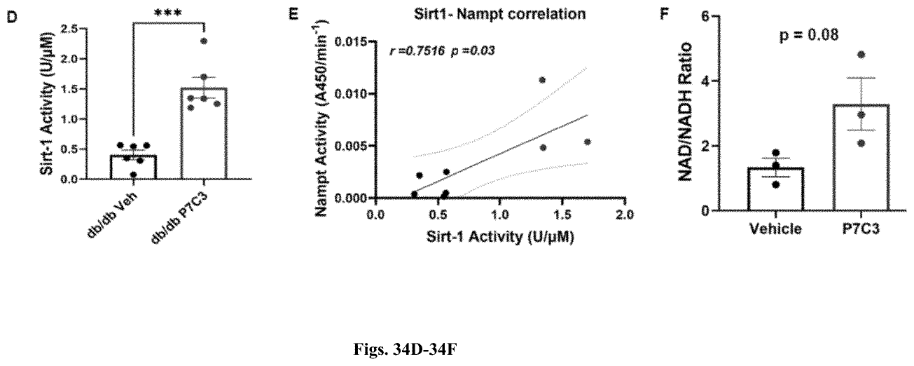

Enhanced Akt and eNOS phosphorylation, and Beclin−1 signaling with increased Sirt1 enzymatic activity of P7C3 treated db/db mice. Western blot analysis of cardiac tissue from P7C3 (10 mg/kg body weight/day i.p.) treated db/db mice demonstrated significantly increased p-AKT signaling compared with vehicle treated mice (FIG. 34A). Since eNOS is a major regulator of nitric oxide production in the heart and is key for cardioprotection, and a downstream target of p-AKT signaling, we measured eNOS^{ser1177} phosphorylation. As shown in FIG. 34B, P7C3 treatment significantly increased p-eNOS levels in cardiac tissue, indicating that the cardioprotective benefits via pAKT by P7C3 is at least in part via eNOS stimulation. Additionally, the P7C3 treated db/db mice showed a significant increase in protein expression of pro-autophagy marker, Beclin 1, when compared with that of vehicle treated mice (FIG. 34C). Examination into the NAD⁺-SiRT1 axis demonstrated a significant increase in SiRT1 activity in cardiac tissue from db/db P7C3 treated mice compared with db/db vehicle controls (FIG. 34D). A correlation analysis further highlighted a significant positive relation with Nampt and SiRT1 activity within the P7C3 treated db/db mice (FIG. 34E). Hearts from diabetic mice showed a 2.5-fold increase in cardiac the NAD⁺/NADH ratio found in P7C3 treated db/db mice compared with vehicle control (FIG. 34F).

Diabetic ischemia reperfusion injury is reduced by acute P7C3 treatment. Ex vivo model of ischemia reperfusion (I/R) injury resulted in decreased Nampt activity and elevated NADH levels, therefore the level of injury the diabetic mice hearts would develop was examined, and furthermore the beneficial effects of P7C3 treatment were tested. In a subset group of db/db mice, hearts were excised and underwent the ex vivo I/R protocol (FIG. 35A). The db/db control hearts demonstrated profound ischemic injury with an average of 60% infarct size (FIG. 35B). The db/db hearts treated with P7C3 (3 μM) just prior (15 minutes) and immediately following (2 hour reperfusion) ischemia demonstrated a significantly reduced infarct size (18%) compared with vehicle control (FIG. 35B). This elevated level of injury in db/db mice was also evident in the evaluation of cardiac effluent Troponin I levels. The db/db hearts perfused with P7C3 demonstrated a significant decrease in Troponin I levels 30 and 120 minutes post-ischemia (FIG. 35C).

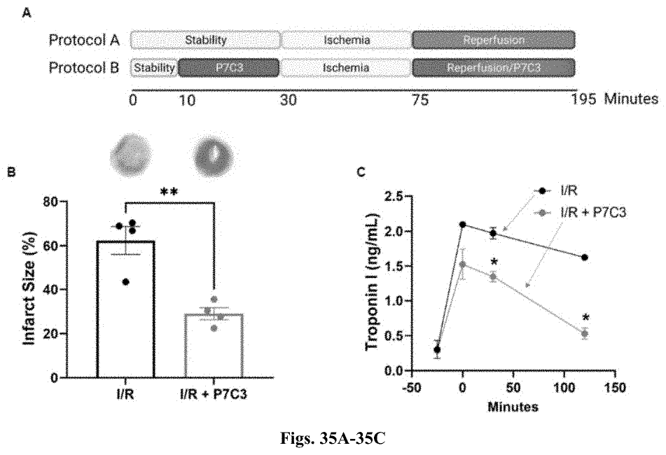

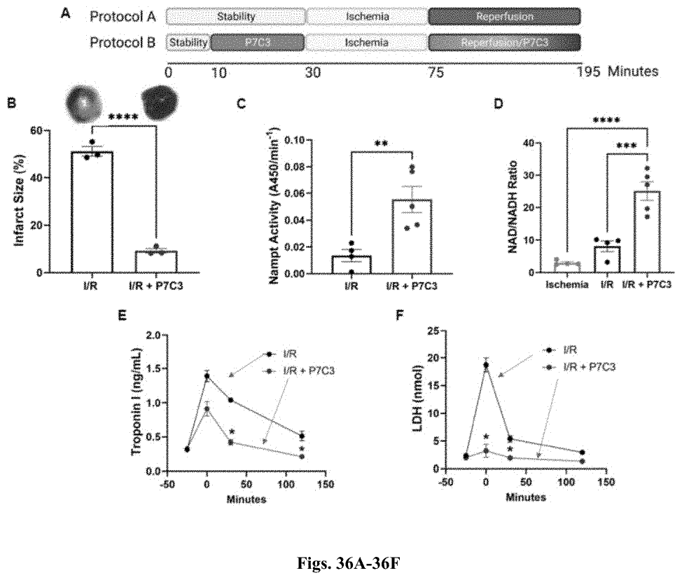

P7C3 perfusion ameliorates ischemia reperfusion injury. Ischemia reperfusion injury remains a critical component of recovery from cardiac episodes, including myocardial infarctions. Reperfusion injury is often characterized with a return of blood supply resulting in cellular cardiac death, and are often characterized with infarct zones. Reperfusion injury has been associated with a significant increase NADH levels. This study demonstrated that the pre- and post-exposure to P7C3 in an ex vivo Langendorff system provided significant protection in db/db hearts. This study, therefore, sought to establish whether P7C3 perfusion has the potential to reduce all I/R injuries by utilizing a wildtype (C57Bl/6J) mouse model (FIG. 36A). WT hearts perfused with P7C3 (3 μM) demonstrated a significant reduction in ischemic injury (8% vs. 55%) compared with WT controls (FIG. 36B). Biochemical examination demonstrated a significant 1.7-fold increase in cardiac Nampt activity within those hearts perfused with P7C3 (FIG. 36C). Corresponding NAD⁺/NADH levels demonstrated a similar significant trend with a 3-fold increase in NAD⁺/NADH levels observed in hearts perfused with P7C3 (FIG. 36D). Coronary effluents collected during ischemia further demonstrated significantly reduced levels of both Troponin I and LDH activity following I/R injury in P7C3 perfused hearts compared with control hearts (FIGS. 36E and 36F). Infarct sizes demonstrated a notable increase in overall percentage when comparing db/db vehicle and C57Bl/6J wildtype mice (Provided averages 65% vs 55%, respectively), (FIGS. 35B and 36A). Taken together this study established that the I/R injury in C57Bl/6J wildtype mice can significantly be reduced with the addition of P7C3 resulting in enhanced Nampt activity and elevated NAD⁺/NADH ratios coupled with significantly reduced cardiac infarct sizes and reduced cardiac injury markers, including Troponin I and LDH.

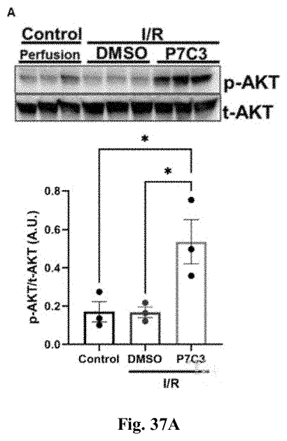

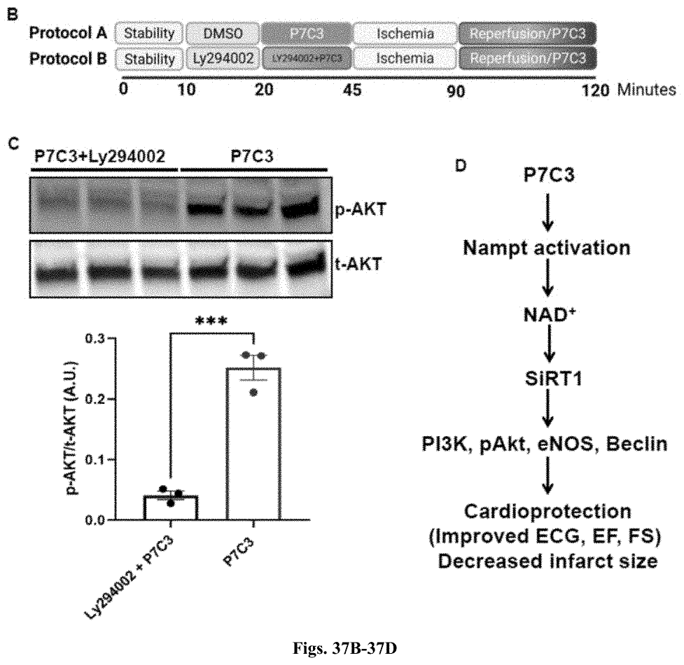

P7C3 attenuates I/R through enhancement of p-AKT. To further substantiate the reduced cardiac injury observed during I/R in C57Bl/6J wildtype hearts perfused with P7C3 (3 μM), to the study examined the overall cardiac p-AKT/t-AKT levels post I/R injury. Control hearts perfused with DMSO as vehicle control demonstrated an overall reduction in p-AKT/t-AKT levels compared with hearts undergoing simple perfusion. Hearts perfused with P7C3 demonstrated a significant increase in p-AKT/t-AKT levels when compared with vehicle control hearts (FIG. 37A). To further test if P7C3 indeed activates Akt and offers cardioprotection, the hearts were perfused with a PI3K inhibitor, LY294002 (15 μM) just prior to inducing global ischemia (FIG. 37B). This strategy allowed to block the PI3K signaling, which is upstream of Akt. As shown in FIG. 37, perfusion with PI3K inhibitor LY294002 compound attenuated the P7C3 based pAkt expression (FIG. 37C). These data show that P7C3 activated pAkt in the heart and provides cardioprotection.

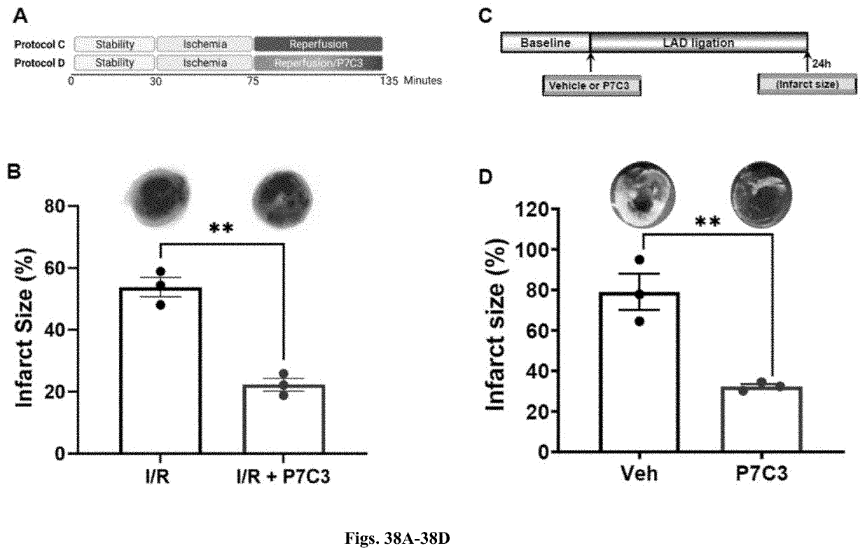

P7C3 post-ischemia exposure reduces ischemia reperfusion injury. Ischemia reperfusion injury remains a critical component of recovery from cardiac episodes, including myocardial infarctions. We demonstrated that the pre- and post-exposure to P7C3 in an ex vivo Langendorff system provided significant protection in both db/db and WT hearts. We therefore, sought to establish whether P7C3 (3 μM) perfusion post ischemia has the potential to reduce overall I/R injuries by utilizing a wildtype (C57Bl/6J) mouse model (FIG. 38A). A similar level of ischemic injury was induced in WT hearts exposed to ischemia-reperfusion protocols with a reduced reperfusion time (60 mins vs. 120 mins) (54% vs. 55%, respectively, FIGS. 38B and 36A). Wildtype hearts perfused post-ischemia with P7C3 (3 μM) demonstrated a significant reduction in overall ischemic injury (22% vs. 54%) compared with controls (FIG. 38B).

P7C3 decreases in vivo myocardial infarction. Next, we sought to determine whether P7C3 pre-treatment decreases the in vivo myocardial infarction mimicking clinical settings. The C57Bl/6J mice treated with a single bolus of P7C3 (10 mg/kg body weight/day, i.p.) 30 minutes prior to the LAD coronary artery occlusion displayed a significant reduction in the total infarct size measured 24 hours after the occlusion of the LAD coronary artery compared with control vehicle treated mice (FIGS. 38C and 38D).

This is the first report showing that P7C3 treatment leads to improved diabetic cardiac function. The cardioprotective benefits of Nampt activator; P7C3, are mediated by decreasing the cardiac NADH/NAD⁺ ratio and improving Nampt and Sirt1 activity. At the signaling level, activation of Nampt by P7C3 upregulated diabetic cardioprotective signaling with increased p-AKT, p-eNOS and autophagy signaling.

A previous study demonstrated that P7C3 administration overcomes insulin resistance and decreases blood glucose with 4-week P7C3 treatment in diabetic mice. The present study evaluated the cardiovascular benefits and signaling given that diabetes is a major risk factor for myocardial ischemia and cardiovascular related deaths.

The major cardiovascular complications of diabetes include increased risk to life threating arrhythmias and decreased cardiac function. Therefore, the propensity of this utilizing a mouse model of diabetes and rescued by Nampt activator P7C3 was evaluated. The ECG based evaluation from diabetic patients show prolonged QTc, QT interval, JT interval and T wave. Past studies in preclinical models such as db/db mice also confirm cardiac dysfunction in diabetes, including QTc prolongation, and significant systolic and diastolic dysfunction. The present study shows the cardioprotective benefits of Nampt activation by P7C3 in attenuating QTc durations and improved cardiac function as assessed by echocardiography. Echocardiographic data demonstrated that P7C3 treated db/db mice presented with significant increases both in the EF % and FS % parameters, indicating a significant improvement in LV systolic function.

Nampt is the rate-limiting enzyme in mammalian NAD⁺ salvage pathway. Nampt is key for NAD⁺ synthesis in the heart and previous research demonstrated that overexpressed Nampt increases NAD⁺ content in cardiomyocytes. Nampt is downregulated at both the protein and mRNA levels under stress conditions in the heart leading to decreased NAD⁺ level in the heart. Studies in rats showed that diabetes leads to a near 3-fold increase in intra-cardiac NADH/NAD⁺ ratio, measured as lactate/pyruvate ratio, when compared with control group. Recent murine studies using pressure overload and isoproterenol models of heart failure showed that administration of NMN, which is a product of Nampt activity, leads to normalization of NADH/NAD⁺ balance in the heart and significantly attenuates cardiac hypertrophy, LV dilatation, and improved contractile function, indicating significant cardioprotective benefits of NAD⁺ accretion. Previous studies have established pyridine nucleotides, in particular NAD⁺/NADH ratios to play a critical role in multiple regulatory pathways involved in cardiac function, including autophagy, DNA repair, antioxidant, and mitochondrial function.

Molecular docking studies show interactions between P7C3 and Nampt. These interactions indicate P7C3 to be a putative small molecule Nampt activator based on its binding and affinity to the Nampt protein. Moreover, a working hypothesis for Nampt activation by P7C3 considers the ligand's role in further stabilizing the Nampt dimeric interface, wherein upon binding of P7C3 to the Nampt “tunnel” adjacent to the catalytic site can increase the interactions between the two Nampt monomers and provides additional energetic stability to the entire Nampt dimer. Both the in vitro and in vivo evidence confirm that P7C3 activates Nampt enzyme. In addition, the present study is consistent with the beneficial effects in diabetic heart and with evidence for increased Nampt and SiRT1 activity along with increased NAD⁺ levels.

The P7C3 treated db/db mice in this study demonstrated significantly improved cardiac Nampt activity and correspondingly decreased NADH/NAD⁺ ratio concomitant to significant cardioprotection. NAD⁺ is an essential cofactor for Sirtuins and regulates their deacetylase activity, and activation of Sirtuins has been shown to protect heart from injury. Further, enhancing Sirt1 activity in db/db mice has been shown to improve cardiac function at least in part through NAD⁺-dependent correction of proteome acetylation status and improved mitochondrial function. It is thus plausible that the cardioprotective benefits of Nampt activation by P7C3 may at least in part be mediated through improving Sirtuin activity, as noted in this study, where P7C3 treatment significantly improved Sirt1 deacetylase activity.

Administration of NMN, an intermediate of NAD⁺ synthesis, to female diabetic mouse has been demonstrated to elevate hepatic NAD⁺ and AKT phosphorylation. Although animal models of diabetes differ in the basal activity of PI3K/AKT pathway in the heart, it is however consistent that insulin resistance in diabetes is associated with significant attenuation of decreased myocardial PI3K/AKT signaling. Treatment with P7C3 enhanced cardiac p-AKT levels, indicating that P7C3 alleviated cardioprotective signaling in db/db mice. Moreover, elevated p-AKT that is also suggestive of enhanced PI3K/AKT cascade in myocardium explains at least in part the QTc normalization observed in P7C3 treated db/db mice. Recent studies showed that deletion of PI3K p110α subunit prolongs action potential duration and QT interval in mice, and augmenting PI3K activity in type 1 and type 2 diabetic mice significantly reversed the action potential prolongations. It is therefore likely that the prolongation of action potential and corresponding ECG correlates such as QTc interval in db/db mice. Further, increased eNOS^{ser1177} phosphorylation that drives cardioprotective nitric oxide production in endothelial cells is downstream of AKT, and as such increased p-AKT in human endothelial cells has been shown to increase p-eNOS level, indicating the P7C3 induced cardioprotection involves PI3K/AKT/eNOS axis. Previous studies also reveal that Beclin 1 is dysregulated in db/db heart. The present study investigated the status of autophagy in P7C3 treated diabetic hearts and identified an increase in Beclin 1 expression, indicating the cardioprotective benefits.

Previous studies using anti-diabetic drugs and their utility for cardioprotection was tested for using I/R model and demonstrated protection in the heart, although the effects are comparable to P7C3, the mechanism of action for Metformin and P7C3 are different and remains under investigation. The present study shows that P7C3 activates Nampt allowing cardioprotection in diabetes, or during ischemia and ischemia-reperfusion injury. Previous studies show that NADH/NAD⁺ ratio is increased in the heart with ischemia reperfusion injury, therefore the study evaluated the role of Nampt activator P7C3 in the heart by perfusing P7C3 and demonstrate that activation of Nampt is beneficial and protects the heart by decreasing the infarct size. At biochemical level the key injury maker Troponin I was decreased and the pAkt is activated with P7C3 treatment. Overall, this study identified that the Nampt activator P7C3 is beneficial to the heart under diabetic conditions as well as during ischemia and ischemia-reperfusion injury. While pre-treatment of NAD⁺ may not always be feasible in a clinical setting, this study further investigated the potential of P7C3 solely as a post-ischemic treatment. Strikingly, post-ischemic exposure with P7C3 in wildtype hearts was able to significantly reduce overall infarct size by 2.5-fold compared with reperfusion alone. While preliminary, this experiment highlights a clinically significant avenue of investigation for those immediately experiencing episodes of myocardial ischemia and the potential to significantly reduce overall infarct tissue damage. Although, previous studies showed increased NAD⁺ is beneficial for heart via use of Nicotinamide Riboside or Nicotinamide Mono nucleotide (which supplement NAD⁺), however, in the present study we directly targeted Nampt enzyme via P7C3 small molecule. Therefore, the present study provides the cross talk between Nampt activation and its role for NAD⁺ generation and stimulation of SiRT1, along with pAkt and other beneficial markers allowing for cardioprotection (FIG. 37D). These studies confirm the specificity of Nampt activator P7C3 for the first time in a pharmacologically relevant manner.

Conclusion. The results herein demonstrate that Nampt activation with P7C3 offers a potential avenue for cardioprotection. Mechanistically, this study identified that Nampt regulation and its activation by P7C3 in the diabetic mice is key for increased intracardiac Nampt and Sirt1 activity, decreased NADH/NAD⁺, improved p-AKT, p-eNOS and Beclin 1 expression and overall improved cardiac function (FIG. 37D). The addition of P7C3 may also provide a critical prevention and treatment avenues for cardiac ischemia/reperfusion injury. Acute perfusion prior to and during reperfusion resulted in a significant decrease in total infarct size in both diabetic and non-diabetic cardiac models. The P7C3 pre-treatment also significantly decreased the myocardial infarct size in the wildtype mice. Further investigation on developing an optimal timing window can provide greater mechanistic insight and provide a more clinically relevant and meaningful conclusion.

What is claimed is:

1. A method of treating or preventing a muscular disease in a subject in need thereof, comprising administering to the subject a therapeutically effective amount of a nicotinamide adenine dinucleotide (NAD) activator or a pharmaceutically acceptable salt thereof.

2. The method of claim 1, wherein the muscular disease is a muscle strain injury or muscle atrophy.

3. The method of claim 1, wherein the muscular disease is a diabetes-associated skeletal muscular disorder.

4. The method of claim 1, wherein the NAD activator improves muscle contractility and function.

5. The method of claim 1, wherein the NAD activator reduces a level of an inflammatory cytokine.

6. The method of claim 1, wherein the NAD activator improves the regeneration of intramuscular nerves.

7. The method of claim 1, wherein the NAD activator is selected from the group consisting of 3,6-Dibromo-a-[(phenylamino)methyl]-9H-carbazole-9-ethanol (P7C3), P7C3 A20, P7C3 S243, and a pharmaceutically acceptable salt thereof.

8. The method of claim 1, wherein the NAD activator is encapsulated within or associated with a nanoparticle.

9. The method of claim 7, wherein the nanoparticle comprises poly(lactic-co-glycolic acid) (PLGA) or polyethylene oxide (PEG).

10. The method of claim 7, wherein the nanoparticle further comprises a muscle cell-targeting agent.

11. The method of claim 10, wherein the muscle cell-targeting agent is an A01B aptamer.

12. A nanoparticle comprising
a polymer,
a nicotinamide adenine dinucleotide (NAD) activator or a pharmaceutically acceptable salt thereof, and
a muscle cell-targeting agent.

13. The nanoparticle of claim 12, wherein the polymer comprises poly(lactic-co-glycolic acid) (PLGA) or polyethylene oxide (PEG).

14. The nanoparticle of claim 12, wherein the NAD activator is encapsulated within or associated with the nanoparticle.

15. The nanoparticle of claim 12, wherein the NAD activator is selected from the group consisting of 3,6-Dibromo-a-[(phenylamino)methyl]-9H-carbazole-9-ethanol (P7C3), P7C3 A20, P7C3 S243, and a pharmaceutically acceptable salt thereof.

16. The nanoparticle of claim 12, wherein the muscle cell-targeting agent is an A01B aptamer.

17. A pharmaceutical composition comprising the nanoparticle of claim 12 and a pharmaceutically acceptable carrier.

18. A method of treating or preventing a muscular disease in a subject in need thereof, comprising administering to the subject a therapeutically effective amount of the nanoparticle of claim 12.

19. A method of improving muscular function in a subject, comprising administering to the subject a therapeutically effective amount of the nanoparticle of claim 12.

20. The method of claim 19, wherein the subject has a muscular disease.
